(19) United States
(12) Patent Application Publication
(10) Pub. No.: US 2026/0159905 A1
(43) Pub. Date: Jun. 11, 2026

(54) METHODS FOR SEX-SORTING INSECTS

(71) Applicant: The Regents of the University of California, Oakland, CA (US)

(72) Inventors: Omar Akbari, La Jolla, CA (US); Shih-che Weng, La Jolla, CA (US)

(21) Appl. No.: 19/124,061

(22) PCT Filed: Oct. 23, 2023
(86) PCT No.: PCT/US2023/077551

§ 371 Date: Apr. 24, 2025

Related U.S. Application Data
(60) Provisional application No. 63/418,783, filed on Oct. 24, 2022.

(51) Int. Cl. C12Q 1/6897; C12Q 1/6879; C12Q 1/6888

(52) U.S. Cl. CPC C12Q 1/6897; C12Q 1/6879; C12Q 1/6888; C12Q 2600/124; C12Q 2600/158

(57) ABSTRACT

Provided herein are methods of sex-sorting a plurality of insects based on sex-specific gene expression, the method comprising (a) generating an exogenous nucleic acid molecule; (b) delivering the exogenous nucleic acid molecule into an insect from the plurality of insects, wherein the exogenous nucleic acid molecule comprises a promoter region, a sex-specific splicing module, a reporter gene, and a transcription terminator; (c) detecting sex-specific gene expression of the reporter gene; and (d) sorting the insect from the plurality of insects based on the detecting of the sex-specific gene expression in step (c), thereby sex-sorting the insect based on the sex-specific gene expression.

### CROSS-REFERENCE TO RELATED APPLICATIONS

This application claims priority to U.S. Provisional Patent Application No. 63/418,783, filed on Oct. 24, 2022. The disclosure of the prior application is considered part of the disclosure of this application and is incorporated herein by reference in its entirety.

### SEQUENCE LISTING

This application contains a Sequence Listing that has been submitted electronically as an XML file named “15670-0368WO1.XML.” The XML file, created on Oct. 23, 2023, is 77.956 bytes in size. The material in the XML file is hereby incorporated by reference in its entirety.

### BACKGROUND

Gender separation is a rate-limiting step in insect release method for biological control. For many insect species (e.g., Ae. aegypti, Anopheles spp., Culex spp., D. melanogaster, An. gambie, Cx. quinquefasciatus) separating males from females early in development is considered to be impossible. A system that allows for high-throughput sex-sorting is in need, since this would enable the application of sterile insects to be expanded to many species, thereby limiting the need for harmful pesticides.

### SUMMARY

Provided herein are methods of sex-sorting a plurality of insects based on sex-specific gene expression, the method comprising (a) generating an exogenous nucleic acid molecule; (b) delivering the exogenous nucleic acid molecule into an insect from the plurality of insects, wherein the exogenous nucleic acid molecule comprises a promoter region, a sex-specific splicing module, a reporter gene, and a transcription terminator; (c) detecting sex-specific gene expression of the reporter gene; and (d) sorting the insect from the plurality of insects based on the detecting of the sex-specific gene expression in step (c), thereby sex-sorting the insect based on the sex-specific gene expression.

In some embodiments, the exogenous nucleic acid molecule further comprises a piggyBac inverted terminal repeat located at each end of an effector region. In some embodiments, the promoter region comprises a Hr5IE1 promoter or an OpIE-1 promoter.

In some embodiments, step (b) comprises integrating the exogenous nucleic acid molecule into the genome of the insect. In some embodiments, step (d) comprises sorting the insect in a larval stage.

In some embodiments, the sex-specific splicing module comprises an endogenous sex-specific exonic sequence and a truncated sex-specific intronic sequence. In some embodiments, the sex-specific splicing module is a male-specific splicing module. In some embodiments, the sex-specific splicing module is a female-specific splicing module. In some embodiments, the insect is Aedes aegypti, Drosophila melanogaster, Drosophila suzukii, Ceratitis capitata, or Anastrepha ludens. In some embodiments, the sex-specific splicing module is derived from Ae. aegypti doublesex (AaeDsx). C. capitata transformer (traF), D. melanogaster traF, D. suzukii traF. In some embodiments, the sex-specific splicing module is derived from AaeDsx, and wherein the male-specific splicing module comprises exon 4, exon 6, or any combinations thereof. In some embodiments, the sex-specific splicing module is derived from AaeDsx, and wherein the female-specific splicing module comprises exon 4, exon 5b, exon 6, or any combinations thereof. In some embodiments, exon 5b is an engineered exon 5b, wherein one or more stop codons are excluded from the exon 5b.

In some embodiments, the reporter gene comprises a DsRed gene, an EGFP gene, or any combinations thereof. In some embodiments, the insect is sorted as male based on the expression of the EGFP gene. In some embodiments, the insect is sorted as female based on the expression of the DsRed gene. In some embodiments, the transcription terminator comprises a SV40 poly(A) signal.

Also provided herein are methods of identifying the sex of an insect based on sex-specific gene expression, the method comprising (a) generating an exogenous nucleic acid; (b) delivering the exogenous nucleic acid molecule into an insect, wherein the exogenous nucleic acid molecule comprises a promoter region, a sex-specific splicing module, a reporter gene, and a transcription terminator; and (c) identifying sex-specific gene expression of the reporter gene, thereby identifying the sex of the insect based on the sex-specific gene expression.

In some embodiments, the exogenous nucleic acid molecule further comprises a piggyBac inverted terminal repeat located at each end of an effector region. In some embodiments, the promoter region comprises a Hr5IE1 promoter or an OpIE-1 promoter.

In some embodiments, step (b) comprises integrating the exogenous nucleic acid molecule into the genome of the insect. In some embodiments, step (d) comprises sorting the insect in a larval stage.

In some embodiments, the sex-specific splicing module comprises an endogenous sex-specific exonic sequence and a truncated sex-specific intronic sequence. In some embodiments, the sex-specific splicing module is a male-specific splicing module. In some embodiments, the sex-specific splicing module is a female-specific splicing module. In some embodiments, the insect is Aedes aegypti, Drosophila melanogaster, Drosophila suzukii, Ceratitis capitata, or Anastrepha ludens. In some embodiments, the sex-specific splicing module is derived from Ae. aegypti doublesex (AaeDsx). C. capitata transformer (traF), D. melanogaster traF, D. suzukii traF. In some embodiments, the sex-specific splicing module is derived from AaeDsx, and wherein the male-specific splicing module comprises exon 4, exon 6, or any combinations thereof. In some embodiments, the sex-specific splicing module is derived from AaeDsx. and wherein the female-specific splicing module comprises exon 4, exon 5b, exon 6, or any combinations thereof. In some embodiments, exon 5b is an engineered exon 5b, wherein one or more stop codons are excluded from the exon 5b.

In some embodiments, the reporter gene comprises a DsRed gene, an EGFP gene, or any combinations thereof. In some embodiments, the insect is sorted as male based on the expression of the EGFP gene. In some embodiments, the insect is sorted as female based on the expression of the DsRed gene. In some embodiments, the transcription terminator comprises a SV40 poly(A) signal.

### BRIEF DESCRIPTION OF DRAWINGS

FIG. 1A shows a construct of SEPARATOR in Ae. aegypti that used sex-specific splicing module of AaDsx. The sex-specific splicing module of AaDsx was used to construct SEPARATOR in Ae. aegypti. The expression of SEPARATOR was driven by the constitutive baculovirus promoter, Hr5Ie1. The male-specific splicing product was in-frame with the EGFP coding sequence, while inclusion of stop codons in exon 5b prevented in-frame expression of the DsRed in females. The SV40 pA served as the polyadenylation signal.

FIG. 1B shows an exemplary schematic for generating homozygotes by crossing GFP-positive males with GFP-negative females. GFP-positive larvae were sorted and the sex of each was determined at the pupal stage. GFP-positive males were then crossed with GFP-negative females in order to produce homozygotes.

FIG. 1C shows 100% of the GFP-positive mosquito larvae are male exclusively in 15 generations using the strategy in FIG. 1B. Mosquitoes were sorted by their GFP signal at the larval stage and used a microscope to examine the sex ratio based on the morphological differences in genital lobe shape at pupal stage, which are specific to each sex.

FIG. 1D shows photos of the embryo, larva, pupa, and adult stages of wild-type (Liverpool) and SEPARATOR mosquitoes that were collected and photographed using a fluorescent stereomicroscope (Leica M165FC). Eggs, aged between 24-48 hours after being laid, were subjected to a 15-30 minute treatment with 30% NaOCl solution (containing approximately 3.6% active chlorine at the final concentration) to remove the chorion and enable visualization of the embryo. Eggs were hatched in deionized water within a vacuum chamber, and the resulting hatched larvae were then collected as L1 larvae.

FIG. 1E shows the developmental stages of mosquitoes that were collected and photographed using a fluorescent stereomicroscope (Leica M165FC). The images consist of two panels: the upper panel displays the bright-field images, while the lower panel showcases the GFP/mCH channel images.

FIG. 2A shows an exemplary schematic diagram of large-scale sex-sorting processes using a Complex Object Parametric Analyzer and Sorter (COPAS®). The eggs of transgenic mosquitoes, which have been engineered with the SEPARATOR system, were incubated in a vacuum chamber filled with deionized water to hatch them. 24 hours post-egg hatching, the larvae expressing GFP were screened using the COPAS® instrument. The GFP-positive larvae were then carefully sorted, and raised in a controlled environment until they reached adulthood. Once the adults have reached maturity, their sexes were verified through various methods.

FIG. 2B shows an exemplary sorting result that is determined by the larvae's opacity and size, followed by the intensity of their GFP expression. The eggs of transgenic mosquitoes that were genetically engineered to carry the SEPARATOR system were incubated in a vacuum chamber using deionized water. After 24 hours of incubation, the hatched larvae were passed through a COPAS® instrument. To ensure accurate sorting, the larvae were selected based on both their opacity and size, and then sorted by the intensity of their GFP expression.

FIGS. 3A-3C show comparison of the transcriptome across larvae and pupae stages of mosquito. SEPARATOR mosquitoes were utilized to individually segregate male and female mosquitoes at

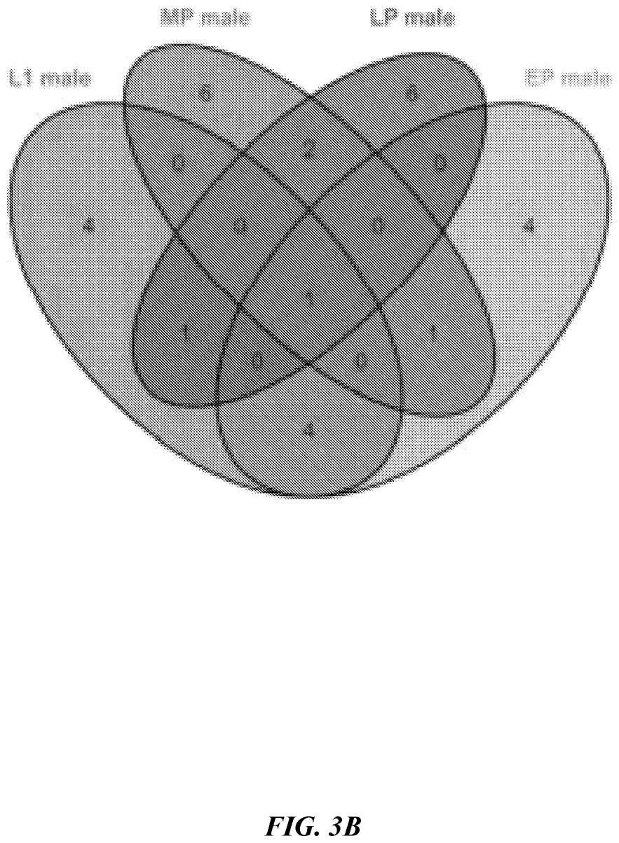

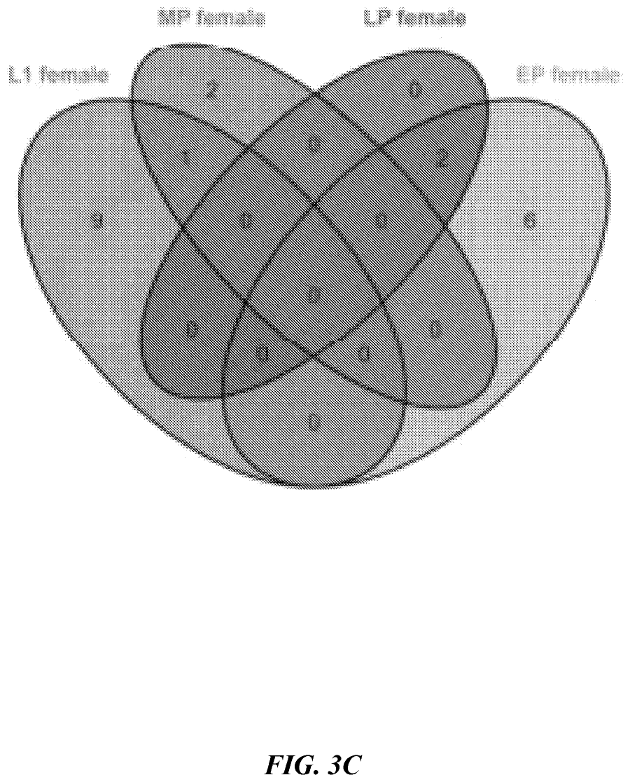

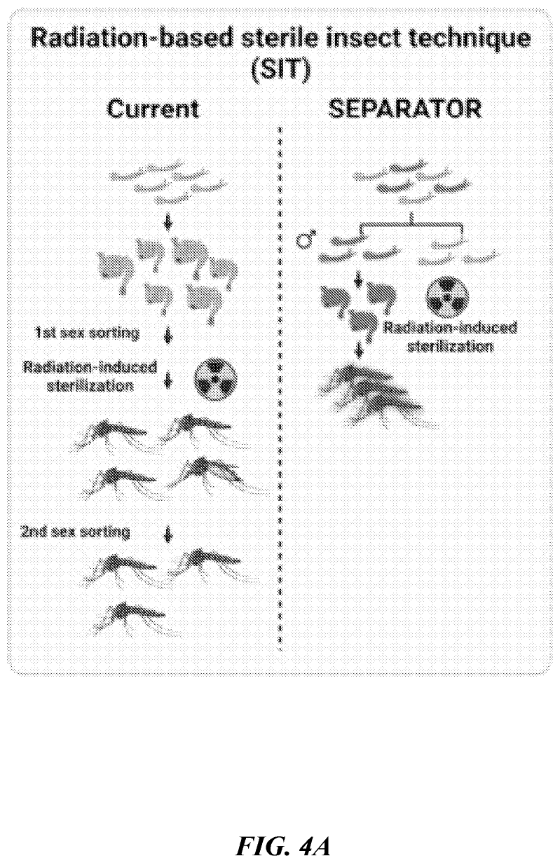

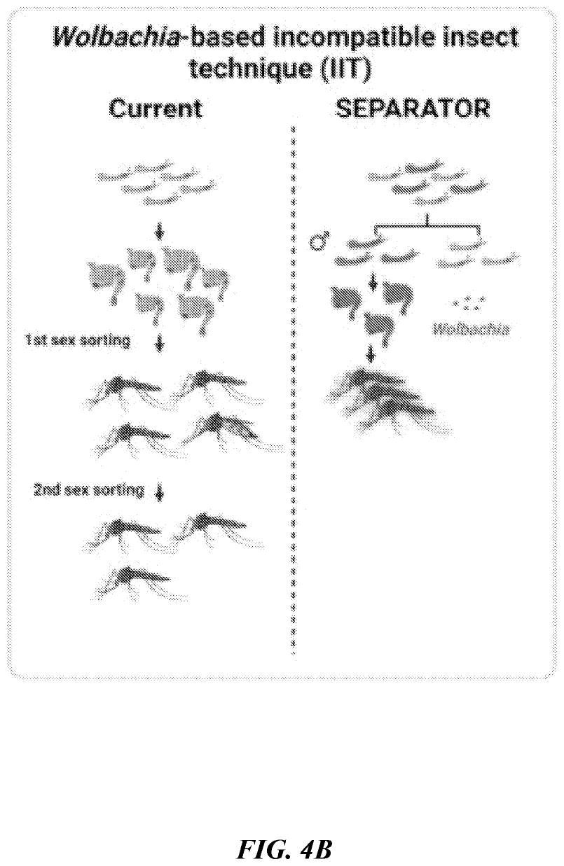

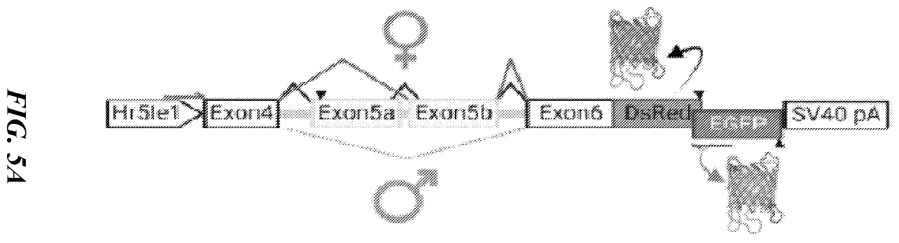

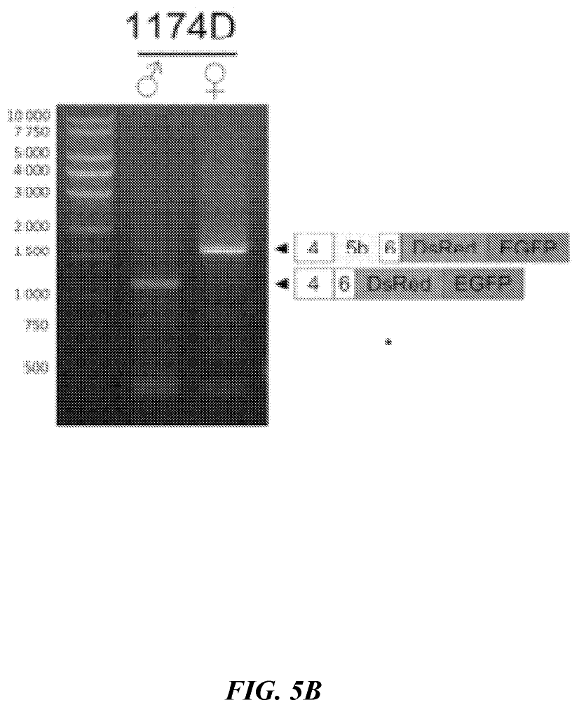

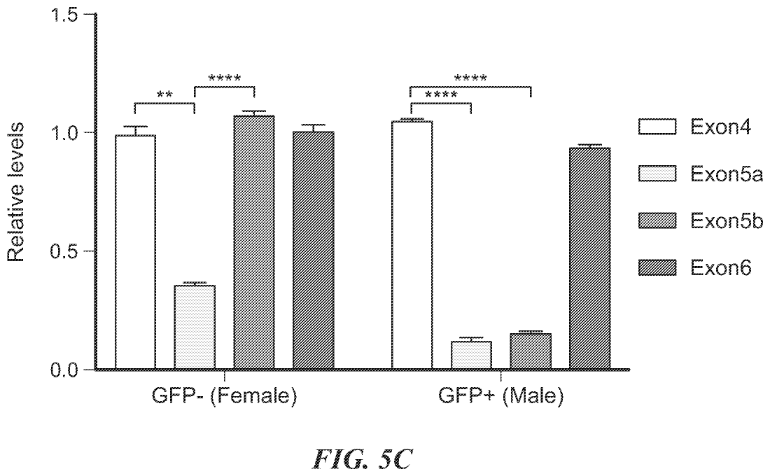

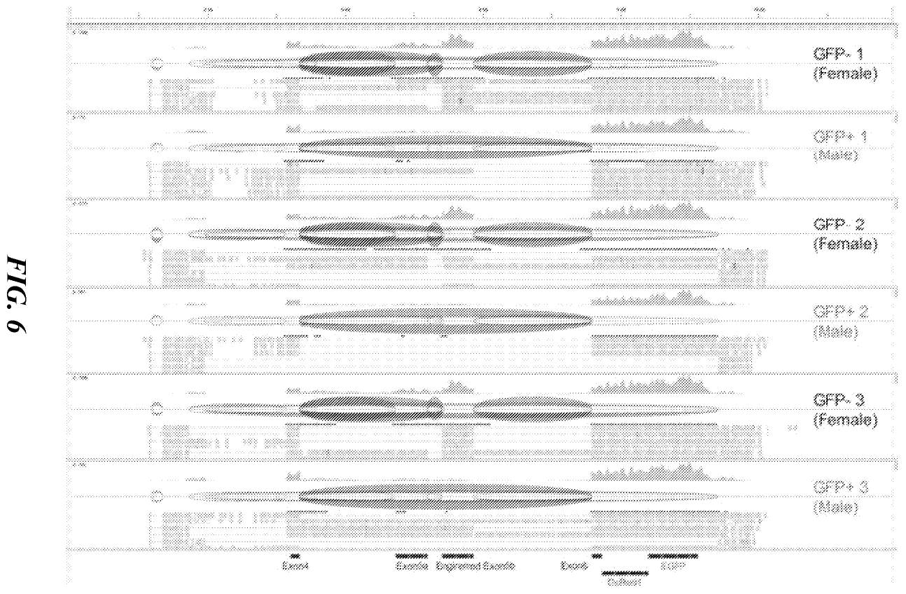

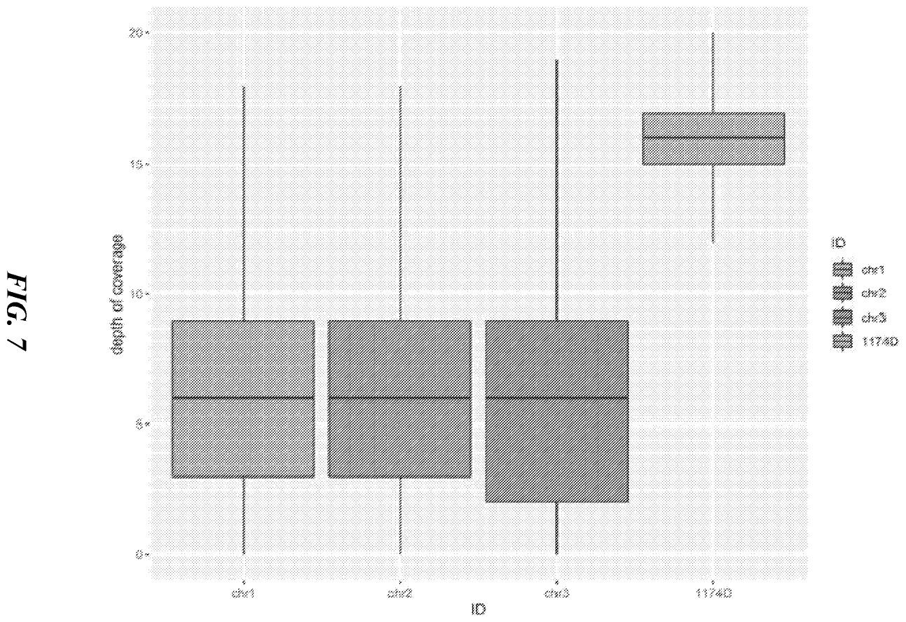

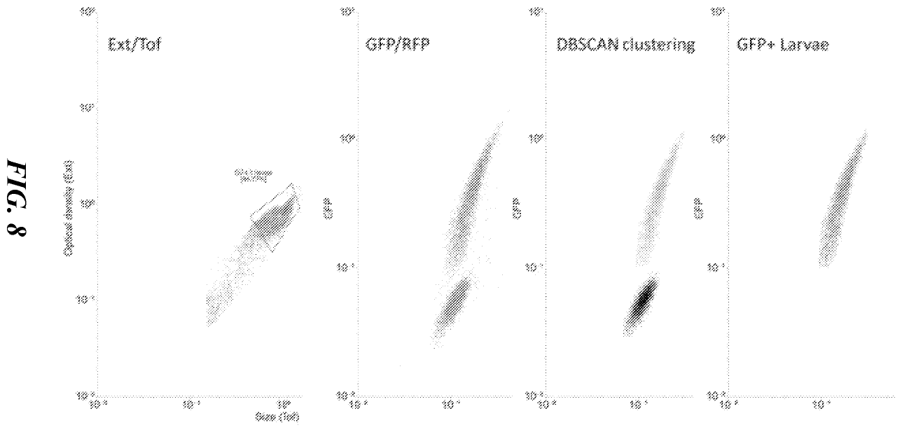

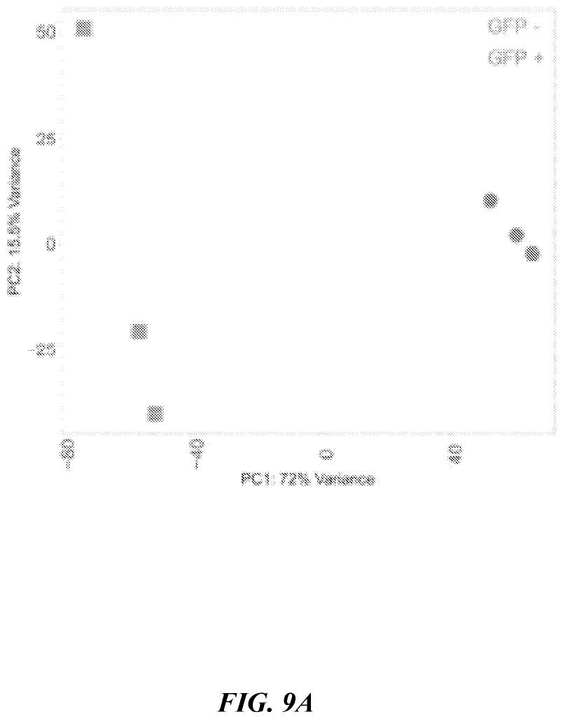

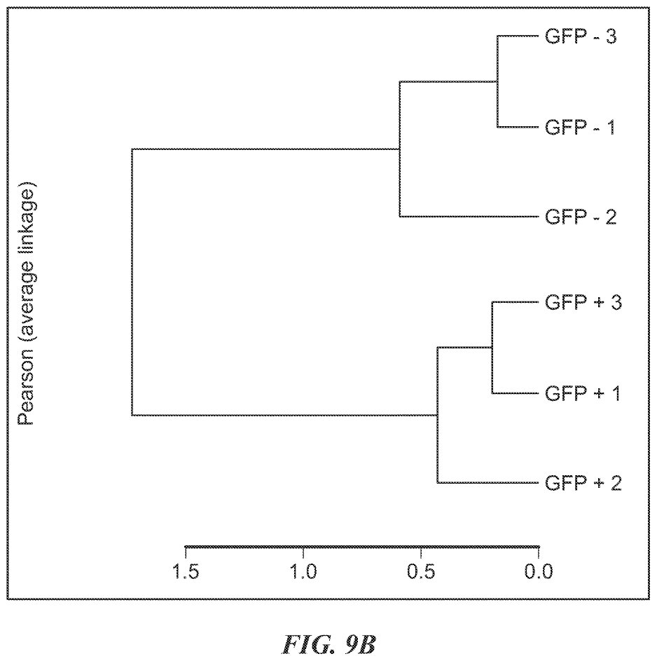

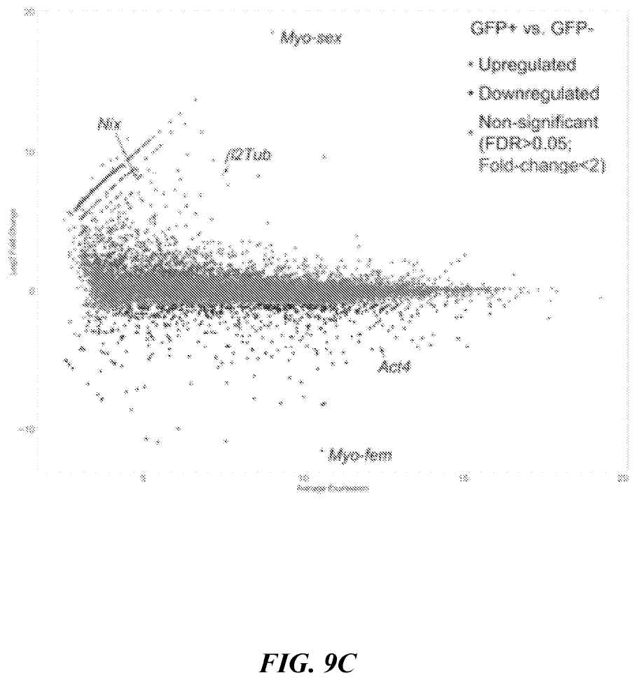

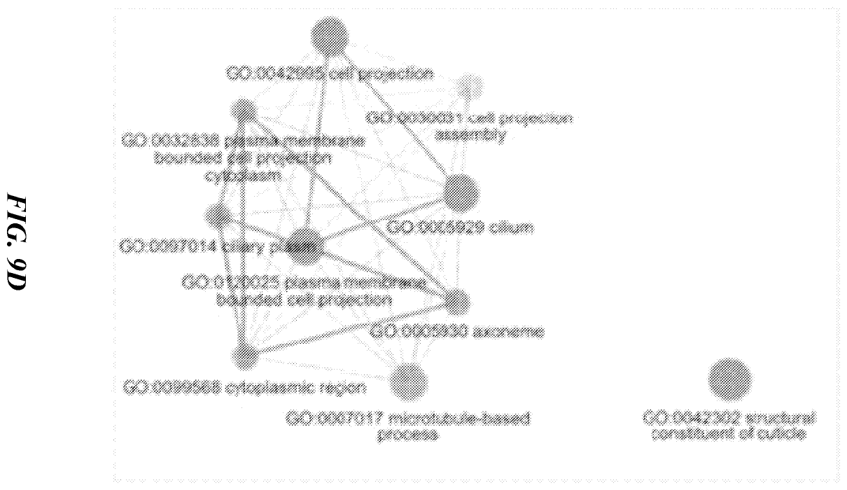

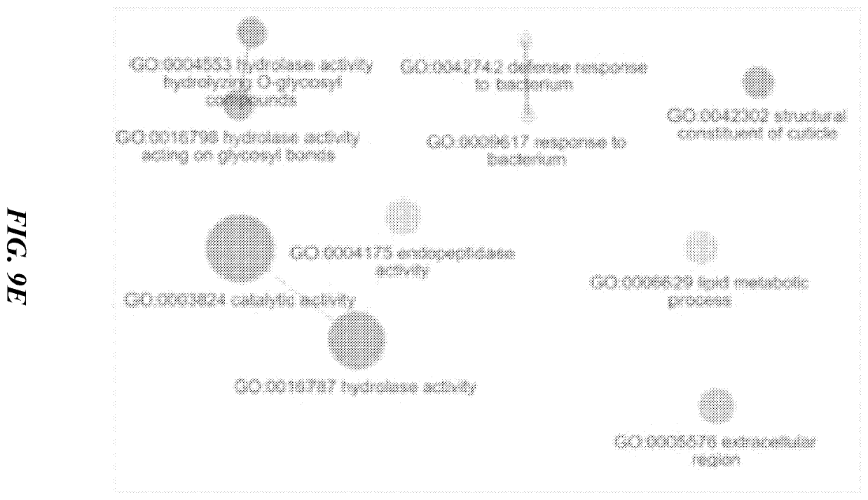

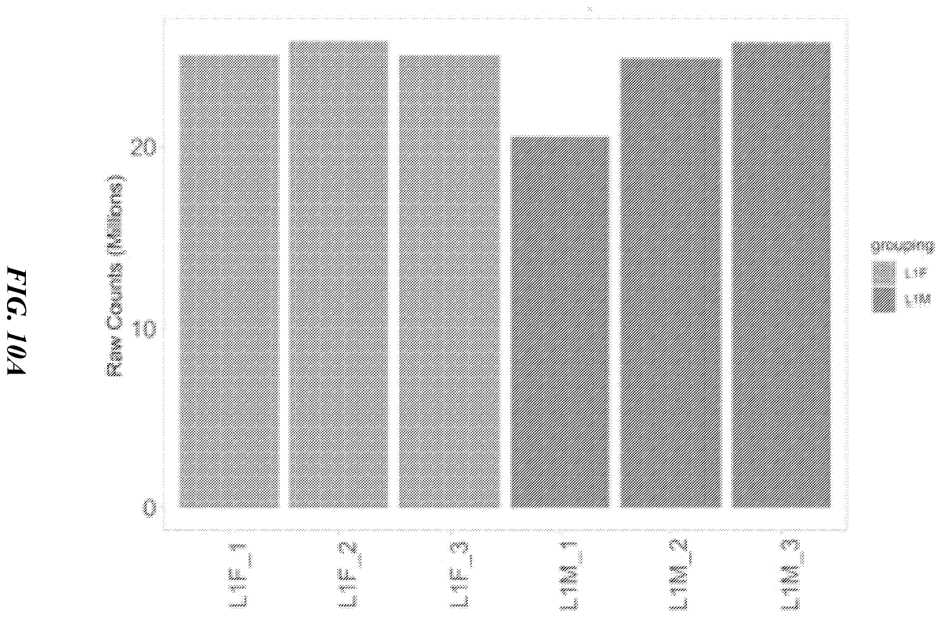

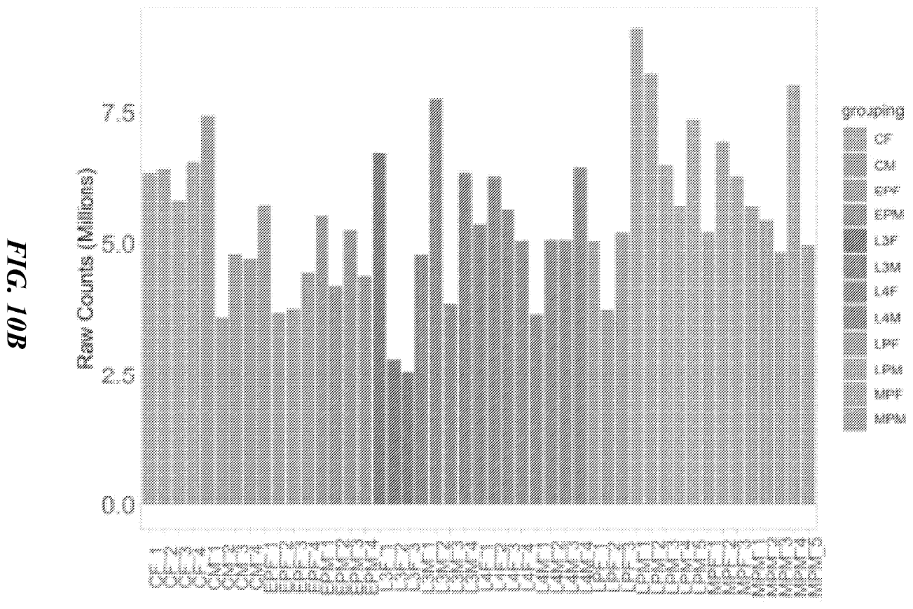

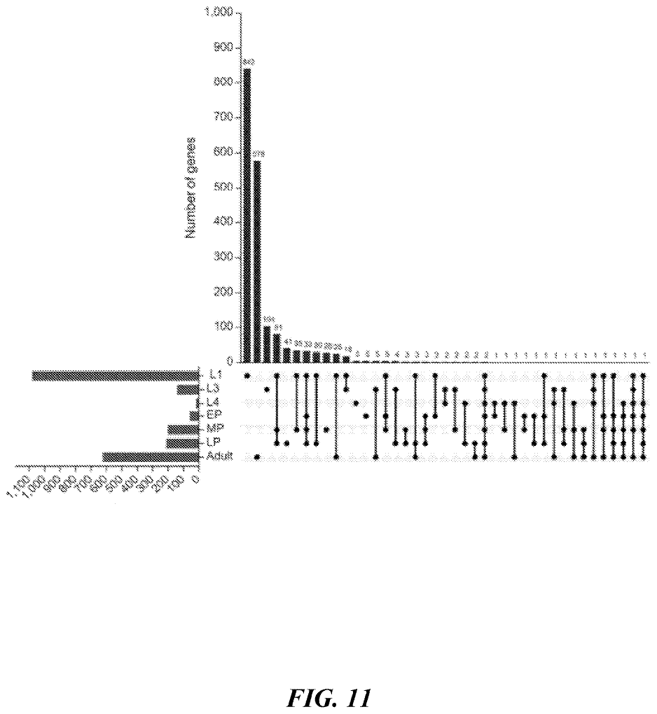

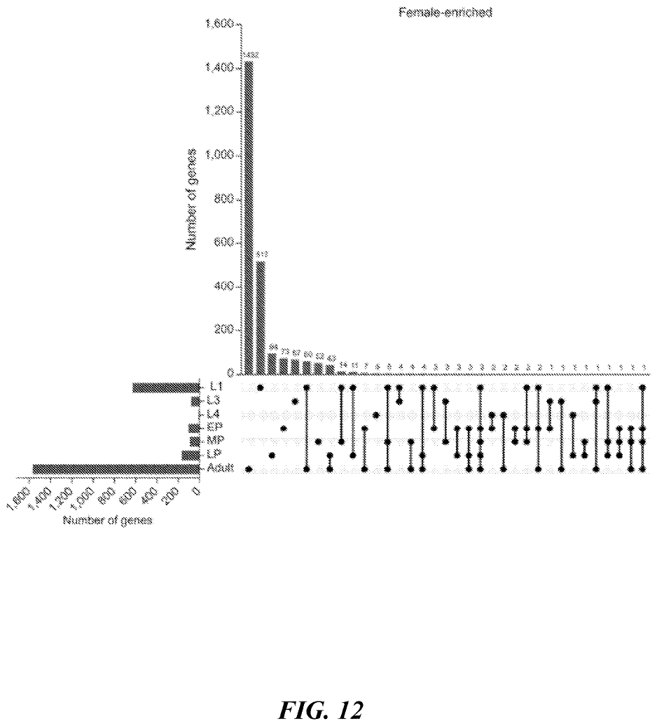

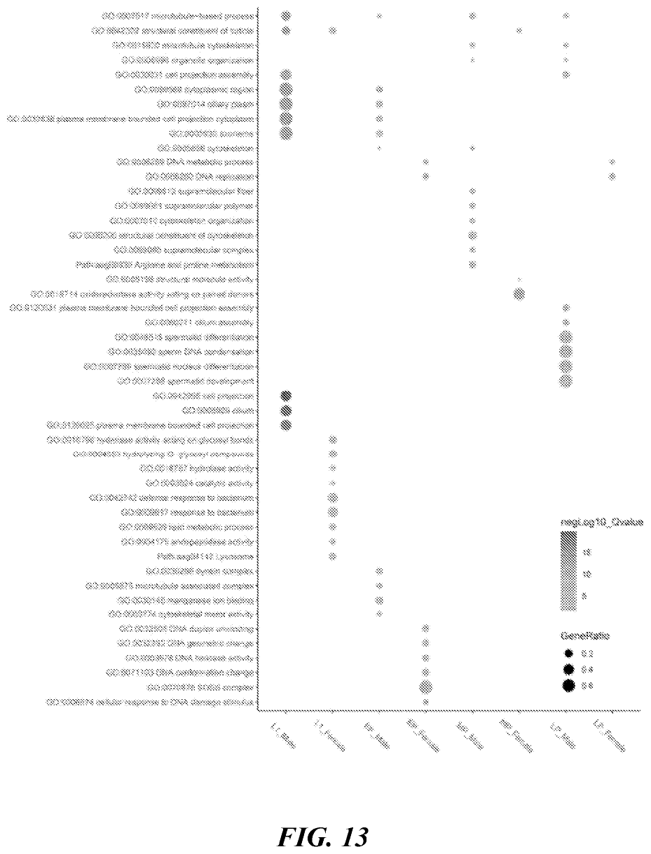

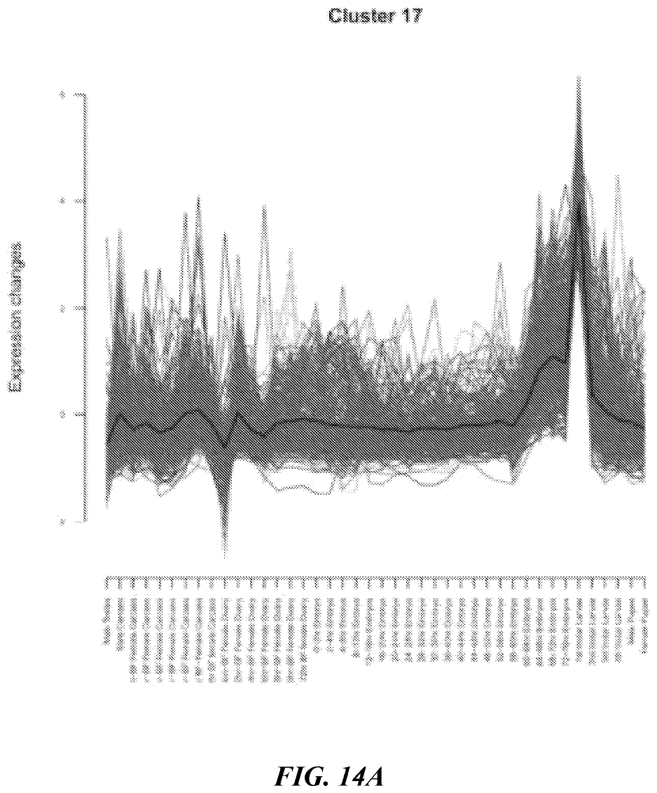

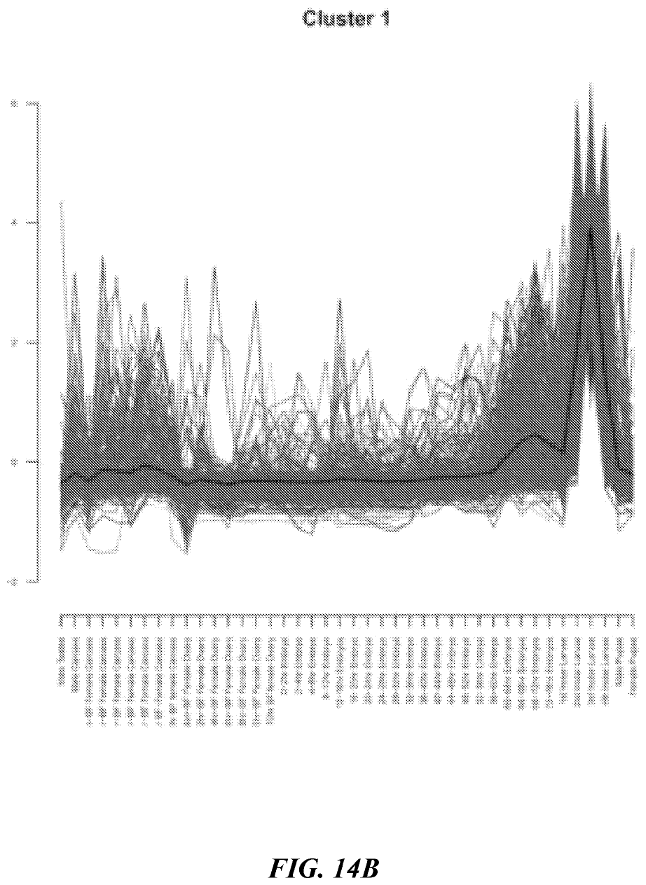

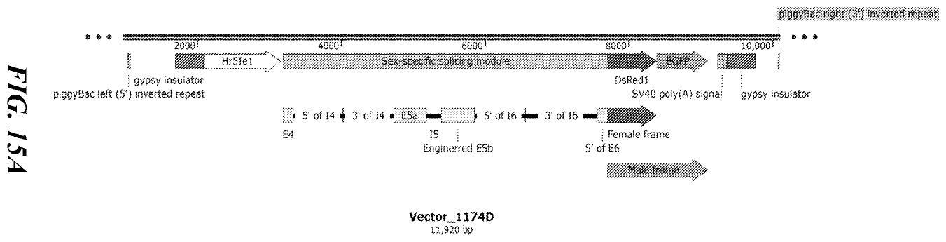

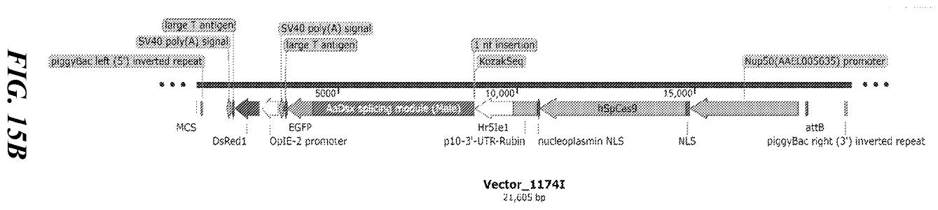

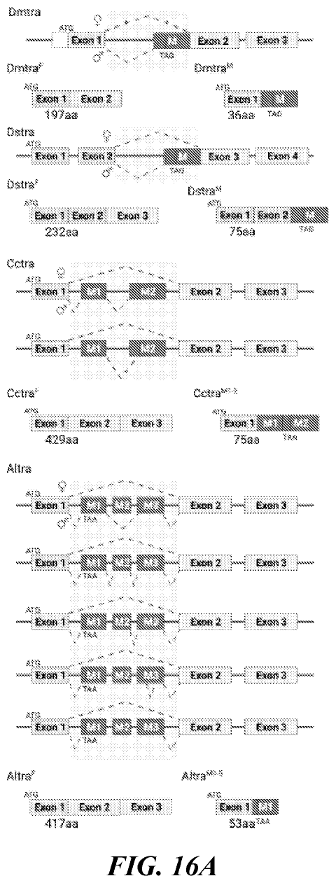

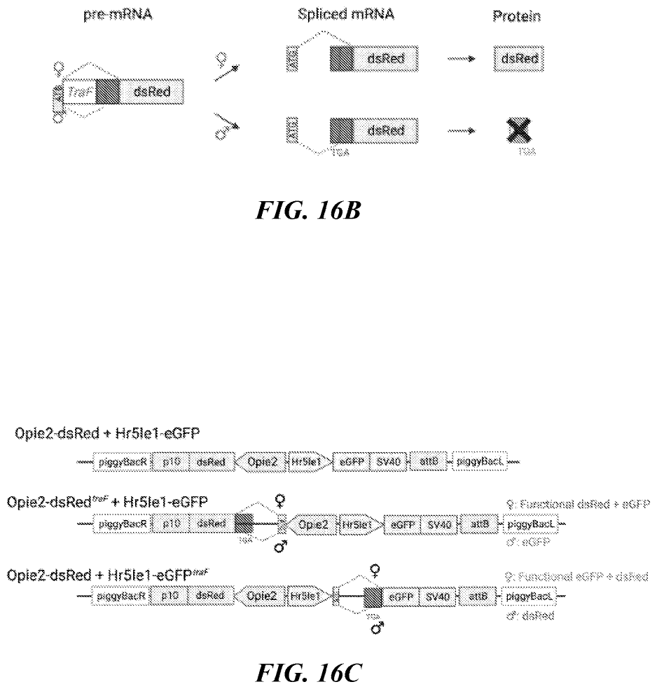

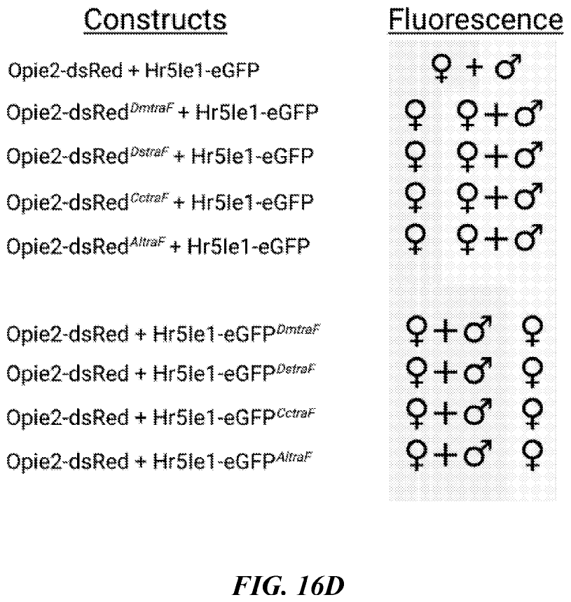

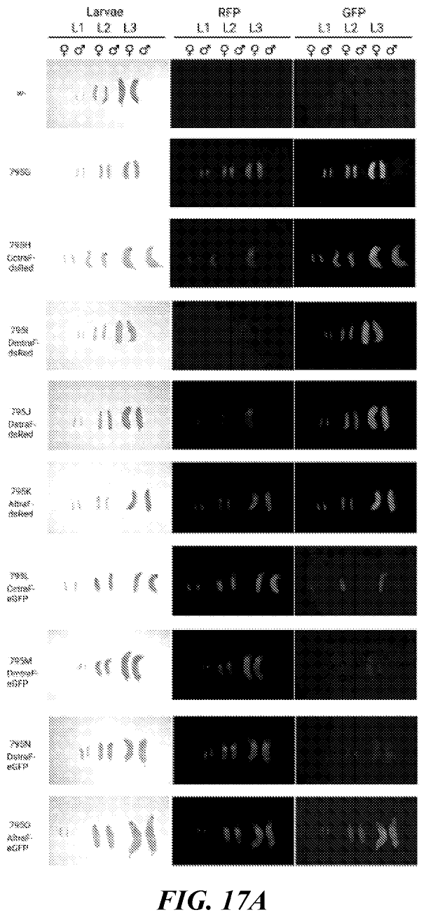

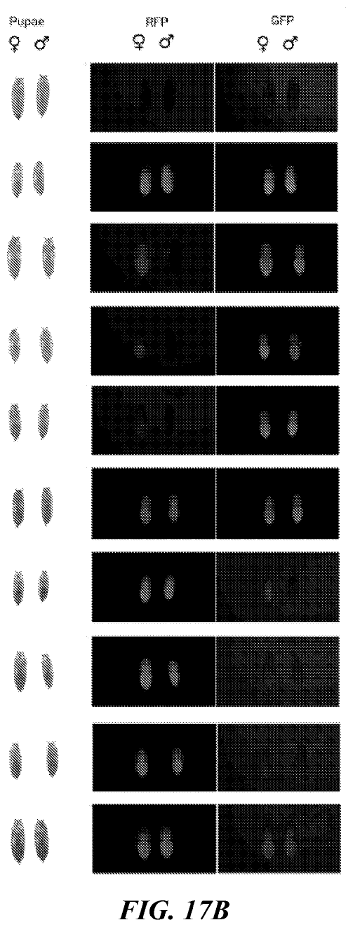

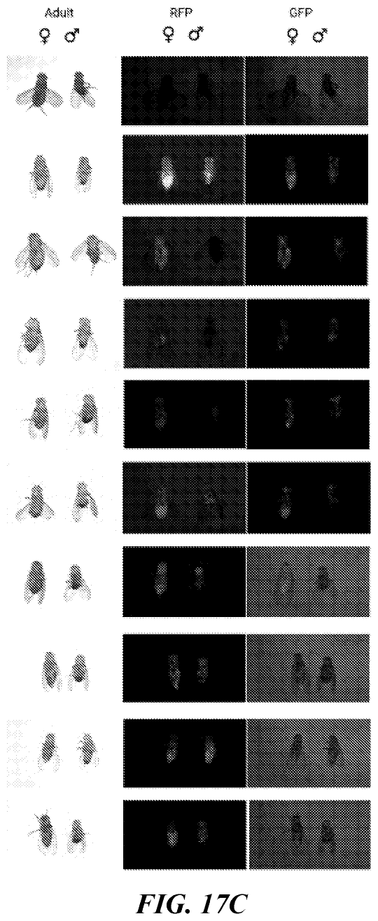

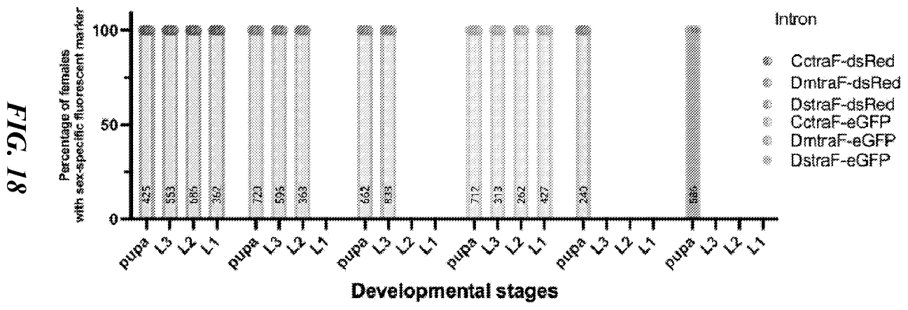

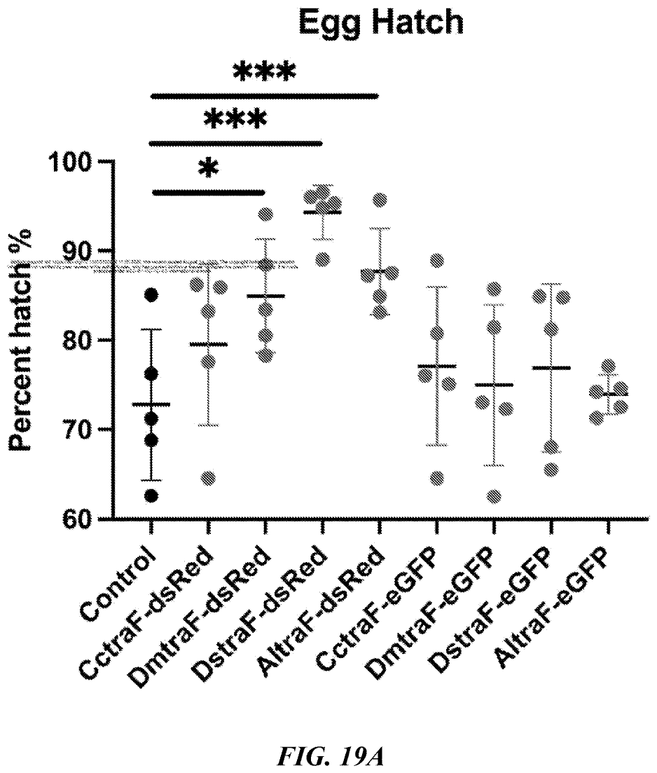

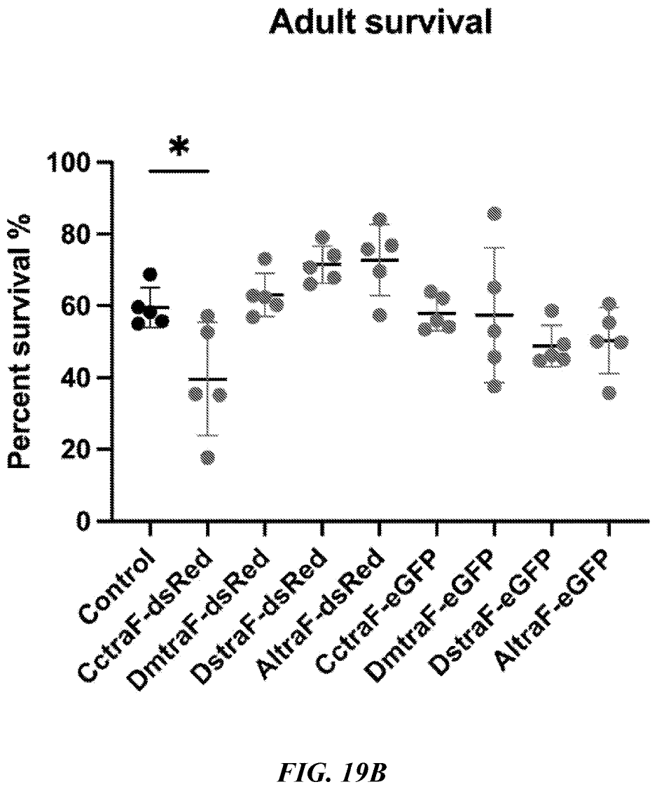

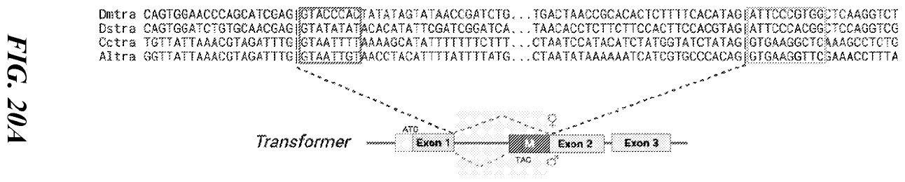

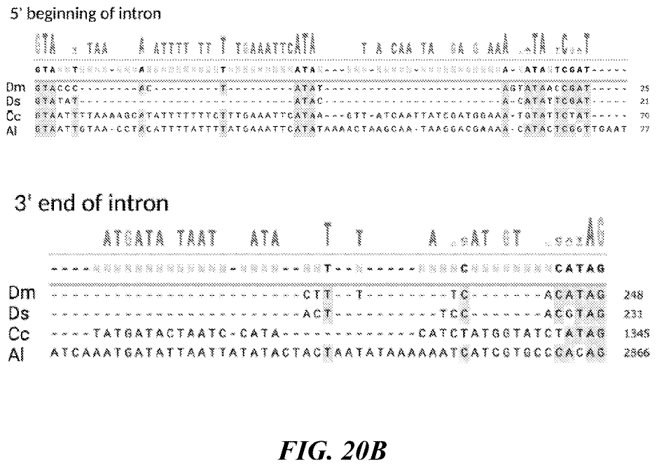

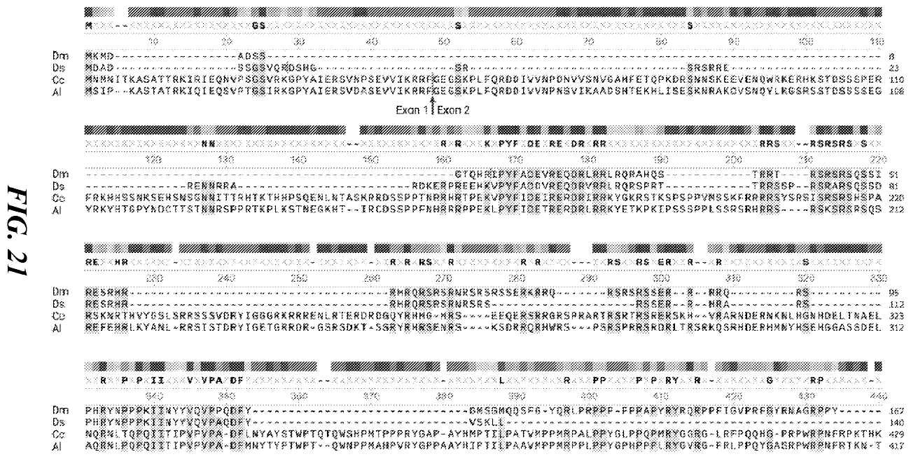

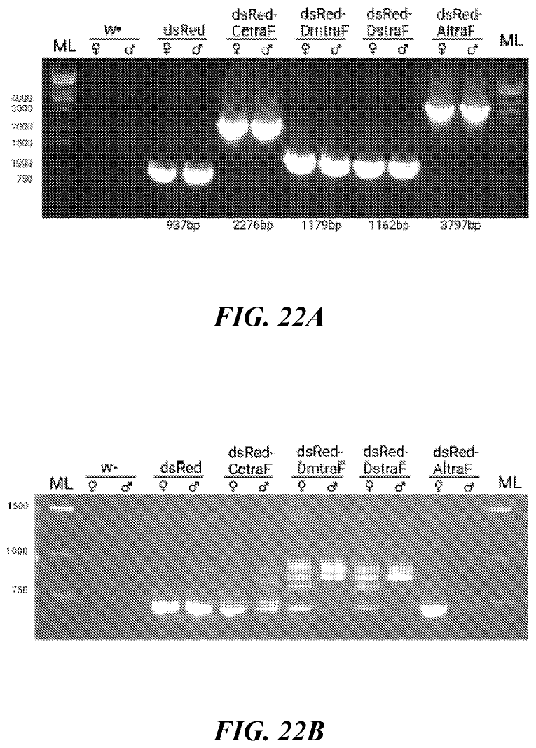

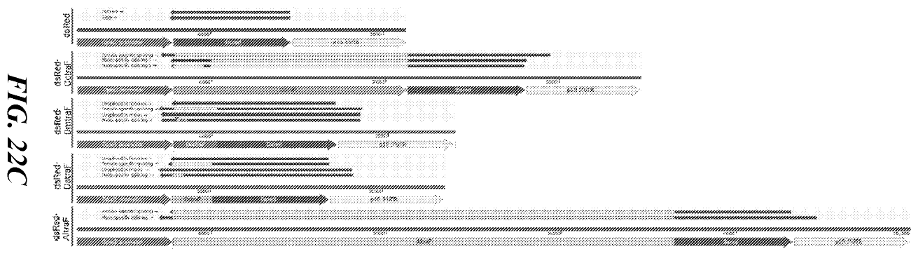

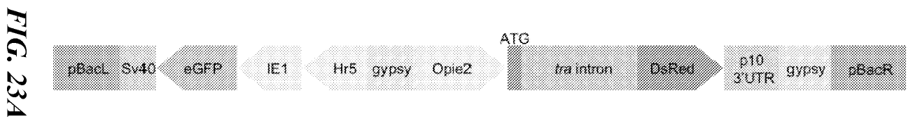

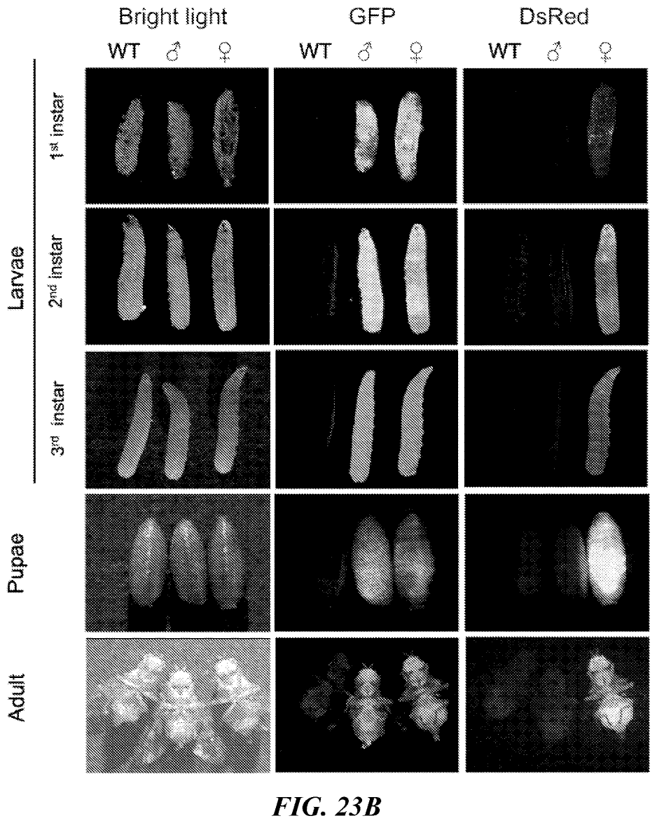

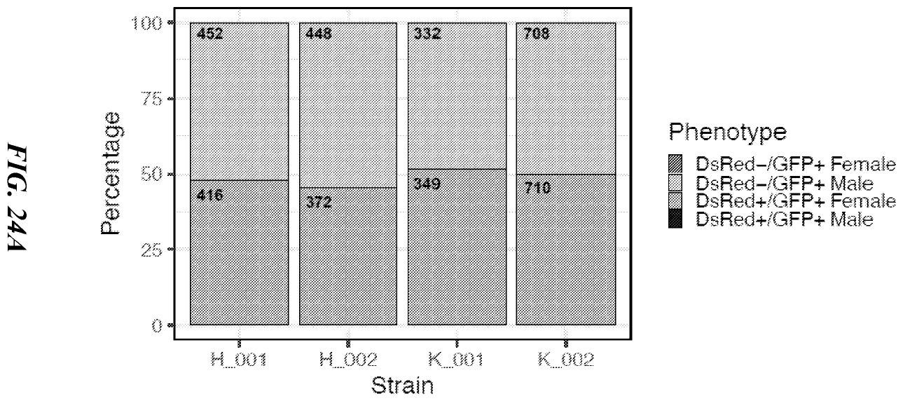

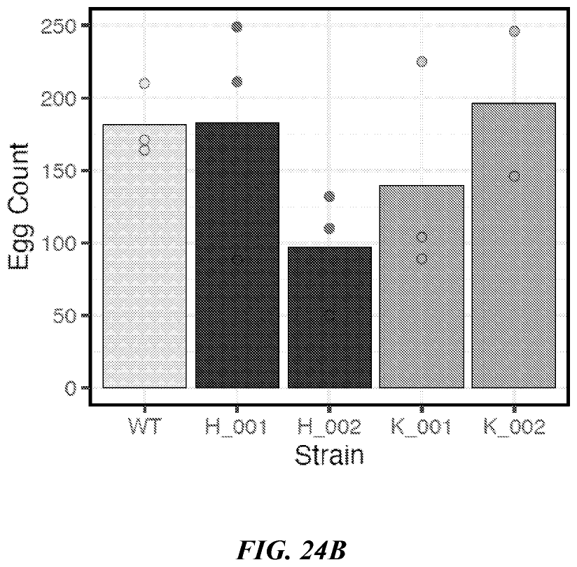

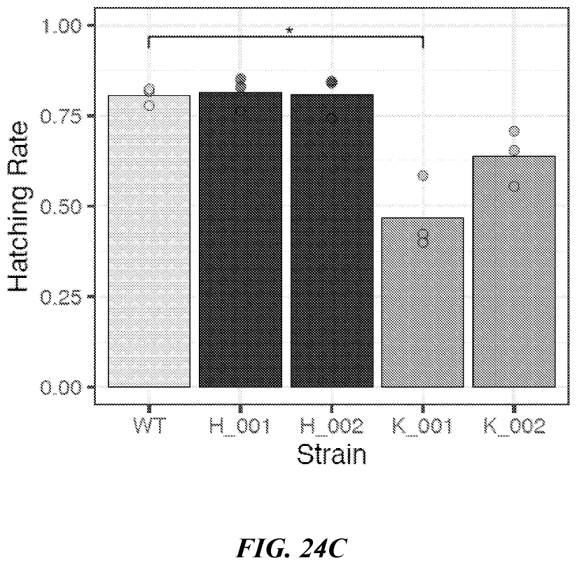

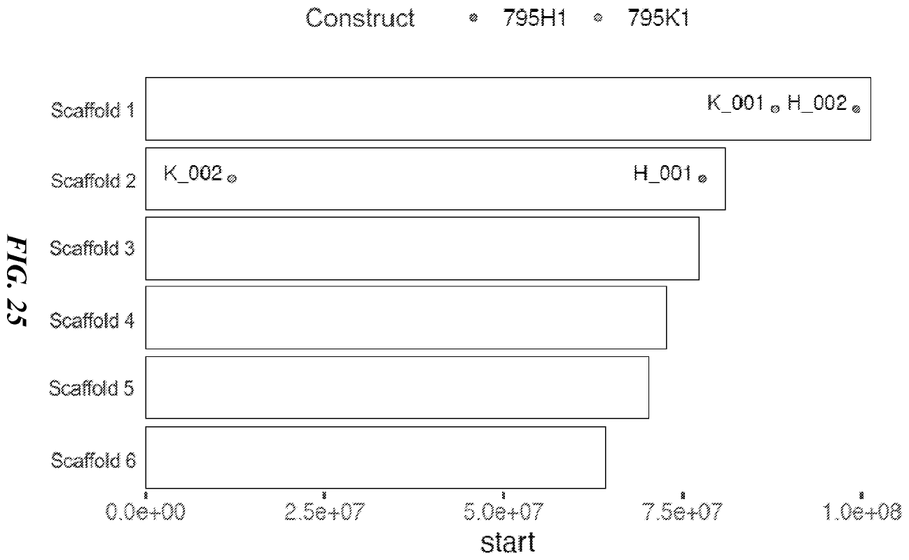

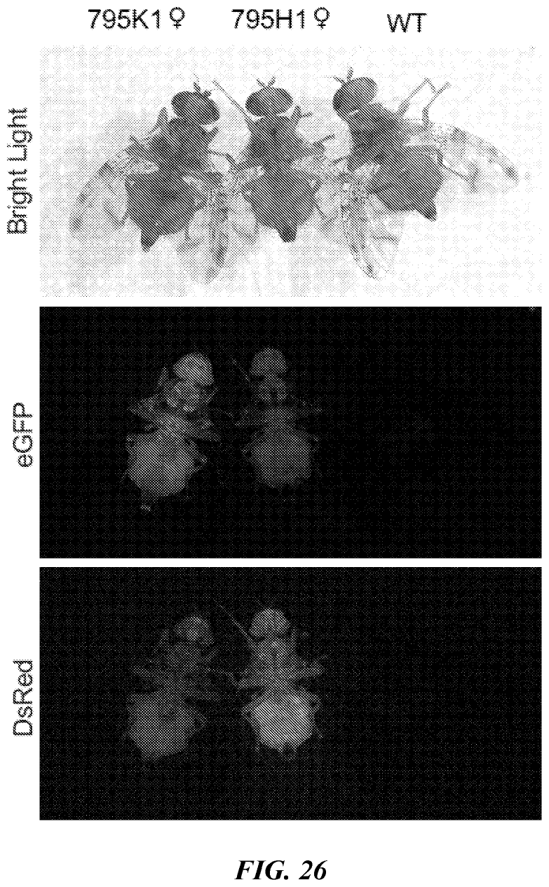

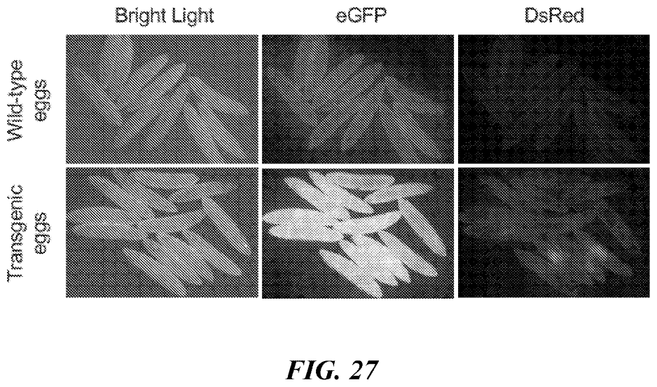

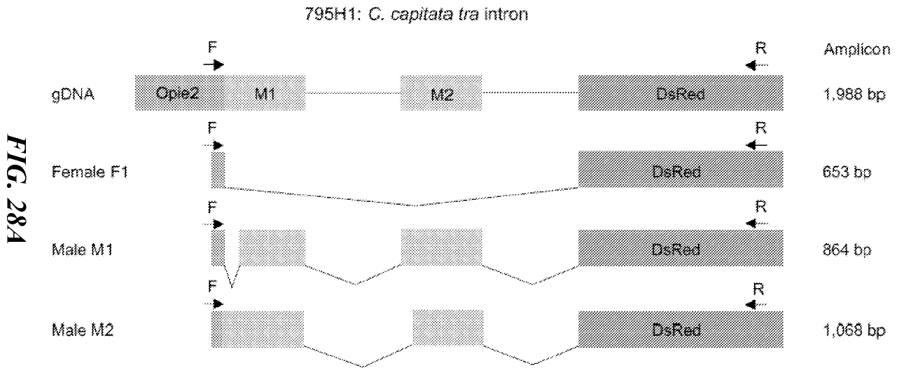

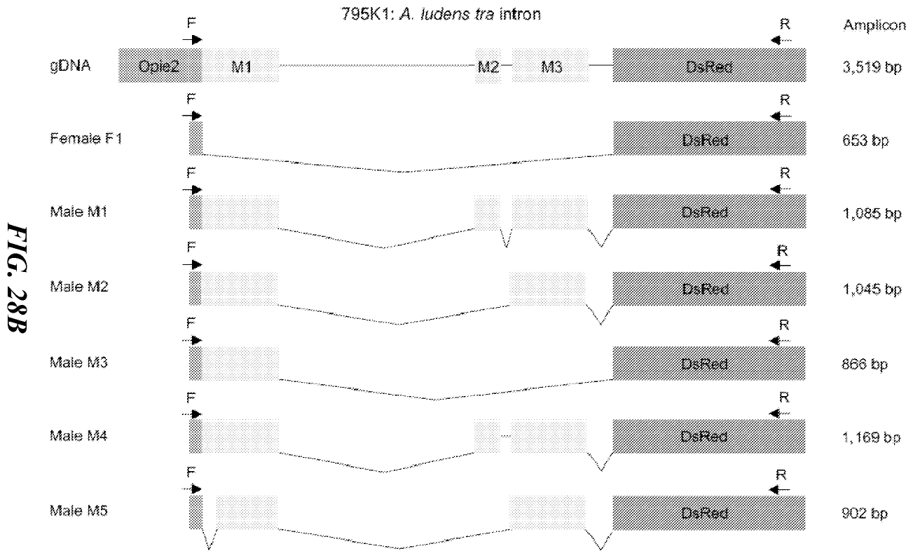

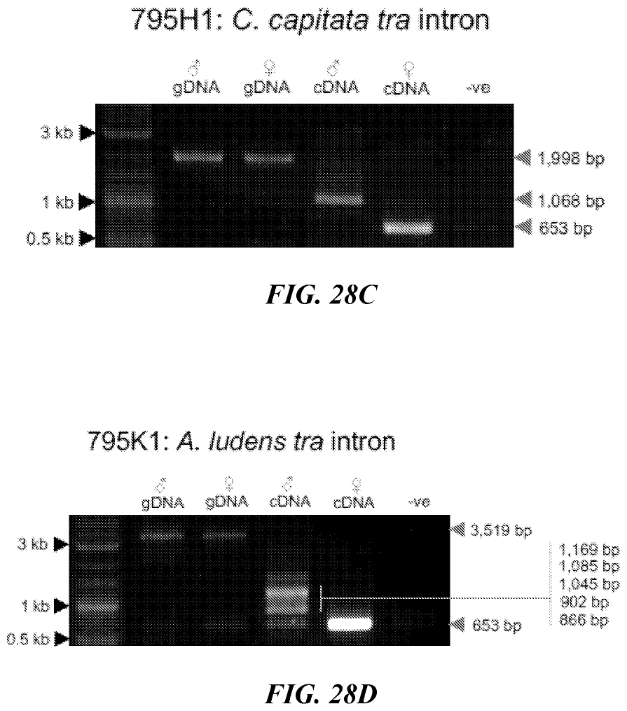

the L1 larval stage using the GFP signal. Following separation, total RNA extraction and RNAseq
analysis were performed. The analysis of the early pupae (EP), mid pupae (MP), and late pupae (LP) stages was conducted using data from a previous study. Sexing at the pupae stages relied on sex-specific morphological differences. Sex-enriched genes were identified using DESeq2 and then performed GO enrichment analysis. The shared genes between the L1 larval stage and all other comparisons were determined, and visualizations such as UpSet plots (FIG. 3A) and Venn diagrams for male mosquitoes (FIG. 3B) and female mosquitoes (FIG. 3C) were created to represent these shared genes.

FIGS. 4A-4B show a process comparison between SEPARATOR and the currently available two-step sex-sorting approaches including radiation-based sterile insect technique (SIT, FIG. 4A) and Wolbachia-based incompatible insect technique (IIT, FIG. 4B). The process of generating radiation-induced sterile male mosquitoes begins with the cultivation of mosquitoes. The larger female pupae are removed using a sieve. However, it should be noted that some smaller female pupae may still remain after the first sex sorting. Once the mosquitoes have been irradiated, emerging adult mosquitoes are screened out by an image recognition AI that has been trained to discriminate between female and male adult morphologies during the second sex sorting. The SEPARATOR approach utilizes a male-specific reporter (GFP) to positively select male L1 larvae. This can be done using the COPAS® instrument, which is capable of high-throughput selection at a speed of up to 10 larvae per second. By removing female larvae early in the development process, SEPARATOR supports a more efficient production of males for SIT application. Furthermore, SEPARATOR allows for the transportation and release of irradiated sex-sorted pupae. This means that adult male mosquitoes can emerge directly into the environment without incurring additional fitness costs from handling and transportation (FIG. 4A). The Wolbachia-based incompatible insect technique (IIT) utilizes a two-step sex-sorting approach for sorting the Wolbachia-infected male mosquitoes currently in use. The process of generating Wolbachia-infected SEPARATOR mosquitoes is relatively simple, as it involves crossing Wolbachia-infected female mosquitoes with SEPARATOR male mosquitoes. The resulting Wolbachia-infected male L1 larvae that express a male-specific reporter (GFP) are then positively selected using the COPAS® instrument (FIG. 4B).

FIG. 5A shows relative locations of the primer target sites the 3′ end of the Hr5Ie1 promoter sequence and the 5′ end of the EGFP coding sequence the construct of SEPARATOR.

FIG. 5B shows the PCR products visualized by gel electrophoresis, with the subsequent validation of the splicing junctions by sequencing. The resulting splicing patterns are depicted in the right panel.

FIG. 5C shows relative levels of non-sex-specifically regulated exons (exon4 and exon6) and female-specific exons (exon5a, exon5b) of SEPARATOR, determined through RNA sequencing (RNAseq) analysis.

FIG. 6 shows sex-specific RNA splicing patterns of SEPARATOR verified through RNA sequencing analysis. The splicing patterns of SEPARATOR were verified through RNAseq analysis in both GFP-positive and GFP-negative mosquitoes, with triple biological replicates for each condition. The RNAseq reads for the different genotypes were aligned, and the location of exons is indicated at the bottom.

FIG. 7 shows coverage distributions of three chromosomes (Chr1, Chr2 and Chr3) and the SEPARATOR transgenes (1174D) in SEPARATOR mosquitoes. The center line represents the median, while the first and third quartiles define the boundaries of the box. The upper and lower whiskers extend from the box to the highest and lowest observed values, respectively, but no further than 1.5 times the Interquartile Range (IQR) from the box. Based on the sequencing depths, the coverage for chromosomes 1, 2, and 3 were 6.31, 6.30, and 6.08, respectively, while the coverage for the SEPARATOR transgenes was 16.14. From the coverage analysis, it suggests that the SEPARATOR transgene (1174D) is present in three copies.

FIG. 8 shows COPAS® data processing with the larvae's size and optical density criteria (Ext/Tof), fluorescence (GFP/RFP), DBSCAN clustering, and the final determination of the GFP-positive larvae.

FIGS. 9A-9B show transcription profiling and expression analysis of GFP-positive and GFP-negative larvae at the L1 stage in SEPARATOR mosquitoes, including PCA analysis (FIG. 9A) and hierarchical clustering of six samples used for RNA sequencing (FIG. 9B).

FIGS. 9C-9E show MA-plots for differential expression patterns between GFP-positive and GFP-negative larvae at the L1 stage in SEPARATOR mosquitoes (FIG. 9C), with additional network visualization of enriched Gene Ontology (GO) terms for the upregulated (FIG. 9D) and the downregulated (FIG. 9E) genes.

FIG. 10A shows transcriptome comparison in GFP-positive (Male, L1M) and GFP− negative (Female, L1F) larvae at the L1 stage from SEPARATOR mosquitoes.

FIG. 10B shows transcriptome comparison between larvae from SEPARATOR mosquitoes, and adult mosquitoes (pupae and carcass) from Matthews's RNA-seq datasets.

FIG. 11 shows identification of male-enriched genes from different developmental stages in the transcriptome comparison analysis. In the transcriptome comparison analysis, L1 stage larvae was included from SEPARATOR mosquitoes. Furthermore, L3 and L4 stage larvae were incorporated, along with early pupae (EP), mid pupae (MP), late pupae (LP), and adult mosquito carcass (Adult) from Matthews's RNA-seq datasets.

FIG. 12 shows identification of female-enriched genes from different developmental stages in the transcriptome comparison analysis. In the transcriptome comparison analysis, L1 stage larvae were included from SEPARATOR mosquitoes. Furthermore, L3 and L4 stage larvae were incorporated, along with early pupae (EP), mid pupae (MP), late pupae (LP), and adult mosquito carcass (Adult) from Matthews's RNA-seq datasets.

FIG. 13 shows the results of gene ontology (GO) analysis on sex-enriched genes throughout various developmental stages. In the transcriptome comparison analysis, L1 stage larvae was included from SEPARATOR mosquitoes. Furthermore, L3 and L4 stage larvae were incorporated, along with early pupae (EP), mid pupae (MP), late pupae (LP), and adult mosquito carcass (Adult) from Matthews's RNA-seq datasets.

FIGS. 14A-14B show identification and isolation a distinct set of genes associated with the larvae stage using gene expression analysis and clustering methods. Through mfuzz clustering analysis using comprehensive developmental stage data, specific genes associated with either L1 or L2-L4 stages were identified. Notably, cluster 17 predominantly consisted of genes expressed in L1 (FIG. 14A), while cluster 1 exhibited gene expression primarily in L2-L4 stages (FIG. 14B).

FIGS. 15A-15B show the schematic maps of vector plasmids coding the sex sorting gene system including a male splicing gene expression system (FIG. 15A) and a two-marker gene expression system (FIG. 15B).

FIGS. 16A-16D show an exemplary sex-sorter cassette in Drosophila. FIG. 16A shows sex-specific alternative splicing and the resulting protein of the transformer (tra) in D. melanogaster, D. suzukii, C. capitata, and A. ludens. FIG. 16B shows splicing of the female-specific transformer (TraF) intron should result in functional dsRed protein in females but not in males. FIG. 16C shows an exemplary schematic of the sex-sorter constructs engineered and tested in the study. TraF introns from D. melanogaster, D. suzukii, C. capitata, and A. ludens are inserted into the coding sequence of either dsRed or eGFP after the ATG translational start codon. FIG. 16D shows fluorescence expression of females and males carrying the respective constructs.

FIGS. 17A-17C show that expression of Opie2-TraF-dsRed or Hr5ie1-TraF-eGFP transgenes in D. melanogaster can be observed in different developmental stages (FIG. 17A) L1-L3 larval stage, (FIG. 17B) pupal stage, (FIG. 17C) adult under white light. RFP and GFP filters.

FIG. 18 shows female selection efficiency at different life stages in D. melanogaster for all six sex-sorter cassettes that give female-specific fluorescence and the numbers of scored flies are indicated for each bar.

FIGS. 19A-19B show fitness cost of all eight sex-sorting cassettes being accessed through two parameters: (FIG. 19A) egg-hatching rate and (FIG. 19B) survival rate to adulthood. Fitness cost was observed in CctraF-dsRed strain in the parameter of survival rate to adulthood (*p<0.05, ***p<0.001, Student's t-test with equal variance.)

FIG. 20A shows a comparison of the transformer female-specific intron splice donor and acceptor sites from D. melanogaster, D. suzukii, C. capitata, and A. ludens. FIG. 20B shows alignment of the sequences at the 5′ beginning (upper panel) and the 3′ end (lower panel) of the intron.

FIG. 21 shows protein alignment of the transformer protein in D. melanogaster, D. suzukii, C. capitata, and A. ludens.

FIGS. 22A-22C show the traF introns splicing patterns in D. melanogaster. Gel electrophoresis

images show (FIG. 22A) the genomic DNA PCR for dsRed-traF (FIG. 22B) the cDNA for the dsRed-traF. ML: molecular ladder. FIG. 22C shows sequencing results of the cDNA from each band.

FIGS. 23A-23B show SEPARATOR system for sex-sorting of Ceratitis capitata. FIG. 23A shows a schematic representation of the Sexing Element Produced by Alternative RNA-splicing of Transgenic Observable reporter (SEPARATOR) cassettes, 795H1 and 795K1. Both cassettes contain two functional elements: constituent Hr5IE1-eGFP-SV40 and Opie2-DsRed-p10, in which the DsRed coding sequence is separated by a transformer (tra) intron. The tra intron in 795H1 and 795K1 originates from Ceratitis capitata and Anastrepha ludens, respectively. FIG. 23B shows that in the SEPARATOR strains the males produce eGFP only, whilst females express both eGFP and DsRed. The representative images of larval, pupal and adult stages of wild-type (WT), and homozygous male and female individuals are compared against each other under bright light, and green fluorescence protein (GFP), and red fluorescence protein (RFP) filters. All images were taken for the H-002 strain harboring the 795H1 construct. Images for all life stages were taken under bright light, and green fluorescence protein (GFP), and red fluorescence protein (RFP) filters.

FIGS. 24A-24C show characterization of transgenic SEPARATOR strains. FIG. 24A shows a stack graph showing the fluorescence phenotype distributions by sex of the four homozygous

transgenic strains at the 9th and 10th generations with over 3,500 adult flies screened. Only the desired DsRed+/GFP+ females and DsRed−/GFP+ males were observed, whilst no DsRed+/GFP+ males or DsRed−/GFP+ females were observed. FIG. 24B shows egg laying rates within strain crosses for all four strains that were compared to wild-type through the egg laying rates within a 5-hour period. FIG. 24C shows egg hatching rate within strain crosses for all four strains were compared to wild-type through the hatching rates of eggs laid within a 5-hour period. FIGS. 24A-24C show H-001 and H-002 strains have the 795H1 cassette harboring the endogenous Ceratitis capitata transformer (tra) intron, while K-001 and K-002 carry the 795K1 cassette with the Anastrepha ludens tra intron. (FIG. 24A) Chi-squared tests showed no statistical significance in sex ratio distortion for any of the four strains. (FIGS. 24B-24C) Dunn test statistical significance was established at follows: p<0.05=* and the wild-type—transgenic strain significance is displayed on the graphs. (FIGS. 24B-24C) The bar levels represent the mean value whilst the dots represent raw values of the replicates. SEPARATOR stands for Sexing Element Produced by Alternative RNA-splicing of a Transgenic Observable reporter.

FIG. 25 shows the map of integrations of the 4 unique strains for the 795H1 and 795K1 constructs. H-001 and H-002 strains harbor the 795H1 cassette, while K-001 and K-002 strains harbor the 795K1 cassette.

FIG. 26 shows images of 795H1 and 795K1 homozygous females, wherein the homozygous females harboring the Anastrepha ludens transformer (tra) intron-containing 795K1 cassette have a weaker DsRed signal (left) compared to homozygous females harboring the Ceratitis capitata tra intron containing 795H1 cassette (middle). These were imaged alongside a wild-type female (right).

FIG. 27 shows images of transgenic and wild-type eggs, wherein the egg images showcase the wild-type eggs, and the homozygous SEPARATOR eggs, all expressing GFP and a variable degree of DsRed.

FIGS. 28A-28D show diagrams showcasing the expected sex-specific DsRed splicing patterns in (FIG. 28A) the 795H1-harboring and (FIG. 28B) 795K1-harboring flies via the transformer (tra) intron from (FIG. 28A) Ceratitis capitata and (FIG. 28B) Anastrepha ludens accordingly. Forward and reverse primers, specific to the exogenous elements of both constructs, used in the PCR amplification are shown in (FIG. 28A) and (FIG. 28B) as F and R, respectively. FIGS. 28C-28D are annotated electrophoresis gel images with amplification from male and female genomic DNA (gDNA), and male, and female cDNA. A DNA ladder was run in the left-most

lanes and negative controls in the right-most lanes.

### DETAILED DESCRIPTION

Provided herein are methods of sex-sorting a plurality of animals (e.g., insects) based on sex-specific gene expression, including (a) generating an exogenous nucleic acid molecule; (b) delivering the exogenous nucleic acid molecule into an insect from the plurality of insects, where the exogenous nucleic acid molecule includes a promoter region, a sex-specific splicing module, a reporter gene, and a transcription terminator; (c) detecting sex-specific gene expression of the reporter gene; and (d) sorting the insect from the plurality of insects based on the detecting of the sex-specific gene expression in step (c).

Also provided herein are methods of identifying a sex of an insect based on sex-specific gene expression, including (a) generating an exogenous nucleic acid molecule; (b) delivering the exogenous nucleic acid molecule into an insect, wherein the exogenous nucleic acid molecule comprises a promoter region, a sex-specific splicing module, a reporter gene, and a transcription terminator; and (c) identifying sex-specific gene expression of the reporter gene.

It is noted that as used in the specification and the appended claims, the singular forms “a”, “an” and “the” refer to one or more (i.e., at least one) of the grammatical object of the article unless the context clearly dictates otherwise. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelle biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein. “engineered” or “genetically engineered,” in reference to organisms (e.g., insects), refers to an organism that comprises a nucleic acid sequence (e.g., DNA, RNA, or mRNA) that is not present in, or is present at a different level than, an otherwise similar organisms under similar conditions that is not engineered (an exogenous nucleic acid), or an organism that comprises a polypeptide expressed from said nucleic acid. In some embodiments, a genetically engineered organism has been altered from its native state by the introduction of an exogenous nucleic acid, or is the progeny of such an altered organism. In some embodiments, a genetically engineered organism comprises an exogenous nucleic acid (e.g., DNA, RNA, or mRNA).

As used herein, the term “endogenous” refers to any substances and processes that originate from within a living system such as an organism, tissue, or cell.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the terms “nucleic acid” and “nucleotide” are intended to be consistent with their use in the art and to include naturally-occurring species or functional analogs thereof. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

### Methods of Sex Sorting and Identifying the Sex of an Insect

Provided herein are methods of sex-sorting in an insect based on sex-specific gene expression including (a) generating an exogenous nucleic acid molecule; (b) delivering the exogenous nucleic acid molecule into an insect from the plurality of insects, where the exogenous nucleic acid molecule includes a promoter region, a sex-specific splicing module, a reporter gene, and a transcription terminator; (c) detecting sex-specific gene expression of the reporter gene; and (d) sorting the insect from the plurality of insects based on the detecting of the sex-specific gene expression in step (c), thereby sex-sorting the insect based on the sex-specific gene expression.

Also provided herein are methods of identifying the sex of an insect based on sex-specific gene expression, including (a) generating an exogenous nucleic acid molecule; (b) delivering the exogenous nucleic acid molecule into an insect, wherein the exogenous nucleic acid molecule comprises a promoter region, a sex-specific splicing module, a reporter gene, and a transcription terminator; and (c) identifying sex-specific gene expression of the reporter gene, thereby identifying the sex of the insect based on the sex-specific gene expression.

As used herein, an “insect” can refer to any member of the largest class of the phylum Arthropoda, which is itself the largest of the animal phyla. Insects have segmented bodies, jointed legs, and external skeletons (e.g., exoskeletons). In some embodiments, an insect can include a bedbug, a housefly, a clothes moth, a Japanese beetle, an aphid, a mosquito, a flea, a horsefly, a homet, a butterfly, or a moth. In some embodiments, an insect can be a mosquito from the genera Stegomyia, Aedes, Anopheles, or Culex. In some embodiments, the mosquito can include Aedes aegypti, Aedes albopictus, Ochlerotatus triseriatus (Aedes triseriatus). Anopheles stephensi, Anopheles albimanus, Anopheles gambiae. Anopheles quadrimaculatus, Anopheles freeborni, Culex species, or Culiseta melanura. In some embodiments, the insect can include a tephritid fruit fly selected from Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata), Caribbean fruit fly (Anastrepha suspensa), Oriental Fruit Fly (Bactrocera dorsalis), West Indian fruit fly (Anastrepha obliqua), the New World screwworm (Cochliomyia hominivorax), the Old World screwworm (Chrysomya bezziana), Australian sheep blowfly/greenbottle fly (Lucilia cuprina), the pink bollworm (Pectinophora gossypiella), the European Gypsy moth (Lymantria dispar), the Navel Orange Worm (Amyelois transitella), the Peach Twig Borer (Anarsia lineatella), the rice stem borer (Tryporyza incertulas), the noctuid moths, Heliothinae, the Japanese beetle (Papilla japonica), White-fringed beetle (Graphognatus spp.). Boll weevil (Anthonomous grandis), the Colorado potato beetle (Leptinotarsa decern lineata), the vine mealybug (Planococcus ficus). Asian citrus psyllid (Diaphorina citri), Spotted wing Drosophila (Drosophila suzukii), Bluegreen sharpshooter (Graphocephala atropunctata), Glassy winged sharpshooter (Flornalodisca vitripennis), Light brown apple moth (Epiphyas postvittana), Bagrada bug (Bagrada hilaris), Brown marmorated stink bug (Halyomorpha halys), Asian Gypsy Moth selected from the group of Lymantria dispar asiatica, Lymantria dispar japonica, Lymantria albescens, Lymantria umbrosa, and Lymantria postalba, Asian longhorned beetle (Anoplophora glabripennis), Coconut Rhinoceros Beetle (Oryctes rhinoceros), Emerald Ash Borer (Agrilus planipennis). European Grapevine Moth (Lobesia botrana), European Gypsy Moth (Lymantria dispar), False Codling Moth (Thaumatotibia leucotreta), fire ants selected from Solenopsis invicta Buren, and S. richteri Forel, Old World Bollworm (Flelicoverpa armigera), Spotted Lanternfly (Lycorma delicatula), Africanized honeybee (Apis mellifera scutellata), Fruit and shoot borer (Leucinodes orbonalis), corn root worm (Diabrotica spp.). Western corn rootworm (Diabrotica virgifera), Whitefly (Bemisia tabaci), Flouse Fly (Musca domestica), Green Bottle Fly (Lucilia cuprina), Silk Moth (Bombyx mori), Red Scale (Aonidiella aurantia), Dog heartworm (Dirofilaria immitis), Southern pine beetle (Dendroctonus frontalis), Avocado thrip (Thysanoptera Spp.), Botfly selected from Oestridae spp. and Dermatobia hominis), Florse Fly (Tabanus sulcifrons), Florn Fly (Flaematobia irritans), Screwworm Fly selected from Cochliomyia macellaria (C. macellaria), C. hominivorax, C. aldrichi, or C. minima, Tsetse Fly (Glossina spp.), Warble Fly selected from Flypoderma bovis or Hypoderma lineatum, Spotted lanternfly (Lycorma delicatula), Khapra beetle (Trogoderma granarium), Honeybee mite (Varroa destructor), Termites (Coptotermes formosanus). Hemlock woolly adelgid (Adelges tsugae), Walnut twig beetle (Pityophthorus juglandis), European wood wasp (Sirex noctilio), Pink-spotted bollworm (Pectinophora scutigera), Two spotted spider mite (Tertanychus urticae), Diamondback moth (Plutella xylostella), Taro caterpillar (Spodoptera litura), Red flour beetle (Tribolium castaneum), Green peach aphid (Myzus persicae). Cotton Aphid (Aphis gossypii), Brown planthopper (Nilaparvata lugens), Beet armyworm (Spodotera exigua), Western flower thrips (Frankliniella occidentalis), Codling moth (Cydia pomonella), Cowpea weevil (Callosobruchus maculatus), Pea aphid (Acyrthosiphon pisum), Tomato leafminer (Tuta absoluta), Onion thrips (Thrips tabaci), or Cotton bollworm (Helicoverpa armigera). In some embodiments, the insect is Aedes aegypti. In some embodiments, the insect is Drosophila melanogaster. In some embodiments, the insect is Drosophila suzukii. In some embodiments, the insect is Ceratitis capitata. In some embodiments, the insect is Anastrepha ludens. See, e.g., Davydova et al., doi.org/10.1101/2023.09.29.560088, 2023; Liu et al., bioRxiv. 2023 Aug. 14:2023.08.11.553026; and Weng et al., bioRxiv. 2023 Jul. 12:2023.06.16.545348, which are herein incorporated by reference in their entireties.

In some embodiments, any one of the methods described herein can exploit a sex-specific expression via sex-specific alternative splicing (SSAS) of a reporter gene. In some embodiments, any one of the methods described herein can exploit male specific expression via sex-specific alternative splicing (SSAS) of a reporter gene. In some embodiments, any one of the methods described herein can exploit female specific expression via sex-specific alternative splicing (SSAS) of a reporter gene. In some embodiments, sex-sorting of an insect and/or identifying a sex of an insect can identify the insect as a male. In some embodiments, sex-sorting of an insect and/or identifying a sex of an insect can identify the insect as a female.

The term “RNA splicing” refers to a process in molecular biology where a newly-made precursor messenger RNA (pre-mRNA) transcript is transformed into a mature messenger RNA (mRNA). It works by removing all the introns (non-coding regions of RNA) and splicing back together exons (coding regions). For nuclear-encoded genes, splicing occurs in the nucleus either during or immediately after transcription. For those eukaryotic genes that contain introns, splicing is usually needed to create an mRNA molecule that can be translated into protein. For many eukaryotic introns, splicing occurs in a series of reactions which are catalyzed by the spliceosome, a complex of small nuclear ribonucleoproteins (snRNPs). The term “alternative splicing” refers to a cellular process in which exons from the same gene are joined in different combinations, leading to different, but related, mRNA transcripts. The resulting mRNAs can be translated to produce different proteins with distinct structures and functions, while being derived from a same parent gene.

In some embodiments, sex-specific expression of a reporter gene comprises sex-specific alternative splicing of the reporter gene, wherein the reporter gene is an innocuous fluorescent marker. In some embodiments, the reporter gene comprises a DsRed gene, an EGFP gene, or any combinations thereof.

In some embodiments, any one of the methods described herein can enable sex-sorting of an insect during early larval development. In some embodiments, any one of the methods described herein can enable sex-sorting of an insect as a mature embryo. In some embodiments, any one of the methods described herein can be adaptable for high-throughput sorting. In some embodiments, high-throughput sorting comprises sorting of insects at a speed of up to 740 larvae/minute (e.g., up to 300 larvae/minute, up to 400 larvae/minute, up to 500 larvae/minute, up to 600 larvae/minute, or up to 700 larvae/minute).

In some embodiments, any one of the methods described herein can enable sex-sorting of an insect without relying on sex-chromosome linkage. In some embodiments, any one of the methods described herein comprises a sex-specific gene expression system that is genetically stable and not prone to breakage by meiotic recombination or chromosomal rearrangement. In some embodiments, any one of the methods described herein is portable to alternate species as it utilizes transposable elements, promoters, and markers that are cross-species portable.

In some embodiments, any one of the methods described herein can be referred to as Sexing Element Produced by Alternative RNA-splicing of a Transgenic Observable Reporter (“SEPARATOR”).

In some embodiments, any one of the methods described herein can be used for high-throughput sex-selection in Aedes mosquitoes or other insects. In some embodiments, any one of the methods described herein can be a positive-selection system. In some embodiments, any one of the methods described herein can positively select male mosquito L1 larvae. In some embodiments, any one of the methods described herein can positively select a male L1 larvae that expresses a dominant male-specific reporter gene (e.g., EGFP). In some embodiments, any one of the methods described herein does not rely on morphological differences between male and female mosquitoes during their pupal and adult stages to differentiate between the two sexes. In some embodiments, any one of the methods described herein does not require distinguishing size difference between female and male pupae.

In some embodiments, any one of the methods described herein can automate the process on a large scale. In some embodiments, any one of the methods described herein does not require a significant amount of human effort and time. In some embodiments, any one of the methods described herein can be portable across multiple species.

In some embodiments, any one of the methods described herein comprises sex-sorting a plurality of insects based on sex-specific gene expression. As used herein, the term “sex-sorting” refers to sorting and separating a plurality of insects (e.g., Aedes aegypti, Drosophila melanogaster, Drosophila suzukii, Ceratitis capitata, or Anastrepha ludens) into two groups (e.g., male and female). In some embodiments, an insect of a plurality of insects can be identified as a male insect. In some embodiments, an insect of a plurality of insects can be identified as a female insect. In some embodiments, an insect can be removed from the plurality of insects once it is identified as a male insect. In some embodiments, an insect can be removed from the plurality of insects once it is identified as a female insect. In some embodiments, sex-sorting can include sorting insects at the larval stage by fluorescence and separating the larvae into two groups (e.g., EGFP-positive and EGFP-negative). In some embodiments, sex-sorting can include automated sex sorting, wherein fluorescence-based flow cytometry is used to sort batches of larvae (e.g., several thousand larvae), and wherein male larvae expressing a reporter gene (e.g., EGFP) can form a distinct cluster and be clearly separated from female larvae that do not express the reporter gene. In some embodiments, sex-sorting can include sorting insects at the larval stage by fluorescence by performing various methods known in the art. For example, such methods can include, but are not limited to, fluorescence-activated cell sorting (FACS), flow cytometry, automated fluorescence microscopy, microfluidics combined with fluorescence-base assays, or fluorescence microplate readers. See, e.g., Marois et al., Malar J. 2012 Aug. 28:11:302; Midkiff et al., Molecules. 2019 December; 24(23): 4292, which are herein incorporated by reference in their entireties.

In some embodiments, sex-sorting can further include sorting a plurality of insects according to a distinct size difference between female and male insects. In some embodiments, a female insect can be larger in size compared to a male insect. In some embodiments, sex-sorting can include sorting a plurality of insects to be size-sorted through a sieve. In some embodiments, sex-sorting can further include utilizing visual cues from sexual dimorphic differences in insect morphology to identify and separate an insect from the plurality of insects.

In some embodiments, any one of the methods described herein comprises detecting and/or identifying sex-specific gene expression. In some embodiments, detecting and/or identifying sex-specific gene expression can include using RNA sequencing to identify genes exhibiting sex-specific expression patterns. In some embodiments, detecting and/or identifying sex-specific gene expression can further include conducting a comprehensive analysis of differential gene expression (DGE), wherein specific genes can be found to be differentially expressed according to the sex and/or the developmental stage of the insect. In some embodiments, a sex-specific gene can exhibit male-enriched expression patterns at the early L1 larvae stage of an insect. In some embodiments, a sex-specific gene can exhibit female-enriched expression patterns at the early L1 larvae stage of an insect. In some embodiments, RNA sequencing can be performed at the L1 larvae stage of an insect. In some embodiments, RNA sequencing can be performed at the L3 larvae stage of an insect. In some embodiments, RNA sequencing can be performed at the L4 larvae stage of an insect. In some embodiments, RNA sequencing can be performed at the pupae stage of an insect. In some embodiments, RNA sequencing can be performed at the adult stage of an insect.

In some embodiments, any one of the methods described herein can detect fluorescence reporter genes (e.g., EGFP and DsRed) by a fluorescence detector within any type of flow cytometers or sorters. In some embodiments, any one of the methods described herein comprises a sorter to collect the selected insects. In some embodiments, any one of the methods described herein comprises using a Complex Object Parametric Analyzer and Sorter (COPAS®) that allows scalable high-throughput sex-selection of insects.

### Exogenous Nucleic Acid Molecules

Provided herein are exogenous nucleic acid molecules that include a promoter region, a sex-specific splicing module, a reporter gene, and a transcription terminator.

In some embodiments of any of the exogenous nucleic acid molecules described herein, the exogenous nucleic acid molecule includes a promoter region (e.g., any of the exemplary promoters described herein). As used herein, the term “promoter” may refer to a DNA sequence recognized by enzymes/proteins in a mammalian cell required to initiate the transcription of an linked coding sequence. A promoter typically refers to e.g., a nucleotide sequence to which an RNA polymerase and/or any associated factor binds and at which transcription is initiated. The promoter can be constitutive, inducible, or tissue-specific (e.g., a brain-specific promoter). The promoter can be an exogenous promoter operably linked to an isolated nucleic acid. The promoter can also be a genomic sequence where the promoter is proximal to a transcription start site and at least partially controls expression of the associated gene product. A promoter within the genome can be either proximal (e.g., within 2000 nucleotides) or distal (e.g., greater than 2000 nucleotides) from a transcription start site. Non-limiting exemplary promoters include CMV, CBA, CAG, Cbh, EF-1α, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. See, e.g., U.S. Pat. No. 11,512,327, which is incorporated by reference in its entirety. In some embodiments, the promoter region can include a Hr5IE1 promoter. In some embodiments, the promoter region can include an OpIE-1 promoter.

In some embodiments of any of the exogenous nucleic acid molecules described herein, an exogenous nucleic acid molecule includes a sex-specific splicing module. In some embodiments, the sex-specific splicing module can include an endogenous sex-specific exonic sequence and a truncated intronic sequence. In some embodiments, the sex-specific splicing module is a male-specific splicing module. In some embodiments, the male-specific splicing module comprises exon 6, exon 6, or any combinations thereof. In some embodiments, the sex-specific splicing module is a female-specific splicing module. In some embodiments, the female-specific splicing module comprises exon 4, exon 5b, exon 6, or any combinations thereof. In some embodiments, the female-specific splicing module comprises an engineered exon 5b, wherein the engineered exon 5B is engineered to exclude one or more stop codons from the exon 5b. In some embodiments, the sex-specific splicing module is derived from Ae. aegypti doublesex (AaeDsx). In some embodiments, the sex-specific splicing module is derived from C. capitata transformer (traF). In some embodiments, the sex-specific splicing module is derived from D. melanogaster traF. In some embodiments, the sex-specific splicing module is derived from D. suzukii traF.

In some embodiments of any of the exogenous nucleic acid molecules described herein, an exogenous nucleic acid molecule includes a reporter gene. As used herein, the term “reporter gene”, often simply “reporter”, refers to a gene that researchers attach to a regulatory sequence of another gene of interest in bacteria, cell culture, animals, or plants. Such genes are called reporters because the characteristics they confer on organisms expressing them are easily identified and measured, or because they are selectable markers. Reporter genes are often used as an indication of whether a certain gene has been taken up by or expressed in the cell or organism population. Commonly used reporter genes that induce visually identifiable characteristics usually involve fluorescent and luminescent proteins. Examples of a reporter gene can include, but are not limited to, the genes encoding fluorescent proteins (e.g., GFP, dsRed, YFP, RFP, mCherry, and EGFP), luciferase (e.g., firefly luciferase, renilla luciferase). β-Galactosidase (e.g., LacZ,), HaloTag. and GUS (β-Glucuronidase). (Lippincott-Schwartz and Patterson, Sci. 300: 87-91; Contag and Bachmann, Annu. Rev. Biomed. Eng. 4: 235-260; Salehi et al, Hum. Gene Ther. 20 (1):21-30; Giepmans et al, Sci. 312 (5771): 217-224; Marathe and McEwen, Gene. 154 (1): 105-107) In some embodiments, a reporter gene comprises a DsRed gene, an EGFP gene, or any combinations thereof.

In some embodiments, an exogenous nucleic acid molecule comprises a coding sequence for one or more reporter genes. In some embodiments, an exogenous nucleic acid molecule comprises coding sequences for the one or more reporter genes such that the reporter gene can be expressed in a sex-specific manner. In some embodiments, an exogenous nucleic acid molecule includes a coding sequence for EGFP and DsRed, wherein EGFP and DsRed is expressed in a sex-specific manner. In some embodiments, an exogenous nucleic acid molecule comprises a DsRed coding sequence that is in-frame with a female-specific splicing module (exon4, engineered exon5b, and exon6), thereby controlling female-specific DsRed expression. In some embodiments, an exogenous nucleic acid molecule comprises a EGFP coding sequence that is in-frame with a male-specific product (exon4 and exon6), thereby controlling male-specific EGFP expression.

In some embodiments, any one of the methods described herein identify a sex of an insect based on the expression of a sex-specific gene. In some embodiments, an insect can be sorted as male based on a male-specific gene expression. In some embodiments, an insect can be sorted as male based on the expression of the EGFP gene. In some embodiments, an insect can be sorted as female based on a female-specific gene expression. In some embodiments, an insect can be sorted as female based on the expression of the DsRed gene.

In some embodiments, an exogenous nucleic acid molecule comprises a transcription terminator. As used herein, the term “transcription terminator” is a section of nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex. In some embodiments, an exogenous nucleic acid molecule comprises a transcription terminator, wherein the transcription terminator comprises a SV40 poly(A) signal.

In some embodiments, any one of the methods described herein includes a delivering step comprising integrating the exogenous nucleic acid molecule into the genome of the insect. In some embodiments, integrating an exogenous nucleic acid molecule into the genome of the insect is facilitated by a vector.

### Gene Delivery Vectors

In some embodiments of any of the methods described herein, introducing an exogenous nucleic acid molecule into the genome of an insect is facilitated by a vector. For example, a vector can be an expression vector where the expression vector includes a promoter sequence operably linked to the sequence encoding the molecule (e.g., a nucleic acid molecule). Non-limiting examples of vectors include plasmids, transposons (e.g., DNA transposons, RNA transposons or retrotransposons, and class III transposons), cosmids, and viral derived vectors (e.g., any adenoviral derived vectors (AV) cytomegaloviral derived (CMV) vectors, simian viral derived (SV40) vectors, adeno-associated virus (AAV) vectors, lentivirus vectors, and retroviral vectors), and any Gateway® vectors. A vector can, for example, include sufficient cis-acting elements for expression where other elements for expression can be supplied by the host mammalian cell or in an in vitro expression system. Skilled practitioners will be capable of selecting suitable vectors and mammalian cells for introducing any of spatial profiling reagents described herein.

In some embodiments, retrovirus vectors and adeno-associated virus vectors can be used as a recombinant gene delivery system for the transfer of an exogenous nucleic acid molecule. These vectors provide efficient delivery of genes into cells, and the transferred nucleic acids are stably integrated into the chromosomal DNA of the host cell. Protocols for producing recombinant retroviruses and for infecting cells in vitro with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro (see for example Eglitis, et al. (1985) Science 230:1395-1398; Danos and Mulligan (1988) Proc. Natl. Acad. Sci. USA 85:6460-6464: Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644: Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

In some embodiments, another viral gene delivery system useful in the present methods can utilize adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

In some embodiments, helper-dependent (HDAd) vectors can also be produced with all adenoviral sequences deleted except the origin of DNA replication at each end of the viral DNA along with packaging signal at 5-prime end of the genome downstream of the left packaging signal. HDAd vectors are constructed and propagated in the presence of a replication-competent helper adenovirus that provides the required early and late proteins necessary for replication.

In some embodiments, another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. It is also one of the few viruses that may integrate its DNA into non-dividing cells and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective, easily produced, approved by FDA, and tested in multiple clinical trials, paves new avenues for therapeutic gene editing.

In some embodiments, a vector (e.g., gene delivery vector) can include any of the exogenous nucleic acid molecules described herein. In some embodiments, the gene delivery vectors are transposons, where the transposons include DNA transposons, RNA transposons or retrotransposons, and class III transposons. Examples of DNA transposons include Tc1/nariner, piggyBac, hAT, and Helitron. DNA transposons are generally described in, e.g., Wicker et al., Nat. Rev. Genet. 8 (12): 973-982; Feschotte et all., Annu. Rev. Genet. 41 (1): 331-368; and Munoz-Lopez et al., Curr. Genomics. 11 (2): 115-128. RNA transposons, also known as retrotransposons, are mainly classified as long terminal repeats (LTRs) and non-long terminal repeats (non-LTRs). RNA transposons are generally described in, e.g., Finnegan. Curr. Biol. 22 (11): R432-R437; Dombroski et al., Mol. Cel. Biol. 14 (7): 4485-92; and Sanchez et al., Nat. Commun. 8 (1): 1283. Examples of Class III transposons include the Foldback (FB) elements of Drosophila melanogaster, the TU elements of Strongylocentrotus purpuratus, and Miniature Inverted-repeat Transposable Elements. Class III transposons are generally described in, e.g., Boutanaev and Osbourn, PNAS. 115 (28): E6650-E6658; Kaminker et al., Geno. Biol. 3 (12): research0084. In some embodiments, gene delivery vector is piggyBac transposon.

As used herein, the term “piggyBac” refers to PiggyBac (PB) transposon, a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and moves the contents from the original sites and integrates them into TTAA chromosomal sites. The activity of the PiggyBac transposon system enables genes of interest between the two ITRs in the PB vector to be easily mobilized into target genomes. In some embodiments of any of the methods described herein, the exogenous nucleic acid molecule can further include a piggyBac inverted terminal repeat located at each end of the effector region.

As used herein, an “effector region” or “effector domain” can refer to a protein interaction region/domain that can function in transcriptional regulation via their ability to (i) interact with the basal transcriptional machinery and general co-activators, (ii) interact with other transcriptional factors to allow cooperative binding, and (iii) directly or indirectly recruit histone and chromatin modifying enzymes.

In some embodiments of any of the methods described herein, introducing an exogenous nucleic acid molecule into the genome of an insect is facilitated by a vector, wherein the vector comprises SEQ ID NO: 1. In some embodiments of any of the methods described herein, introducing an exogenous nucleic acid molecule into the genome of an insect is facilitated by a vector, wherein the vector comprises SEQ ID NO: 2.

Plasmid sequence of vector 1174D
SEQ ID NO: 1
ttgagatcctttttttctgcgcgtaatctgctgcttgcaaacaaaaaaaccaccgctacc
agcggtggtttgtttgccggatcaagagctaccaactctttttccgaaggtaactggctt
cagcagagcgcagataccaaatactgttcttctagtgtagccgtagttaggccaccactt
caagaactctgtagcaccgcctacatacctcgctctgctaatcctgttaccagtggctgc
tgccagtggcgataagtcgtgtcttaccgggttggactcaagacgatagttaccggataa
ggcgcagcggtcgggctgaacggggggttcgtgcacacagcccagcttggagcgaacgac
ctacaccgaactgagatacctacagcgtgagctatgagaaagcgccacgcttcccgaagg
gagaaaggcggacaggtatccggtaagcggcagggtcggaacaggagagcgcacgaggga
gcttccagggggaaacgcctggtatctttatagtcctgtcgggtttcgccacctctgact
tgagcgtcgatttttgtgatgctcgtcaggggggcggagcctatggaaaaacgccagcaa
cgcggcctttttacggttcctggccttttgctggccttttgctcacatgttctttcctgc
gttatcccctgattctgtggataaccgtattaccgcctttgagtgagctgataccgctcg
ccgcagccgaacgaccgagcgcagcgagtcagtgagcgaggaagcggaagagcgcccaat
acgcaaaccgcctctccccgcgcgttggccgattcattaatgcagctggcacgacaggtt
tcccgactggaaagcgggcagtgagcgcaacgcaattaatgtgagttagctcactcatta
ggcaccccaggctttacactttatgcttccggctcgtatgttgtgtggaattgtgagcgg
ataacaatttcacacaggaaacagctatgaccatgattacgccaagctcgaaattaaccc
tcactaaagggattaaccctagaaagatagtctgcgtaaaattgacgcatgcattcttga
aatattgctctctctttctaaatagcgcgaatccgtcgctgtgcatttaggacatctcag
tcgccgcttggagctcccgtgaggcgtgcttgtcaatgcggtaagtgtcactgattttga
actataacgaccgcgtgagtcaaaatgacgcatgattatcttttacgtgacttttaagat
ttaactcatacgataattatattgttatttcatgttctacttacgtgataacttattata
tatatattttcttgttatagatatctgtcgacatgcccgccgtgaccgtcgagaacccgc
tgacgctgccccgcgtatccgcacccgccgacgccgtcgcacgtcccgtgctcaccgtga
ccaccgcgcccagcggtttcgagggcgagggcttcccggtgcgccgcgcgttcgccggga
tcaactaccgccacctcgacccgttcatcatgatggaccagatgggtgaggtggagtacg
cgcccggggagcccaagggcacgccctggcacccgcaccgcggcttcgagaccgtgacct
acatcgtcgacgggcccgcgtgcaaaagcgtgtttttttgcagtgcaaaaaagttggtgg
tggggaggccaccgagtatgccgcggaattgatcggctaaatggtatggcaagaaaaggt
atgcaatataataatcttttattgggtatgcaacgaaaatttgtttcgtcaacgtatgca
atattttttattaaaagagggtatgcaatgtattttattaaaaacgggtatgcaatataa
taatcttttattgggtatgcaacgaaaatttgtttcgtcaaagtatgcaatattttttat
taaaagagggtatgcaatgtattttattaaaaacgggtatgcaataaaaaattatttggt
ttctctaaaaagtatgcagcacttattttttgataaggtatgcaacaaaattttactttg
ccgaaaatatgcaatgtttttgcgaataaattcaacgcacacttattacgtgaggtaccg
cgtaaaacacaatcaagtatgagtcataagctgatgtcatgttttgcacacggctcataa
ccgaactggctttacgagtagaattctacttgtaacgcacgatcagtggatgatgtcatt
tgtttttcaaatcgagatgatgtcatgttttgcacacggctcataaactcgctttacgag
tagaattctacgtgtaacgcacgatcgattgatgagtcatttgttttgcaatatgatatc
atacaatatgactcatttgtttttcaaaaccgaacttgatttacgggtagaattctactt
gtaaagcacaatcaaaaagatgatgtcatttgtttttcaaaactgaactcgctttacgag
tagaattctacgtgtaaaacacaatcaagaaatgatgtcatttgttataaaaataaaagc
tgatgtcatgttttgcacatggctcataactaaactcgctttacgggtagaattctacgc
gcgtcgatgtctttgtgatgcgcgcgacatttttgtaggttattgataaaatgaacggat
acgttgcccgacattatcattaaatccttggcgtagaatttgtcgggtccattgtccgtg
tgcgctagcatgcccgtaacggacctcgtacttttggcttcaaaggttttgcgcacagac
aaaatgtgccacacttgcagctctgcatgtgtgcgcgttaccacaaatcccaacggcgca
gtgtacttgttgtatgcaaataaatctcgataaaggcgcggcgcgcgaatgcagctgatc
acgtacgctcctcgtgttccgttcaaggacggtgttatcgacctcagattaatgtttatc
ggccgactgttttcgtatccgctcaccaaacgcgtttttgcattaacattgtatgtcggc
ggatgttctatatctaatttgaataaataaacgataaccgcgttggttttagagggcata
ataaaagaaatattgttatcgtgttcgccattagggcagtataaattgacgttcatgttg
gatattgtttcagttgcaagttgacactggcggcgacaagatcgtgaacaaccaagtgac
ttaattaacaaaatgaacgacgaacttgtcaaacgatctcaatggctcctggagaagctg
cgatacccctgggagatgatgcccctgatgtacgtgatactgaaaggcgccgacggagac
gtcaataaagcgcgccaacggattgacgaaggtatgggggttcttaccggttgggactgt
ttccgaggtatcgatcgggtgtcactcacttcctgggtgctcccattttgtaactgctaa
cgcttattattgagtttcaggacatctgggatcttcggtcgacggagtctattcccaaca
gtgccctggatcaaacactgccatcatgcagtttccgtagcctgttgggctacgctcccc
gacttgacatcccccattctcatcaaacaacaactcaaggcctgagacaacgagtggtgg
aatttgcgcacgaagtcattggtttgtcctggtaaaagttaaaagggttaactggagggt
taattgacacggtttcaactgatggccttattgacacacggatgaaagacttgcacgctt
gaccttctgtctgtactaataaaagttacgttggctgggttttggggtcataatggcccc
aaaatcgaatcgtcataacttcttgaaatacaactcacgtttaagaccattcaagagtat
tagatcatcgtctataatagcagatttgaaatttacttcacatttcggtattgcagtgcc
ccttgcttccacaatggaattagttaaagtttcgagagcattgtcaatatcaagtgttgt
tagcaaacaaatgctaacatcaagattactatcgatgtttgattcacatgtattccaatc
agctcgtaaaacgtcaacatcccaccgcaaaatcgctagtgtttggaggggattttaacc
tccaaattgccaaataacctccaaatcatcacctccaagttagttctaatacactccgtt
atatgaaatatggtggtgcgtcgatcgtcgcaagtttatcgttaaacagtcaataaaatg
agcattttatatcgtgatacatatgagaagatagaggtttcaattaaaacaaatccacat
ggtgtcgctaataaaattgtgcattttaagcgagttatatcctctgatcaagataaaata
gaaaattcgatttttgaatattcaattataagagcctgaataactacaacatgtagtgaa
tcgaaactgatttatgacggtttgtgaaggttacacgtcctaagcatttggattcaagaa
aagcaagagatatgacgaatgtaaactttatcgtatcaatgaagtaactagcgtccagaa
cagtacaaaccaacatcgtaccgtcgtattccactccggtcgttgcaatatctctaggtc
caccgaaaaacactcatgaccaagatcgtgtcgtcgatcttggtccaccgaaacaccgat
gtccatatcgtttcgtcgaacttggaccaacgattcatgcaactgatgacaacgcggccc
ccgggtcgtaccaatatccgaaaaatccaactgttcttctctgcctcgcaggtcaagccg
tggtcaatgaatactcacgattgcacaatctgaacatgttcgacggtgtagagttgcgca
gtacgacgcgccagtccggatgatagactttttacacgatcagcacgacccactgcgctg
cggcaaaggtcgaaccgaaacaagaataaaccacgaagatcagatcgattcgacggaaga
agcaatcgaatgcaaagaagaatcggaacgaagaaaactctaaagcatcgcatatttaca
aagcataacggaaaacccgcaagttcaaactagtgattagtgtaagatgaagcaaagcag
aaatgtagtatctagatttttcaacgttagtttacaaagataaaaaatgaggttggacat
acaatcgtgggtattcgtctgagttcgtcacaactgcaccggaaactgtgaaacagaata
gagccaacctgtgcgcggagaatgttgaggtcattataagcttccttagcatccacgggt
gaaagtcgatcgacggaagcctgcaagactctgtcgatgggctttcgtcctagaagaata
agattaaacctgaaatgtattctcccgtggaatggtttcatttgagtaattctgtatctt
ctccttcccaattccacgaacgcgacgaactctaatacaaacaacataatgaccacagtg
caaatgctgtttaacgataatagcgacatgcagccattctggggctaccacgtggagctc
tacttgggagacagcgttcctaaagagtgtgaaagtgcaaacaagggaggaaaccaagag
tgcaaagcaagtttagagggaaaatttaaaaaatgcaaaacagcagtagtacttaacttt
gaagattgtgtttcgaaagccgaagtgtgttccatctgccaccggaaaaaaacgacgaca
gcagaatcatcaacaagcaacatccatccgaaaaaatccgggaaaccggatottcaacca
accatcctacaatctacaaaccagagattatatctcttcaatcgtttccgacatcggtcg
gtttcggtgcccaaaatgatcggagaaacacttatctctctggagcttgcatgccattgc
gagcgtattttggtagctggccgttgccaaacggctccgcaggtactgctattggaggtt
gtgcacgaccacgttgagtttgccttttgagttggagagtgtgtcttttcgtcatatatt
cggccttttcaagggtgattttcaggctacgtaatgattgtatagtttaaccagctaaaa
catattgatgacaagttctatttcagcaccacaaacaagcctgttaatgtctctcaccgc
aaccattgttctgcgcgcgttataatcagcatagaagtttattttctttgggatgattca
aatattacgtgacgcaaagtttgccaattttagaacccctccctcctccacgtaacggct
tttgtgtgaaaaatttaaattttgtgtatagaccgtagcatttcggaagaccccctccct
tactctgttgagttacgtaaaatttcaacgatccttttgtagttctgaattttatatcag
cgtgcagtgttatgaagatatccacagtataaaatattattttattttaaattctatgct
gattatcaatgtgttactagtggcttttcatactcatgttgcgagctcgatttggcgcac
ggggtcatctacacctgatacctttagggtcgttgggggaccacttagcgtgcacgtacg
gacattcaaaatgttgttcaaatttttttcttaccaagacgagcactttacaatgacaaa
cttataatatttcgcatttttgcgataaagtcaatggcattatgaggcagagggaagcat
catcttccttatgctatccaagtaatttgccattgctttcaagaaatcgaggatttcatc
atgccaaatcagttatcttgtatcaaggcatgtatgtatgttgtttgaagcaactgtata
actgtttgaaactatctaattggtgagctcgtttcatttagtatataataatgataattg
ctatggagacgttatttactagcaagtgatttgacgacctgaaatcggaacaaatagaca
acgtttttataaatacaataaatcagaactttccattattgggtacaaagagttgcgcta
tttcgatactgtcagatcagattttccagcacaacgataccttgatatgcgataacttag
aattagaccttcaaatccatctctccagctatgaacagtcatatagataaagccaatggc
gttatgaggtagcggaaagcgtcatctttccaatgctatctaagtacataatttgctata
gctttctattaatcgtagtttgagagatgcaaagtcagttatctcgtatcaaggtttgat
tgttttggaaattagctaaacagttgacattatcacccgtctttaggggataagcgcata
caaatgtgtatttagttgttcattgaagtaacgtaagataggcaagtatggaaacgagct
caccaaacgtcgaaatacgtctaataaatttgtgttcagcaggatggttcaaaatttatt
tgcatcacctcaaaattacagtacctagtgctgtttgtgacaaacatcaaaaggtaaaat
caaactcgtggcgtcgtgcaatctccatagaatgaacaatttctaaccgtatttgatgga
aagacattgagtatactaacctcttaacagcattacacttttctataaacaataaataat
ttgttctattttacattttctttccccactttcgccccccaataattcaatccctcaaac
aggatacgacatttgttgcatctactttccgaagcgttccagcagacacagacactggcc
ggacgaggagaacatctccgtcacccgcactccgtctgcgtcacggtcgccatgtgccga
ttttcgtacccggtcacagtccagctcgccggatggtgcgctcctccaagaacgtcatca
aggagttcatgcgcttcaaggtgcgcatggagggcaccgtgaacggccacgagttcgaga
tcgagggcgagggcgagggccgcccctacgagggccacaacaccgtgaagctgaaggtga
ccaagggcggccccctgcccttcgcctgggacatcctgtccccccagttccagtacggct
ccaaggtgtacgtgaagcaccccgccgacatccccgactacaagaagctgtccttccccg
agggcttcaagtgggagcgcgtgatgaacttcgaggacggcggcgtggtgaccgtgaccc
aggactcctccctgcaggacggctgcttcatctacaaggtgaagttcatcggcgtgaact
tcccctccgacggccccgtaatgcagaagaagaccatgggctgggaggcctccaccgagc
gcctgtacccccgcgacggcgtgctgaagggcgagatccacaaggccctgaagctgaagg
acggcggccactacctggtggagttcaagtccatctacatggccaagaagcccgtgcagc
tgcccggctactactacgtggactccaagctggacatcacctcccacaacgaggactaca
ccatcgtggagcagtacgagcgcaccgagggccgccaccacctgttcctgtagtaatggt
gagcaagggcgaggagctgttcaccggggtggtgcccatcctggtcgagctggacggcga
cgtaaacggccacaagttcagcgtgtccggcgagggcgagggcgatgccacctacggcaa
gctgaccctgaagttcatctgcaccaccggcaagctgcccgtgccctggcccaccctcgt
gaccaccctgacctacggcgtgcagtgcttcagccgctaccccgaccacatgaagcagca
cgacttcttcaagtccgccatgcccgaaggctacgtccaggagcgcaccatcttcttcaa
ggacgacggcaactacaagacccgcgccgaggtgaagttcgagggcgacaccctggtgaa
ccgcatcgagctgaagggcatcgacttcaaggaggacggcaacatcctggggcacaagct
ggagtacaactacaacagccacaacgtctatatcatggccgacaagcagaagaacggcat
caaggtgaacttcaagatccgccacaacatcgaggacggcagcgtgcagctcgccgacca
ctaccagcagaacacccccatcggcgacggccccgtgctgctgcccgacaaccactacct
gagcacccagtccgccctgagcaaagaccccaacgagaagcgcgatcacatggtcctgct
ggagttcgtgaccgccgccgggatcactctcggcatggacgagctgtacaagtaagcggc
cgcgactctagatcataatcagccataccacatttgtagaggttttacttgctttaaaaa
acctcccacacctccccctgaacctgaaacataaaatgaatgcaattgttgttgttaact
tgtttattgcagcttataatggttacaaataaagcaatagcatcacaaatttcacaaata
aagcatttttttcactgcattctagttgtggtttgtccaaactcatcaatgtatottaaa
gcttgcggccgcaattgatcggctaaatggtatggcaagaaaaggtatgcaatataataa
tcttttattgggtatgcaacgaaaatttgtttcgtcaacgtatgcaatattttttattaa
aagagggtatgcaatgtattttattaaaaacgggtatgcaatataataatcttttattgg
gtatgcaacgaaaatttgtttcgtcaaagtatgcaatattttttattaaaagagggtatg
caatgtattttattaaaaacgggtatgcaataaaaaattatttggtttctctaaaaagta
tgcagcacttattttttgataaggtatgcaacaaaattttactttgccgaaaatatgcaa
tgtttttgcgaataaattcaacgcacacttattacgtgacctgcaggcaaacaaacgcga
gataccggaagtactgaaaaacagtcgctccaggccagtgggaacatcgatgttttgttt
tgacggaccccttactctcgtctcatataaaccgaagccagctaagatggtatacttatt
atcatcttgtgatgaggatgcttctatcaacgaaagttttgtttttttttaataaataaa
taaacataaataaattgtttgttgaatttattattagtatgtaagtgtaaatataataaa
acttaatatctattcaaattaataaataaacctcgatatacagaccgataaaacacatgc
gtcaattttacgcatgattatctttaacgtacgtcacaatatgattatctttctagggtt
aatctactggccgtcgttttacaacgtcgtgactgggaaaaccctggcgttacccaactt
aatcgccttgcagcacatccccctttcgccagctggcgtaatagcgaagaggcccgcacc
gatcgcccttcccaacagttgcgcagcctgaatggcgaatgggacgcgccctgtagcggc
gcattaagcgcggcgggtgtggtggttacgcgcagcgtgaccgctacacttgccagcgcc
ctagcgcccgctcctttcgctttcttcccttcctttctcgccacgttcgccggctttccc
cgtcaagctctaaatcgggggctccctttagggttccgatttagtgctttacggcacctc
gaccccaaaaaacttgattagggtgatggttcacgtagtgggccatcgccctgatagacg
gtttttcgccctttgacgttggagtccacgttctttaatagtggactcttgttccaaact
ggaacaacactcaaccctatctcggtctattcttttgatttataagggattttgccgatt
tcggcctattggttaaaaaatgagctgatttaacaaaaatttaacgcgaattttaacaaa
atattaacgcttacaatttaggtggcacttttcggggaaatgtgcgcggaacccctattt
gtttatttttctaaatacattcaaatatgtatccgctcatgagacaataaccctgataaa
tgcttcaataatattgaaaaaggaagagtatgagtattcaacatttccgtgtcgccctta
ttcccttttttgcggcattttgccttcctgtttttgctcacccagaaacgctggtgaaag
taaaagatgctgaagatcagttgggtgcacgagtgggttacatcgaactggatctcaaca
gcggtaagatccttgagagttttcgccccgaagaacgttttccaatgatgagcactttta
aagttctgctatgtggcgcggtattatcccgtattgacgccgggcaagagcaactcggtc
gccgcatacactattctcagaatgacttggttgagtactcaccagtcacagaaaagcatc
ttacggatggcatgacagtaagagaattatgcagtgctgccataaccatgagtgataaca
ctgcggccaacttacttctgacaacgatcggaggaccgaaggagctaaccgcttttttgc
acaacatgggggatcatgtaactcgccttgatcgttgggaaccggagctgaatgaagcca
taccaaacgacgagcgtgacaccacgatgcctgtagcaatggcaacaacgttgcgcaaac
tattaactggcgaactacttactctagcttcccggcaacaattaatagactggatggagg
cggataaagttgcaggaccacttctgcgctcggcccttccggctggctggtttattgctg
ataaatctggagccggtgagcgtgggtctcgcggtatcattgcagcactggggccagatg
gtaagccctcccgtatcgtagttatctacacgacggggagtcaggcaactatggatgaac
gaaatagacagatcgctgagataggtgcctcactgattaagcattggtaactgtcagacc
aagtttactcatatatactttagattgatttaaaacttcatttttaatttaaaaggatct
aggtgaagatcctttttgataatctcatgaccaaaatcccttaacgtgagttttcgttcc
actgagcgtcagaccccgtagaaaagatcaaaggatcttc
Plasmid sequence of vector 1174I
SEQ ID NO: 2
ttgagatcctttttttctgcgcgtaatctgctgcttgcaaacaaaaaaaccaccgctacc
agcggtggtttgtttgccggatcaagagctaccaactctttttccgaaggtaactggctt
cagcagagcgcagataccaaatactgttcttctagtgtagccgtagttaggccaccactt
caagaactctgtagcaccgcctacatacctcgctctgctaatcctgttaccagtggctgc
tgccagtggcgataagtcgtgtcttaccgggttggactcaagacgatagttaccggataa
ggcgcagcggtcgggctgaacggggggttcgtgcacacagcccagcttggagcgaacgac
ctacaccgaactgagatacctacagcgtgagctatgagaaagcgccacgcttcccgaagg
gagaaaggcggacaggtatccggtaagcggcagggtcggaacaggagagcgcacgaggga
gcttccagggggaaacgcctggtatctttatagtcctgtcgggtttcgccacctctgact
tgagcgtcgatttttgtgatgctcgtcaggggggcggagcctatggaaaaacgccagcaa
cgcggcctttttacggttcctggccttttgctggccttttgctcacatgttctttcctgc
gttatcccctgattctgtggataaccgtattaccgcctttgagtgagctgataccgctcg
ccgcagccgaacgaccgagcgcagcgagtcagtgagcgaggaagcggaagagcgcccaat
acgcaaaccgcctctccccgcgcgttggccgattcattaatgcagctggcacgacaggtt
tcccgactggaaagcgggcagtgagcgcaacgcaattaatgtgagttagctcactcatta
ggcaccccaggctttacactttatgcttccggctcgtatgttgtgtggaattgtgagcgg
ataacaatttcacacaggaaacagctatgaccatgattacgccaagctcgaaattaaccc
tcactaaagggattaaccctagaaagatagtctgcgtaaaattgacgcatgcattcttga
aatattgctctctctttctaaatagcgcgaatccgtcgctgtgcatttaggacatctcag
tcgccgcttggagctcccgtgaggcgtgcttgtcaatgcggtaagtgtcactgattttga
actataacgaccgcgtgagtcaaaatgacgcatgattatcttttacgtgacttttaagat
ttaactcatacgataattatattgttatttcatgttctacttacgtgataacttattata
tatatattttcttgttatagatatctgtcgacatgcccgccgtgaccgtcgagaacccgc
tgacgctgccccgcgtatccgcacccgccgacgccgtcgcacgtcccgtgctcaccgtga
ccaccgcgcccagcggtttcgagggcgagggcttcccggtgcgccgcgcgttcgccggga
tcaactaccgccacctcgacccgttcatcatgatggaccagatgggtgaggtggagtacg
cgcccggggagcccaagggcacgccctggcacccgcaccgcggcttcgagaccgtgacct
acatcgtcgacgggcccgcgtgcaaaagcgtgtttttttgcagtgcaaaaaagttggtgg
tggggaggccaccgagtatgccgcggaattgatcggctaaatggtatggcaagaaaaggt
atgcaatataataatcttttattgggtatgcaacgaaaatttgtttcgtcaacgtatgca
atattttttattaaaagagggtatgcaatgtattttattaaaaacgggtatgcaatataa
taatcttttattgggtatgcaacgaaaatttgtttcgtcaaagtatgcaatattttttat
taaaagagggtatgcaatgtattttattaaaaacgggtatgcaataaaaaattatttggt
ttctctaaaaagtatgcagcacttattttttgataaggtatgcaacaaaattttactttg
ccgaaaatatgcaatgtttttgcgaataaattcaacgcacacttattacgtgaggtaccg
cgtaaaacacaatcaagtatgagtcataagctgatgtcatgttttgcacacggctcataa
ccgaactggctttacgagtagaattctacttgtaacgcacgatcagtggatgatgtcatt
tgtttttcaaatcgagatgatgtcatgttttgcacacggctcataaactcgctttacgag
tagaattctacgtgtaacgcacgatcgattgatgagtcatttgttttgcaatatgatatc
atacaatatgactcatttgtttttcaaaaccgaacttgatttacgggtagaattctactt
gtaaagcacaatcaaaaagatgatgtcatttgtttttcaaaactgaactcgctttacgag
tagaattctacgtgtaaaacacaatcaagaaatgatgtcatttgttataaaaataaaagc
tgatgtcatgttttgcacatggctcataactaaactcgctttacgggtagaattctacgc
gcgtcgatgtctttgtgatgcgcgcgacatttttgtaggttattgataaaatgaacggat
acgttgcccgacattatcattaaatccttggcgtagaatttgtcgggtccattgtccgtg
tgcgctagcatgcccgtaacggacctcgtacttttggcttcaaaggttttgcgcacagac
aaaatgtgccacacttgcagctctgcatgtgtgcgcgttaccacaaatcccaacggcgca
gtgtacttgttgtatgcaaataaatctcgataaaggcgcggcgcgcgaatgcagctgatc
acgtacgctcctcgtgttccgttcaaggacggtgttatcgacctcagattaatgtttatc
ggccgactgttttcgtatccgctcaccaaacgcgtttttgcattaacattgtatgtcggc
ggatgttctatatctaatttgaataaataaacgataaccgcgttggttttagagggcata
ataaaagaaatattgttatcgtgttcgccattagggcagtataaattgacgttcatgttg
gatattgtttcagttgcaagttgacactggcggcgacaagatcgtgaacaaccaagtgac
ttaattaacaaaatgaacgacgaacttgtcaaacgatctcaatggctcctggagaagctg
cgatacccctgggagatgatgcccctgatgtacgtgatactgaaaggcgccgacggagac
gtcaataaagcgcgccaacggattgacgaaggtatgggggttcttaccggttgggactgt
ttccgaggtatcgatcgggtgtcactcacttcctgggtgctcccattttgtaactgctaa
cgcttattattgagtttcaggacatctgggatcttcggtcgacggagtctattcccaaca
gtgccctggatcaaacactgccatcatgcagtttccgtagcctgttgggctacgctcccc
gacttgacatcccccattctcatcaaacaacaactcaaggcctgagacaacgagtggtgg
aatttgcgcacgaagtcattggtttgtcctggtaaaagttaaaagggttaactggagggt
taattgacacggtttcaactgatggccttattgacacacggatgaaagacttgcacgctt
gaccttctgtctgtactaataaaagttacgttggctgggttttggggtcataatggcccc
aaaatcgaatcgtcataacttcttgaaatacaactcacgtttaagaccattcaagagtat
tagatcatcgtctataatagcagatttgaaatttacttcacatttcggtattgcagtgcc
ccttgcttccacaatggaattagttaaagtttcgagagcattgtcaatatcaagtgttgt
tagcaaacaaatgctaacatcaagattactatcgatgtttgattcacatgtattccaatc
agctcgtaaaacgtcaacatcccaccgcaaaatcgctagtgtttggaggggattttaacc
tccaaattgccaaataacctccaaatcatcacctccaagttagttctaatacactccgtt
atatgaaatatggtggtgcgtcgatcgtcgcaagtttatcgttaaacagtcaataaaatg
agcattttatatcgtgatacatatgagaagatagaggtttcaattaaaacaaatccacat
ggtgtcgctaataaaattgtgcattttaagcgagttatatcctctgatcaagataaaata
gaaaattcgatttttgaatattcaattataagagcctgaataactacaacatgtagtgaa
tcgaaactgatttatgacggtttgtgaaggttacacgtcctaagcatttggattcaagaa
aagcaagagatatgacgaatgtaaactttatcgtatcaatgaagtaactagcgtccagaa
cagtacaaaccaacatcgtaccgtcgtattccactccggtcgttgcaatatctctaggtc
caccgaaaaacactcatgaccaagatcgtgtcgtcgatcttggtccaccgaaacaccgat
gtccatatcgtttcgtcgaacttggaccaacgattcatgcaactgatgacaacgcggccc
ccgggtcgtaccaatatccgaaaaatccaactgttcttctctgcctcgcaggtcaagccg
tggtcaatgaatactcacgattgcacaatctgaacatgttcgacggtgtagagttgcgca
gtacgacgcgccagtccggatgatagactttttacacgatcagcacgacccactgcgctg
cggcaaaggtcgaaccgaaacaagaataaaccacgaagatcagatcgattcgacggaaga
agcaatcgaatgcaaagaagaatcggaacgaagaaaactctaaagcatcgcatatttaca
aagcataacggaaaacccgcaagttcaaactagtgattagtgtaagatgaagcaaagcag
aaatgtagtatctagatttttcaacgttagtttacaaagataaaaaatgaggttggacat
acaatcgtgggtattcgtctgagttcgtcacaactgcaccggaaactgtgaaacagaata
gagccaacctgtgcgcggagaatgttgaggtcattataagcttccttagcatccacgggt
gaaagtcgatcgacggaagcctgcaagactctgtcgatgggctttcgtcctagaagaata
agattaaacctgaaatgtattctcccgtggaatggtttcatttgagtaattctgtatctt
ctccttcccaattccacgaacgcgacgaactctaatacaaacaacataatgaccacagtg
caaatgctgtttaacgataatagcgacatgcagccattctggggctaccacgtggagctc
tacttgggagacagcgttcctaaagagtgtgaaagtgcaaacaagggaggaaaccaagag
tgcaaagcaagtttagagggaaaatttaaaaaatgcaaaacagcagtagtacttaacttt
gaagattgtgtttcgaaagccgaagtgtgttccatctgccaccggaaaaaaacgacgaca
gcagaatcatcaacaagcaacatccatccgaaaaaatccgggaaaccggatcttcaacca
accatcctacaatctacaaaccagagattatatctcttcaatcgtttccgacatcggtcg
gtttcggtgcccaaaatgatcggagaaacacttatctctctggagcttgcatgccattgc
gagcgtattttggtagctggccgttgccaaacggctccgcaggtactgctattggaggtt
gtgcacgaccacgttgagtttgccttttgagttggagagtgtgtcttttcgtcatatatt
cggccttttcaagggtgattttcaggctacgtaatgattgtatagtttaaccagctaaaa
catattgatgacaagttctatttcagcaccacaaacaagcctgttaatgtctctcaccgc
aaccattgttctgcgcgcgttataatcagcatagaagtttattttctttgggatgattca
aatattacgtgacgcaaagtttgccaattttagaacccctccctcctccacgtaacggct
tttgtgtgaaaaatttaaattttgtgtatagaccgtagcatttcggaagaccccctccct
tactctgttgagttacgtaaaatttcaacgatccttttgtagttctgaattttatatcag
cgtgcagtgttatgaagatatccacagtataaaatattattttattttaaattctatgct
gattatcaatgtgttactagtggcttttcatactcatgttgcgagctcgatttggcgcac
ggggtcatctacacctgatacctttagggtcgttgggggaccacttagcgtgcacgtacg
gacattcaaaatgttgttcaaatttttttcttaccaagacgagcactttacaatgacaaa
cttataatatttcgcatttttgcgataaagtcaatggcattatgaggcagagggaagcat
catcttccttatgctatccaagtaatttgccattgctttcaagaaatcgaggatttcatc
atgccaaatcagttatcttgtatcaaggcatgtatgtatgttgtttgaagcaactgtata
actgtttgaaactatctaattggtgagctcgtttcatttagtatataataatgataattg
ctatggagacgttatttactagcaagtgatttgacgacctgaaatcggaacaaatagaca
acgtttttataaatacaataaatcagaactttccattattgggtacaaagagttgcgcta
tttcgatactgtcagatcagattttccagcacaacgataccttgatatgcgataacttag
aattagaccttcaaatccatctctccagctatgaacagtcatatagataaagccaatggc
gttatgaggtagcggaaagcgtcatctttccaatgctatctaagtacataatttgctata
gctttctattaatcgtagtttgagagatgcaaagtcagttatctcgtatcaaggtttgat
tgttttggaaattagctaaacagttgacattatcacccgtctttaggggataagcgcata
caaatgtgtatttagttgttcattgaagtaacgtaagataggcaagtatggaaacgagct
caccaaacgtcgaaatacgtctaataaatttgtgttcagcaggatggttcaaaatttatt
tgcatcacctcaaaattacagtacctagtgctgtttgtgacaaacatcaaaaggtaaaat
caaactcgtggcgtcgtgcaatctccatagaatgaacaatttctaaccgtatttgatgga
aagacattgagtatactaacctcttaacagcattacacttttctataaacaataaataat
ttgttctattttacattttctttccccactttcgccccccaataattcaatccctcaaac
aggatacgacatttgttgcatctactttccgaagcgttccagcagacacagacactggcc
ggacgaggagaacatctccgtcacccgcactccgtctgcgtcacggtcgccatgtgccga
ttttcgtacccggtcacagtccagctcgccggatggtgcgctcctccaagaacgtcatca
aggagttcatgcgcttcaaggtgcgcatggagggcaccgtgaacggccacgagttcgaga
tcgagggcgagggcgagggccgcccctacgagggccacaacaccgtgaagctgaaggtga
ccaagggcggccccctgcccttcgcctgggacatcctgtccccccagttccagtacggct
ccaaggtgtacgtgaagcaccccgccgacatccccgactacaagaagctgtccttccccg
agggcttcaagtgggagcgcgtgatgaacttcgaggacggcggcgtggtgaccgtgaccc
aggactcctccctgcaggacggctgcttcatctacaaggtgaagttcatcggcgtgaact
tcccctccgacggccccgtaatgcagaagaagaccatgggctgggaggcctccaccgagc
gcctgtacccccgcgacggcgtgctgaagggcgagatccacaaggccctgaagctgaagg
acggcggccactacctggtggagttcaagtccatctacatggccaagaagcccgtgcagc
tgcccggctactactacgtggactccaagctggacatcacctcccacaacgaggactaca
ccatcgtggagcagtacgagcgcaccgagggccgccaccacctgttcctgtagtaatggt
gagcaagggcgaggagctgttcaccggggtggtgcccatcctggtcgagctggacggcga
cgtaaacggccacaagttcagcgtgtccggcgagggcgagggcgatgccacctacggcaa
gctgaccctgaagttcatctgcaccaccggcaagctgcccgtgccctggcccaccctcgt
gaccaccctgacctacggcgtgcagtgcttcagccgctaccccgaccacatgaagcagca
cgacttcttcaagtccgccatgcccgaaggctacgtccaggagcgcaccatcttcttcaa
ggacgacggcaactacaagacccgcgccgaggtgaagttcgagggcgacaccctggtgaa
ccgcatcgagctgaagggcatcgacttcaaggaggacggcaacatcctggggcacaagct
ggagtacaactacaacagccacaacgtctatatcatggccgacaagcagaagaacggcat
caaggtgaacttcaagatccgccacaacatcgaggacggcagcgtgcagctcgccgacca
ctaccagcagaacacccccatcggcgacggccccgtgctgctgcccgacaaccactacct
gagcacccagtccgccctgagcaaagaccccaacgagaagcgcgatcacatggtcctgct
ggagttcgtgaccgccgccgggatcactctcggcatggacgagctgtacaagtaagcggc
cgcgactctagatcataatcagccataccacatttgtagaggttttacttgctttaaaaa
acctcccacacctccccctgaacctgaaacataaaatgaatgcaattgttgttgttaact
tgtttattgcagcttataatggttacaaataaagcaatagcatcacaaatttcacaaata
aagcatttttttcactgcattctagttgtggtttgtccaaactcatcaatgtatcttaaa
gcttgcggccgcaattgatcggctaaatggtatggcaagaaaaggtatgcaatataataa
tcttttattgggtatgcaacgaaaatttgtttcgtcaacgtatgcaatattttttattaa
aagagggtatgcaatgtattttattaaaaacgggtatgcaatataataatcttttattgg
gtatgcaacgaaaatttgtttcgtcaaagtatgcaatattttttattaaaagagggtatg
caatgtattttattaaaaacgggtatgcaataaaaaattatttggtttctctaaaaagta
tgcagcacttattttttgataaggtatgcaacaaaattttactttgccgaaaatatgcaa
tgtttttgcgaataaattcaacgcacacttattacgtgacctgcaggcaaacaaacgcga
gataccggaagtactgaaaaacagtcgctccaggccagtgggaacatcgatgttttgttt
tgacggaccccttactctcgtctcatataaaccgaagccagctaagatggtatacttatt
atcatcttgtgatgaggatgcttctatcaacgaaagttttgtttttttttaataaataaa
taaacataaataaattgtttgttgaatttattattagtatgtaagtgtaaatataataaa
acttaatatctattcaaattaataaataaacctcgatatacagaccgataaaacacatgc
gtcaattttacgcatgattatctttaacgtacgtcacaatatgattatctttctagggtt
aatctactggccgtcgttttacaacgtcgtgactgggaaaaccctggcgttacccaactt
aatcgccttgcagcacatccccctttcgccagctggcgtaatagcgaagaggcccgcacc
gatcgcccttcccaacagttgcgcagcctgaatggcgaatgggacgcgccctgtagcggc
gcattaagcgcggcgggtgtggtggttacgcgcagcgtgaccgctacacttgccagcgcc
ctagcgcccgctcctttcgctttcttcccttcctttctcgccacgttcgccggctttccc
cgtcaagctctaaatcgggggctccctttagggttccgatttagtgctttacggcacctc
gaccccaaaaaacttgattagggtgatggttcacgtagtgggccatcgccctgatagacg
gtttttcgccctttgacgttggagtccacgttctttaatagtggactcttgttccaaact
ggaacaacactcaaccctatctcggtctattcttttgatttataagggattttgccgatt
tcggcctattggttaaaaaatgagctgatttaacaaaaatttaacgcgaattttaacaaa
atattaacgcttacaatttaggtggcacttttcggggaaatgtgcgcggaacccctattt
gtttatttttctaaatacattcaaatatgtatccgctcatgagacaataaccctgataaa
tgcttcaataatattgaaaaaggaagagtatgagtattcaacatttccgtgtcgccctta
ttcccttttttgcggcattttgccttcctgtttttgctcacccagaaacgctggtgaaag
taaaagatgctgaagatcagttgggtgcacgagtgggttacatcgaactggatctcaaca
gcggtaagatccttgagagttttcgccccgaagaacgttttccaatgatgagcactttta
aagttctgctatgtggcgcggtattatcccgtattgacgccgggcaagagcaactcggtc
gccgcatacactattctcagaatgacttggttgagtactcaccagtcacagaaaagcatc
ttacggatggcatgacagtaagagaattatgcagtgctgccataaccatgagtgataaca
ctgcggccaacttacttctgacaacgatcggaggaccgaaggagctaaccgcttttttgc
acaacatgggggatcatgtaactcgccttgatcgttgggaaccggagctgaatgaagcca
taccaaacgacgagcgtgacaccacgatgcctgtagcaatggcaacaacgttgcgcaaac
tattaactggcgaactacttactctagcttcccggcaacaattaatagactggatggagg
cggataaagttgcaggaccacttctgcgctcggcccttccggctggctggtttattgctg
ataaatctggagccggtgagcgtgggtctcgcggtatcattgcagcactggggccagatg
gtaagccctcccgtatcgtagttatctacacgacggggagtcaggcaactatggatgaac
gaaatagacagatcgctgagataggtgcctcactgattaagcattggtaactgtcagacc
aagtttactcatatatactttagattgatttaaaacttcatttttaatttaaaaggatct
aggtgaagatcctttttgataatctcatgaccaaaatcccttaacgtgagttttcgttcc
actgagcgtcagaccccgtagaaaagatcaaaggatcttc

### EXAMPLES

The disclosure is further described in the following examples, which do not limit the scope of the disclosure.

Aedes aegypti

### Molecular Cloning and Transgenesis

To create the endogenous AaeDsx splicing module construct, the fragment of endogenous exons and introns from the genomic DNA of Ae. aegypti was amplified using PCR. Then, the previous mCherry and EGFP containing plasmid, 1122I, was linearized using the restriction enzyme PacI. The linearized 1122I plasmid and the fragment of endogenous exons and introns were used in a Gibson enzymatic assembly method to build the 1174CX plasmid. To open the reading frame of the female-specific product and allow for in-frame expression of the mCherry coding sequence, the endogenous exon 5b was substituted with an engineered exon 5b that had the stop codons removed. The sequence of the engineered exon 5b was synthesized using the gBlocks® Gene Fragment service. The endogenous exon 5b was removed by cutting it with the restriction enzymes PmlI and SnaBI, and then Gibson assembly was used to incorporate the engineered exon 5b containing fragment into the cut 1174CX plasmid, resulting in the 1174C plasmid.

To generate a plasmid for sex-specific expression of DsRed and EGFP, the fragments of the engineered exon 5b and DsRed coding sequences from the 1174C plasmid and the previous 874Y plasmid were amplified. Then, these two fragments were fused together using overlapping PCR. Finally, omega PCR was used to substitute the sequence from the mCherry coding sequence to the 3×P3 promoter in the 1174C plasmid with the DsRed coding sequence, resulting in the 1174D plasmid. During each cloning step, single colonies were selected and cultured in LB medium with ampicillin. Then, the plasmids (using the Zymo Research Zyppy plasmid miniprep kit) were extracted and subsequently underwent Sanger sequencing. The final plasmids were maxi-prepped (using the Zymo Research ZvmoPURE II Plasmid Maxiprep kit) and fully sequenced by Primordium. All primers are listed in Table 1. The complete annotated plasmid sequences and plasmid DNA are available at Addgene (ID: 200012).

Transgenic lines were created by microinjecting preblastoderm stage embryos with a mixture of the piggyBac plasmid and a transposase helper plasmid. Four days after microinjection, the G0 embryos were hatched and the surviving pupae were separated and sexed. The pupae were placed in separate cages for males and females, along with wild-type male pupae in the female cages and wild-type female pupae in the male cages, in a 5:1 ratio. After several days to allow for development and mating, a blood meal was provided and eggs were collected, aged, and hatched. The larvae with positive fluorescent markers were isolated using a fluorescent stereomicroscope. To isolate separate insertion events, male transformants with fluorescent markers were crossed with female transformants without fluorescent markers, and separate lines were established. The individual genetic sexing lines (1174D) were maintained as mixtures of homozygotes and heterozygotes, with periodic elimination of wild-type individuals. The genetic sexing line (1174D) was homozygosed through approximately ten generations of single-pair sibling matings, selecting individuals with the brightest marker expression each generation.

### Mosquito Rearing and Maintenance

Ae. aegypti mosquitoes were obtained from the Liverpool strain, which was previously used to generate the reference genome. These mosquitoes were raised in incubators at 30° C. with 20-40% humidity and a 12-hour light/dark cycle in cages (Bugdorm. 24.5×24.5×24.5 cm). Adults were given 10% (m/V) aqueous sucrose ad libitum, and females were given a blood meal by feeding on anesthetized mice for approximately 15 minutes. Oviposition substrates were provided about 3 days after the blood meal. Eggs were collected, aged for about 4 days to allow for embryonic development, and then hatched in deionized water in a vacuum chamber. Approximately 400 larvae were reared in plastic containers (Sterilite, 34.6×21×12.4 cm, USA) with about 3 liters of deionized water and fed fish food (TetraMin Tropical Flakes, Tetra Werke. Melle, Germany). For genetic crosses, female virginity was ensured by separating and sexing the pupae under the microscope based on sex-specific morphological differences in the genital lobe shape (at the end of the pupal abdominal segments just below the paddles) before releasing them to eclose in cages. These general rearing procedures were followed unless otherwise noted. In order to increase the number of homozygotes in the 1174D transgenic line, both the high-intensity GFP pupae and female GFP-negative pupae were transferred to a cage and allowed to mate after eclosion. Female mosquitoes were fed a blood meal, and five adult females were individually transferred to egg tubes for colonization and egg collection. The eggs from each colony were hatched and reared. The colonies with a higher proportion of female EGFP negatives and male EGFP positives were selected for colonization in the next generation. After a few rounds of colonization, the colonies with 100% of males in the strong EGFP positive group and 100% of females in the GFP negative group were selected and propagated for expansion.

TABLE 1

| Sequences of primers and gBlock fragment used in this study | | | | |
|---|---|---|---|---|
| Name | Sequence | Amplicon | Usage | Note/SEQ ID NO |
| Hr5Ie1- | ATCGTGAACAACCAAGTGACTTAATTAACAAAA | Hr5Ie1 | AaeDsx | SEQ ID NO: 3 |
| Exon4F | TGAACGACGAACTTGTCAAACGATCTC | to 5′ of | splicing | |
| Intron4- | GCGATTTTGCGGTGGGATGTTGACGTTTTACGAG | Intron4 | module | SEQ ID NO: 4 |
| 1R | CTGATTGGAATACATG | | assembly | |
| Intron4- | CATGTATTCCAATCAGCTCGTAAAACGTCAACAT | 3′ of | | SEQ ID NO: 5 |
| 2F | CCCACCGCAAAATCGC | Intron4 | | |
| Intron6- | CTCATAATGCCATTGACTTTATCGCAAAAATGCG | to 5′ of | | SEQ ID NO: 6 |
| 1R | AAATATTATAAGTTTG | Intron6 | | |
| Intron6- | CAAACTTATAATATTTCGCATTTTTGCGATAAAG | 3′ of | | SEQ ID NO: 7 |
| 2F | TCAATGGCATTATGAG | Intron6 | | |
| Exon6R | GTTATCCTCCTCGCCCTTGCTCACCATTCCGGCG | to Exon6 | | SEQ ID NO: 8 |
| | AGCTGGACTGTGACCGGGTAC | | | |
| Engineered | tgcaaatgctgtttaacgataatagcgacatgcagccattctggggc | | To remove | 1. The initial |
| Exon5b | taccacgtgGAGctctacttgGGAgacagcgttcctaaagagtgtga | | nine stop | nucleotides of nine |
| | aagtgcaaacaagGGAGGAaaccaaGAGtgcaaagcaagtttagagg | | codons | stop codons in |
| | gaaaatttaaaaaatgcaaaacagcagtagtacttaactttGAAgat | | located in | exon5b were |
| | tgtgtttcgaaagccgaagtgtgttccatctgccaccggaaaaaaac | | endogenous | marked in red due |
| | gacgacagcagaatcatcaacaagcaacatccatccgaaaaaatccg | | exon5b | to a T to G |
| | ggaaaccggatcttcaaccaaccatcctacaatctacaaaccagaga | | | substitution. |
| | ttatatctcttcaatcgtttccgacatcggtcggtttcggtgcccaa | | | 2. A single |
| | aatgatcGGAGAAacacttatctctctgGAGcttgcatgccattgcg | | | nucleotide deletion |
| | agcgtattttggtagctggccgttgccaaacggctccGCaggtactg | | | (GAC to GC) |
| | ctattggaggttgtgcacgaccacgttgagtttgccttttgagttgg | | | |
| | agagtgtgtcttttcgtcatatattcggccttttcaagggtgatttt | | | resulting in an |
| | caggctacgtaatgattgtatagtttaaccagctaaaacatattgat | | | in-frame male |
| | gacaagttctatttcagcaccacaaacaagcctgttaatgtctctca | | | splicing product |
| | ccgcaaccattgttctgcgcgcgttataatcagcatagaagtttatt | | | was indicated with |
| | ttctttgggatgattcaaatattacgtgacgcaaagtttgccaattt | | | green highlighting. |
| | tagaacccctccctcctccacgtaacggcttttgtgtgaaaaattta | | | SEQ ID NO: 9 |
| | aattttgtgtatagaccgtagcatttcggaagaccccctcccttact | | | |
| | ctgttgagttacgtaaaatttcaacgatccttttgtagttctgaatt | | | |
| | ttatatcag | | | |
| 1174D | GTTGCCAAACGGCTCCGCAGGTACTGCTATTGGA | Engi- | Engineered | SEQ ID NO: 10 |
| Exon5bF | GGTTG | neered | Exon5b, | |
| | | Exon5b to | DsRed, eGFP | |
| 1174D | GTTCTTGGAGGAGCGCACCATCCGGCGAGCTGG | DsRed | fragments | SEQ ID NO: 11 |
| Exon6R | ACTGTGAC | | assembly | |
| 644A | ATGGTGCGCTCCTCCAAGAAC | DsRed to | | SEQ ID NO: 12 |
| 1174D | GAACAGCTCCTCGCCCTTGCTCACCATTACTACA | eGFP | | SEQ ID NO: 13 |
| DsRedR | GGAACAGGTGGTGGCGGCCCTC | | | |

### COPAS® Fluorescent Sorting, Sexing and Imaging

To determine the precise number of larvae in COPAS® clusters, the COPAS® raw data was filtered based on the optical density and size measurements of the individuals, “log(EXT)” and “log(TOF),” to remove outliers such as egg debris and dust. Then, a filter was applied based on the individuals' fluorescence measurements, “log(first fluorescence)” and “log(second fluorescence)”, to further refine the data. Finally, the fluorescence measurements were automatically clustered and denoised using Density-Based Spatial Clustering of Applications with Noise (DBSCAN). COPAS® sorting was performed largely as described for Anopheles larvae. Aedes eggs stuck to their egg laying paper were briefly rinsed to eliminate dust and debris, immersed in deionized water in a small container, and their hatching was stimulated under partial vacuum (25% of atmospheric pressure) in a vacuum chamber for 30-60 minutes. They were then incubated overnight at 28° C. to maximize larval hatching. On the next day, resulting unfed neonate larvae were transferred to the reservoir of a large particle flow cytometry COPAS® SELECT instrument (Union Biometrica, Holliston, MA, USA) equipped with a multiline argon laser (488, 514 nm) and a diode laser (670 nm). Larvae were analyzed and sorted with the Biosort5281 software using a 488 nm filter and the following acquisition parameters: Green PMT 500, Red PMT 600, Delay 8; Width 6, pure mode with superdrops. Flow rate was kept between 20 and 70 objects per second through adjusting of the concentration of larvae in the sample. Larvae identified as males (GFP positive) were dispensed in a Petri dish. In these conditions, sorting speed ranged from 4000 to 7400 larvae in 10 minutes (+6 minutes for system initialization and 6 minutes for system cleaning and shutdown), the total number of sorted larvae being limited by the number of available larvae. Sorted larval counts provided by the COPAS® software were recorded on sorting. For quality control of sorted larvae, the reservoir and fluidics of the instrument were carefully rinsed and the sorted larvae analyzed by passing them once more in the machine. In some experiments, objects falling outside the GFP positive gate were collected in “Enrich” mode to remove GFP negative contaminants from the pool of GFP positive larvae, and verified by microscopy. Mosquitoes were examined, scored, and imaged using the Leica M165FC fluorescent stereomicroscope equipped with the Leica DMC2900 camera. For higher-resolution images, a Leica DM4B upright microscope equipped with a VIEW4K camera was used. To distinguish between male and female pupae in mosquitoes, a microscope was used to observe the sex-specific morphological differences in the genital lobe shape located at the end of the pupal abdominal segments just below the paddles. This approach ensured the inclusion of both male and female pupae in the present experimental selection.

### Determination of Genome Integration Sites

To determine the transgene insertion sites. Oxford Nanopore genome DNA sequencing was performed. Genomic DNA was extracted using the Blood & Cell Culture DNA Midi Kit (Qiagen, Cat. No./ID: 13343) from 5 adult males and 5 adult females of SEPARATOR, following the manufacturer's protocol. The sequencing library was prepared using the Oxford Nanopore SQK-LSK110 genomic library kit and sequenced on a single MinION flowcell (R9.4.1) for 72 hrs. Basecalling was performed with ONT Guppy base calling software version 6.4.6 using dna_r9.4.1_450bps_sup model generating 3.03 million reads above the quality threshold of Q≥10 with N50 of 7941 bp and total yield of 11.08 Gb. To identify transgene insertion sites, nanopore reads were mapped to plasmids carrying SEPARATOR (1174D, Addgene as plasmid #200012) using minimap2 and further aligned them to the AaegL5.0 genome (GCF_002204515.2). Subsequently, the average depth of coverage was calculated for the three autosomes and the transgene using samtools and the results was visualized in R. The coverage depths for chr1, chr2, and chr3 were determined to be 6.31, 6.30, and 6.08, respectively. Interestingly, the coverage depth for SEPARATOR transgene was notably higher at 16.14. Based on the coverage analysis, it appears that the SEPARATOR transgene is present in three copies (FIG. 7). By examining the read alignments using Interactive Genomics Viewer (IGV), the exact insertion sites were determined. Notably, three copies of the SEPARATOR construct sequence were identified. The three integration sites are NC_035109.1:92046983. NC_035108.1:444508475 and NC_035107.1:299022928. This finding aligns with the results of the depth of coverage analysis, further supporting the presence of three insertion sites. The second integration site on NC_035108.1 overlaps with the AAEL005024 gene, which is currently classified as an uncharacterized protein. However, the other two integration sites do not overlap with any known genes. The nanopore sequencing data has been deposited to the NCBI SRA (PRJNA985064).

### RNA Sequencing (RNA-Seq) Analysis

To quantify target gene reduction and expression from transgenes as well as to assess global expression patterns, Illumina RNA sequencing was performed. Total RNA was extracted using miRNeasy Tissue/Cells Advanced Mini Kit (Qiagen, Cat. No./ID: 217604) from 50 GFP-positive male mosquitoes and 50 GFP-negative female mosquitoes at L1 larva stage in biological triplicate (6 samples total), following the manufacturer's protocol. Genomic DNA was depleted using the gDNA eliminator column provided by the kit. RNA integrity was assessed using the RNA 6000 Pico Kit for Bioanalyzer (Agilent Technologies, Cat. No./ID: #5067-1513), and mRNA was isolated from 1 g of total RNA using NEBNext Poly(A) mRNA Magnetic Isolation Module (NEB, Cat. No/ID: E7490). RNA-seq libraries were constructed using the NEBNext Ultra II RNA Library Prep Kit for Illumina (NEB, Cat. No/ID: E7770) following the manufacturer's protocols. Briefly, mRNA was fragmented to an average size of 200 nt by incubating at 94° C. for 15 min in the first strand buffer. cDNA was then synthesized using random primers and ProtoScript II Reverse Transcriptase followed by second strand synthesis using NEB Second Strand Synthesis Enzyme Mix. Resulting DNA fragments were end-repaired, dA tailed, and ligated to NEBNext hairpin adaptors (NEB, Cat. No./ID: E7335). Following ligation, adaptors were converted to the “Y” shape by treating with USER enzyme, and DNA fragments were size selected using Agencourt AMPure XP beads (Beckman Coulter #A63880) to generate fragment sizes between 250-350 bp. Adaptor-ligated DNA was PCR amplified followed by AMPure XP bead clean up. Libraries were quantified using a Qubit dsDNA HS Kit (ThermoFisher Scientific, Cat. No/ID: Q32854), and the size distribution was confirmed using a High Sensitivity DNA Kit for Bioanalyzer (Agilent Technologies, Cat. No/ID: 5067-4626). Libraries were sequenced on an Illumina NextSeq2000 in paired end mode with the read length of 50 nt and sequencing depth of 25 million reads per library. Base calls and FASTQ generation were performed with DRAGEN 3.8.4. The reads were mapped to the AaegL5.0 genome (GCF_002204515.2) supplemented with SEPARATOR transgene sequences using STAR. On average, ˜97.4% of the reads were mapped. The analysis of RNA-Seq data was performed using an integrated web application called iDEP75. TPM values were calculated from counts produced by feature counts and combined. Hierarchical clustering of the data shows that for each genotype, all replicates cluster together, as expected (FIGS. 9A-9B). DESeq2 was then used to perform differential expression analyses between male (GFP-positive) and female (GFP-negative) at L1 larvae stage (FIG. 9C). For each DESeq2 comparison, gene ontology enrichments were performed on significantly differentially expressed genes. (FIG. 9D-9E). Illumina RNA sequencing data has been deposited to the NCBI-SRA (PRJNA985064). For transcriptome comparing analysis, 47 files consisting of six developmental stages (L3 larvae, L4 larvae, early pupae, mid pupae, late pupae, and adult carcass) were acquired from SRA. These files were then aligned to the AaegL5 genome (GCF_002204515.2) using STAR.

### Example 1—Generation of Sex Sorting Gene Expression System

To generate a sex-specific gene expression system in Ae. aegypti, a transformation vector was constructed with piggyBac inverted terminal repeats which flanked the effector region. The effector region is allowed for genome integration via piggyBac inverted terminal repeats. The effector region containing the promoter region, sex-specific splicing element, reporter genes, and transcription terminator was constructed for sex-specific gene expression. Ae. aegypti doublesex (Aadsx) was chosen as a candidate gene to build the sex-specific splicing element. A synthetic start codon with Kozak sequence was followed by constitutive baculovirus promoter Hr5IE1 to initialize the reading frame. One nucleotide insertion at the beginning of exon4 and the stop codon elimination on exon5b were engineered to open the frame. The endogenous intron4 and intron6 were too huge to work for plasmid construction, therefore, the truncated intron sequences were used to build the Aadsx splicing module. The overlapping open reading frame was designed to express DsRed and EGFP individually via ribosomal frameshift which was regulated by sex-specific RNA splicing. The SV40 poly(A) signal was a transcriptional termination signal. The schematic map of the vector plasmid coding the male splicing product is shown as Vector_1174D in FIG. 15A. The results showed that all the EGFP-expressed mosquitoes were male. However, no DsRed-expressed mosquitoes were observed (Table 2).

TABLE 2
Results of Male Splicing Expression System in Sex Sorting

| | |
|---|---|
| GFP+ larvae | GFP− larvae |

| | Male | Female | Male | Female |
|---|---|---|---|---|
| G0 | 55 (100%) | 0 | — | — |
| G1 | 43 (100%) | 0 | 171 | 173 |
| G2 | 142 (100%) | 0 | 18 | 146 |
| G3 | 137 (100%) | 0 | 14 | 169 |
| G4 | 378 (100%) | 0 | 9 | 311 |
| G5 | 99 (100%) | 0 | 0 | 96 |

Based on the result of the male splicing product, a two-marker expression system was further designed for sex sorting. The constitutive baculovirus promoter OpIE-2 was used to express DsRed marker in both genders. The constitutive baculovirus promoter Hr5IE1 and the sex-specific splicing element from Aadsx were used to regulate male-specific EGFP expression. The truncated introns (intron4 and intron6) and endogenous female specific exons (exon5a and exon5b) were incorporated into the splicing module to remain the sex-specific RNA splicing. The schematic map of the vector plasmid coding a two-marker expression system is shown as vector_1171I in FIG. 15B. The results showed that all the DsRed positive mosquitoes were female, on the other hand, the mosquitoes with both markers (DsRed and EGFP) were male. The results are shown in Table 3.

TABLE 3
Results of A Two-Marker Expression System in Sex Sorting

| | |
|---|---|
| Both GFP and DsRed | DsRed only |

| | Male | Female | Male | Female |
|---|---|---|---|---|
| G0 | 68 | 0 | 0 | 77 |
| G1 | 96 | 0 | 0 | 92 |
| G2 | 291 | 0 | 0 | 296 |
| G3 | 287 | 0 | 0 | 291 |
| G5 | 582 | 0 | 0 | 585 |
| Sex % | 100% | 0% | 0% | 100% |

### Example 2—Engineering SEPARATOR

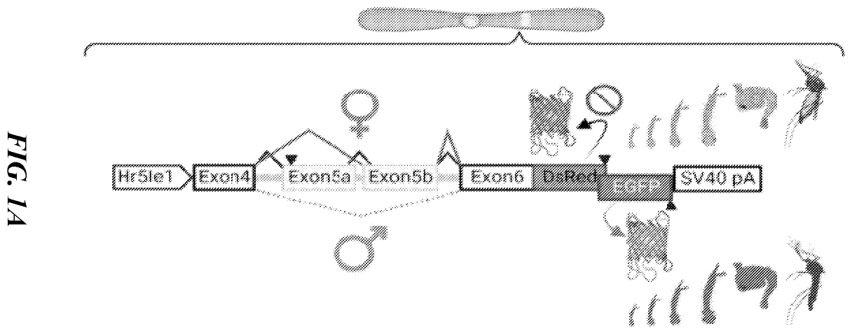

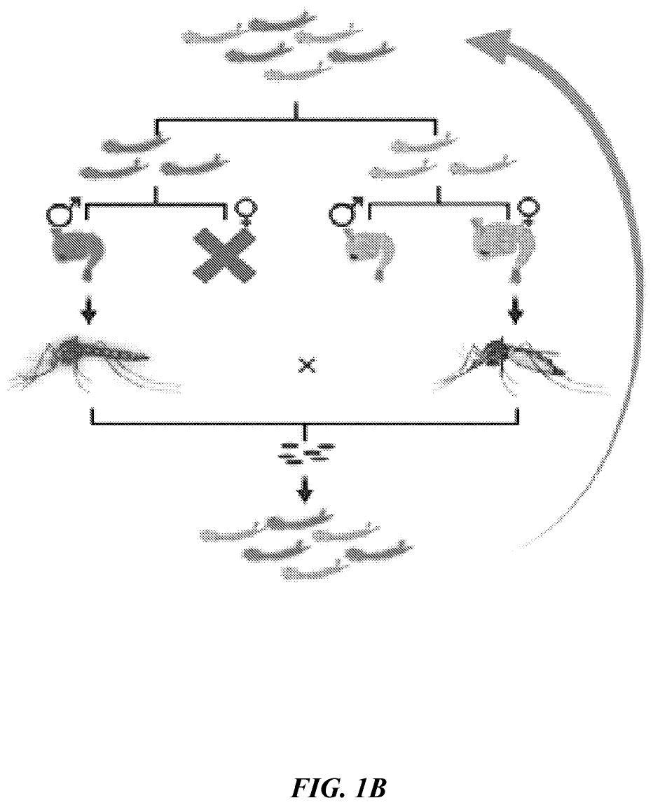

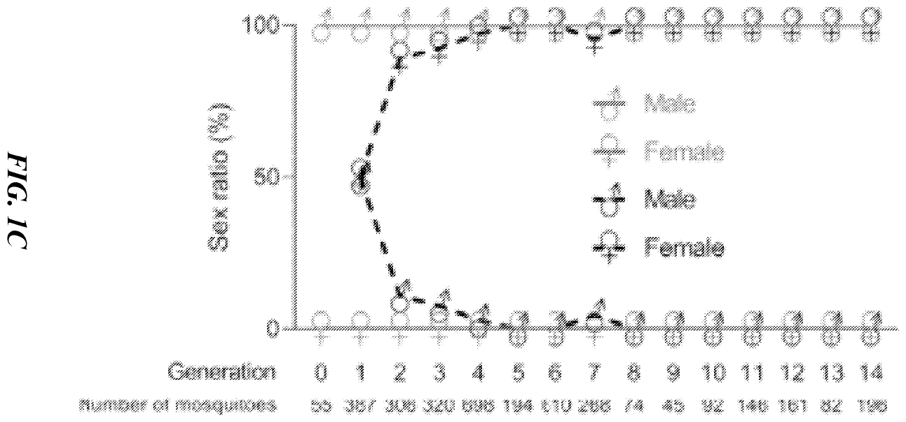

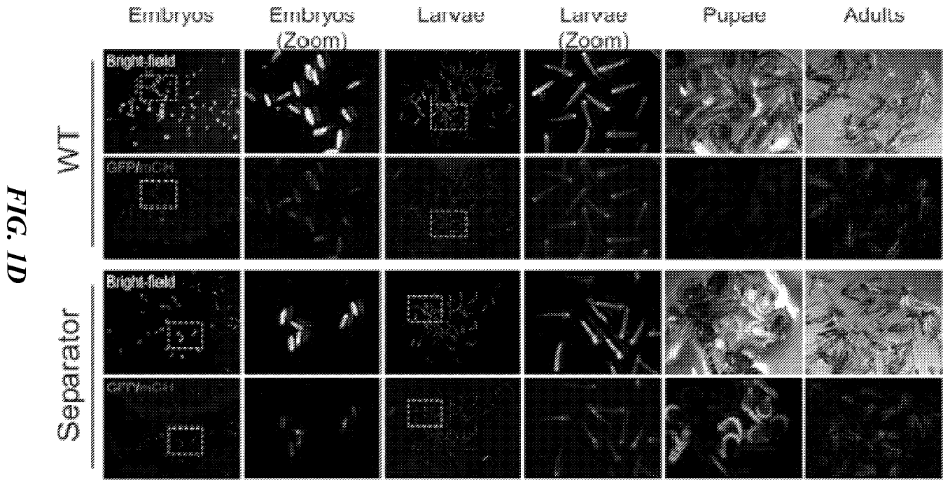

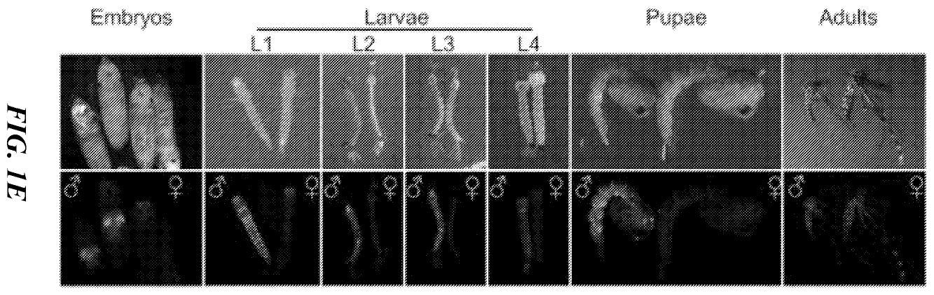

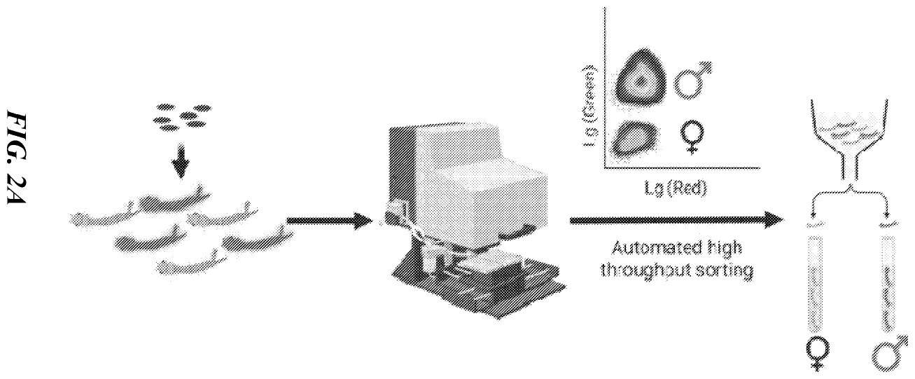

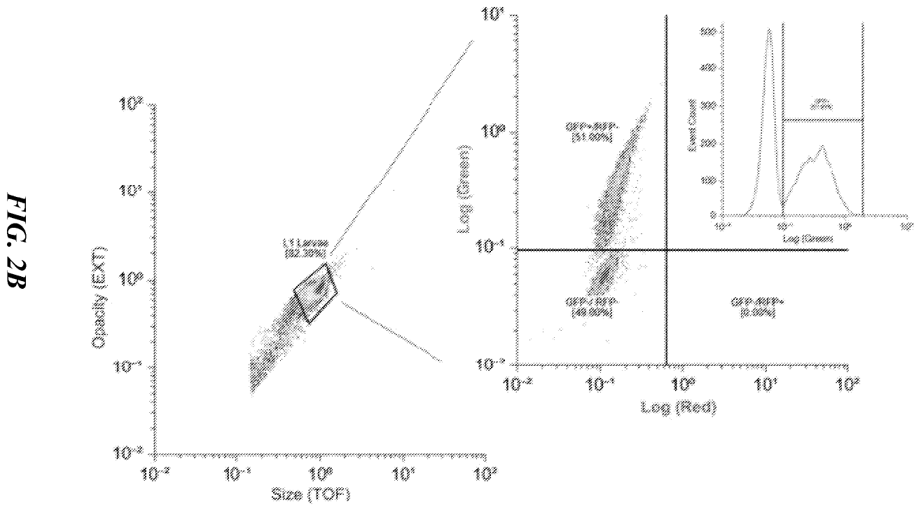

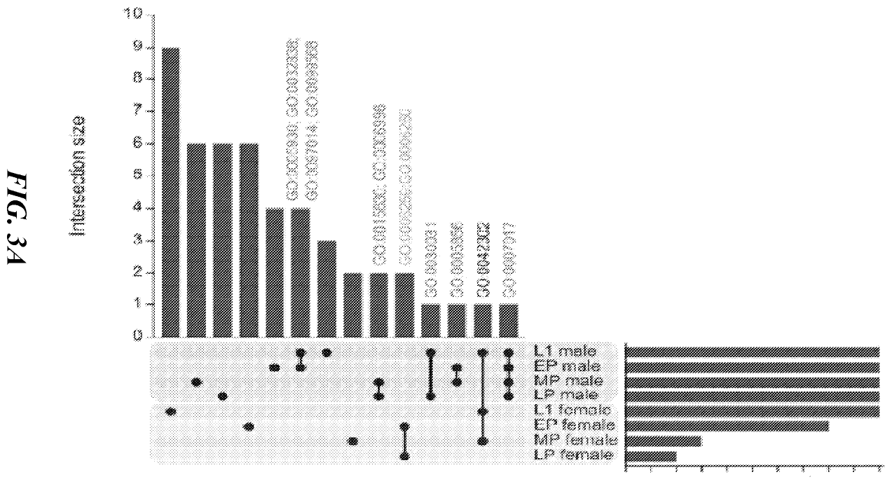

To generate SEPARATOR a sex-specific alternatively spliced intron derived from the Ae. aegypti doublesex (AaeDsx) gene was utilized (FIG. 1A and FIG. 5A). Dsx is a highly conserved transcription factor involved in sex determination of insects. In Ae. aegypti the male specific AaeDsx intron is ˜26.5 kb which is a bit large to work with. Therefore, this intron was truncated by preserving splicing factor binding sites including Tra/Tra-2 and RNA binding protein 1 (RBP1) binding sites to retain the sex-specificity of this intron. This resulted in a smaller AaeDsx intron of 4.5 kb in size (FIG. 1A and FIG. 5A). The reading frame was initiated by adding a start codon with a Kozak sequence, expressed using a constitutive Hr5IE1 AcMNPV baculovirus promoter previously shown to work in many species. To open the reading frame, nine stop codons located in endogenous exon 5b were excluded. The coding sequences for EGFP and DsRed were strategically designed to overlap, allowing for their expression in a sex-specific manner. The DsRed coding sequence was designed to be in-frame with a female-specific product (exon4, engineered exon5b, and exon6) to control female-specific DsRed expression. In addition, the male-specific splicing product, involving exon 4 and exon 6, was designed to be in-frame with the EGFP coding sequence (FIGS. 5A-5C).

The SEPARATOR construct was then introduced into the mosquito genome to generate a genetic sex-sorting strain via the piggyBac transposon. The intended plan was to ensure that all mosquitoes expressing GFP would be male, while those expressing DsRed would be female. Interestingly, the results following microinjection revealed that all 55 EGFP-expressed larvae were male at the pupal stage in G0 (Table 4). However, no DsRed-expressed larvae were observed in G0. All the pupae from G0 were sexed and resulting adults were crossed with wild-type mosquitoes, and stable transgenic lines were selected by the fluorescence marker (G1). In G1-onward, similar results were observed, where 100% of the EGFP-positive larvae were male.

TABLE 4
The sex sorting of SEPARATOR mosquitoes during the initial 15 generations

| | | GFP+ | | | GFP− | | |
|---|---|---|---|---|---|---|---|
| | Number of | Number | Number | | Number | Number | |
| Number of | counted | of | of | | of | of | |
| generation | mosquitoes | male | female | ♂(%) | male | female | ♀(%) |
| G0 | 55 | 55 | 0 | 100 | NA | NA | NA |
| G1 | 387 | 43 | 0 | 100 | 171 | 173 | 50.29 |
| G2 | 306 | 142 | 0 | 100 | 18 | 146 | 89.02 |
| G3 | 320 | 137 | 0 | 100 | 14 | 169 | 92.35 |
| G4 | 698 | 378 | 0 | 100 | 9 | 311 | 97.19 |
| G5 | 195 | 99 | 0 | 100 | 0 | 96 | 100 |
| G6 | 610 | 320 | 0 | 100 | 0 | 290 | 100 |
| G7 | 268 | 158 | 0 | 100 | 5 | 105 | 95.45 |
| G8 | 74 | 42 | 0 | 100 | 0 | 32 | 100 |
| G9 | 45 | 24 | 0 | 100 | 0 | 21 | 100 |
| G10 | 92 | 46 | 0 | 100 | 0 | 46 | 100 |
| G11 | 146 | 79 | 0 | 100 | 0 | 67 | 100 |
| G12 | 161 | 83 | 0 | 100 | 0 | 78 | 100 |
| G13 | 82 | 45 | 0 | 100 | 0 | 37 | 100 |
| G14 | 196 | 101 | 0 | 100 | 0 | 95 | 100 |

*NA means not analyzed

To validate the sex-specific splicing pattern of the AaeDsx splicing module, a comprehensive analysis was carried out utilizing reverse transcription polymerase chain reaction (RT-PCR) and RNA sequencing. A total of fifty EGFP-positive L1 larvae and fifty EGFP-negative L1 larvae were collected for further analysis. To perform RT-PCR, primers designed to target the 3′ end of the Hr5IE1 promoter and the 5′ end of the EGFP sequence were utilized (FIG. 5A). Subsequently, Sanger sequencing was carried out to analyze the obtained PCR products. The results showed that the utilization of truncated intron 4, engineered exon 5b, intron 6, and exon 6 sequences in RNA splicing exhibited sex-specificity (FIG. 5B). Both Sanger sequencing and RNA sequencing (RNA-seq) analysis yielded comparable results, aligning with the anticipated splicing patterns (FIG. 5B and FIG. 6). The sex-specific RNA splicing pattern indicated the female splicing product being in-frame with the DsRed coding sequence, while the male splicing product resulted in (−1) frameshift, leading to the DsRed coding sequence being out of the frame and in-frame with the EGFP coding sequence. Interestingly, both the RT-PCR and RNA-seq analyses revealed that the predominant products observed in females comprised exon4, exon5b, and exon6 (FIGS. 5B-5C and Table 5). Notably, these exons were found to be in-frame with the DsRed coding sequence (FIG. 5B and FIG. 6). Therefore, it can be inferred that the expression level of transcripts specific to females and the splicing pattern of female-specific products do not present any obstacles to DsRed expression. However, DsRed signals were not observed in the transgenic larvae.

Transgene integration sites were determined (FIG. 7) and a homozygous line was generated. To do this, larvae were sorted by fluorescence and separated into two groups, EGFP-positive and EGFP-negative, then the sex ratio of these two groups was examined at the pupal/adult stage. The EGFP-positive males were crossed to EGFP-negative females to enrich for homozygotes (FIG. 1B). In 15 generations, a total of 3635 EGFP+ larvae were manually screened, and the resulting sex was confirmed at the pupa and adult stage. Remarkably, 100% of the GFP+ larvae were male (FIG. 1C and Table 4). In summary, the results demonstrate that the SEPARATOR technology is an efficient and effective way of separating male (EGFP+) and female mosquitoes (EGFP−), which could have important implications for broad adaptation of SIT for mosquito control.

TABLE 5
FPKM of each exon in SEPARATOR mosquitoes

| | GFP | GFP | GFP | GFP | GFP | GFP |
|---|---|---|---|---|---|---|
| ID | negative-1 | positive-1 | negative-2 | positive-2 | negative-3 | positive-3 |
| DsRed1 | 240.51 | 145.18 | 397.77 | 116.82 | 311.37 | 125.48 |
| EGFP | 361.7 | 223.68 | 617.99 | 190.38 | 488.88 | 199.2 |
| Exon4 | 214.34 | 144.89 | 306.32 | 111.19 | 237.62 | 121.81 |
| Exon5a | 69.8 | 17.67 | 112.89 | 11.51 | 84.42 | 15.45 |
| Exon5b | 220.52 | 18.91 | 347.44 | 16.79 | 259.89 | 17.41 |
| Exon6 | 200.08 | 131.48 | 335.1 | 101.58 | 237.35 | 108.47 |
| 4_6_average | 207.21 | 138.18 | 320.71 | 106.39 | 237.49 | 115.14 |
| 4_fraction | 1.03 | 1.05 | 0.96 | 1.05 | 1 | 1.06 |
| 5a_fraction | 0.34 | 0.13 | 0.35 | 0.11 | 0.36 | 0.13 |
| 5b_fraction | 1.06 | 0.14 | 1.08 | 0.16 | 1.09 | 0.15 |
| 6_fraction | 0.97 | 0.95 | 1.04 | 0.95 | 1 | 0.94 |

### Example 3—Measuring Efficacy of Sex Sorting at Scale

To evaluate the applicability of SEPARATOR during the entire life cycle of mosquitoes, the timing of EGFP expression was further explored. Strong EGFP signals were found to be expressed from late embryo all the way through to the adult stage of the mosquitoes (FIG. 1D-1E). In addition, when compared to the wild-type control (Liverpool), the EGFP intensity of SEPARATOR mosquitoes is strong enough to differentiate between EGFP-positive (male) and EGFP-negative (female) in a pooled condition from the early stage of mosquito's life cycle (FIG. 1D). Based on these results, it can be concluded that SEPARATOR is a powerful and robust system for mosquito sex sorting.

To evaluate the suitability of SEPARATOR for automated sex sorting, fluorescence-based flow cytometry sorting was conducted on batches of several thousand first instar larvae using COPAS®. This generated a fluorescence diagram where male larvae expressing EGFP formed a distinct cluster, clearly separate from the GFP negative female larvae (FIGS. 2A-2B and FIG. 8). The EGFP-positive larvae were selected and sorted in pure mode at flow rates ranging from 20 to 70 objects per second. Even at this high flow rate. 70-80% of the EGFP-positive larvae were recovered, resulting in sorting speeds of 740 larvae/minute (Table 6). In total, 108,570 larvae were sorted using COPAS® from generations G7 to G9. After a single sorting, a single instance of 0.1 to 0.45% contamination of EGFP-negative (female) larvae was observed within the male population (Table 6). While slower flow rates could potentially yield a more complete recovery of males and minimize or eliminate contamination with females, the high sorting speed represents a compromise between recovery rates and the production speed required for mass production. To address the issue of female contamination, additional measures were implemented. Through quality control sorts, the contamination rate of EGFP-negative larvae (females) was successfully reduced to 0.01-0.03% (Table 6). This was accomplished by subjecting the sorted larvae, a total of 78,861 individuals at G9, to a second round of sorting using the COPAS®. This second sorting phase specifically targeted objects that deviated from the EGFP-positive gate, employing the “enrich mode” for enhanced precision. These experiments were conducted using an older SELECT COPAS® instrument model from 2005. Utilizing a more modern COPAS® instrument with an upgraded laminar flow and electronic controls, is expected to further reduce these reported female contamination rates. Taken together, this method efficiently and effectively sorted a large number of larvae, providing valuable insights into the performance of the sex sorting system.

TABLE 6
Summary of results from COPAS ® sorting experiments

| | | | | | | sorted GFP+ | Female | |
|---|---|---|---|---|---|---|---|---|
| | Sorting | | | | | larvae | contamination | |
| | iterations | Total | GFP+ | GFP− | % gated | (% of total | (% of sorted GFP+ | Sorting |
| Generation | count | larvae | larvae | larvae | objects | GFP+) | larvae) | time |
| G7 | Single | 11348 | 5797 | 5551 | 45.70% | 4720 (81.4%) | 6 (0.16%)a | 10 min |
| G8 | Single | 9094 | 4582 | 4512 | 41.20% | 3299 (72%) | 9 (0.23%)b | 8 min |
| | Single | 8267 | 3835 | 4432 | 40.40% | 2848 (74%) | 3 (0.1%)c | |
| G9 | Double | 27769 | 14121 | 13648 | 41.30% | 9304 (65.8%) | 42 (0.45%)d | 14 min |
| | (1st) | | | | | | | |
| | Double | | | | | | 3 (0.03%)e | |
| | (2nd) | | | | | | | |
| | Double | 26456 | 13301 | 13155 | 41.70% | 9301 (70%) | 13 (0.13%)d | 15 min |
| | (1st) | | | | | | | |
| | Double | | | | | | 1 (0.01%)e | |
| | (2nd) | | | | | | | |
| | Double | 25636 | 12541 | 13095 | 41.70% | 8879 (70.8%) | 21 (0.23%)d | 13 min |
| | (1st) | | | | | | | |
| | Double | | | | | | 1 (0.01%)e | |
| | (2nd) | | | | | | | |

a: In the course of this experiment, female contaminants were observed during the pupal stage. The evaluation of the survival rate during larval culture, based on three test cultures consisting of 200 sorted larvae each, revealed a survival rate of 72%. Consequently, the contamination was estimated to be six individuals out of a total of 3831 larvae. This results in a female contamination rate of 0.16% within a male population.
In subsequent experiments, contamination was directly measured using quality control COPAS ® sorts, ensuring accurate assessment of contamination levels.
b: 9 contaminants out of 3299 + 607 additionally collected larvae. This results in a female contamination rate of 0.23% within a male population.
c: Perform another round of sorting to validate the error rate.
d: First sorting. GFP-negative larvae are separated using Enrich mode and subsequently verified through microscopy
e: Second sorting involves selecting from the GFP-positive population obtained in the first sorting procedure.

### Example 4—Sex-Enriched Genes Identification

Previously, sex determination in mosquitoes during the larvae stage has presented challenges, necessitating the reliance on sex-specific morphological characteristics in pupae and adults for precise identification. Consequently, earlier transcriptome analyses primarily concentrated on investigating the sex-related aspects of pupae and adult stages. While there have been some limited studies successfully distinguishing sexes at the L3 and L4 larval stages by assessing the expression of the male determinant factor, Nix, this method requires individual PCR testing of larvae to detect Nix and determine the sex of individual mosquitoes before proceeding with RNA-seq analysis.

In order to gain valuable insights into the molecular mechanisms controlling sex determination and differentiation during the early developmental stages of Ae. aegypti mosquitoes, SEPARATOR was employed to separate male and female L1 larvae. Subsequently, RNA sequencing was conducted to identify genes exhibiting sex-specific expression patterns. Using the collected data, a comprehensive analysis of differential gene expression (DGE) was conducted. The findings revealed that, at the early L1 larvae stage, 1082 genes exhibited male-enriched expression patterns, while 634 genes exhibited female-enriched expression patterns (FIG. 9C). Subsequently, the sex-enriched genes were subjected to enrichment analysis. Among the male-enriched genes, two distinct clusters emerged based on Gene Ontology (GO) terms. The first cluster was related to cilium and microtubule formation (including terms such as cilium, axoneme, microtubule-based process, and cell projection), while the second cluster was associated with cuticle formation (FIG. 9D). Moreover, among the female-enriched genes, several distinct clusters emerged based on GO terms, including immune response, metabolic processing, and cuticle formation (FIG. 9E).

Following RNA-seq analysis of L1 larvae, the disclosed transcriptomic data at the L1 larvae stage were compared with data from the L3, L4, pupae, and adult stages of mosquitoes. To determine the overlap between the SEPARATOR set and the other six comparisons, an UpSet plot was generated to represent shared sex-enriched genes across all developmental stages (FIG. 11 and FIG. 12). In the present analysis, a significant number of genes were identified that were not detected in any of the comparisons performed by previous studies. This finding suggests that some of these genes may represent early expressed genes that are later turned off in subsequent stages, making them difficult to identify in previous studies.

In order to further explore the investigation of sex-enriched genes during various developmental stages of mosquitoes, GO enriched analysis on the list of sex-enriched genes was performed at different developmental stages. However, a limited number of genes were observed displaying sex-enriched patterns specifically in the L3 and L4 larval stages. Furthermore, when analyzing Matthews's RNA-seq dataset, no significant difference was found in the expression level of a well-known male determinant factor, Nix, between L3 males and L3 females. This discrepancy could potentially be attributed to the lower sequencing depth in Matthews's RNA-seq data (approximately 7 million reads per sample) compared to the present L1 stage dataset (approximately 25 million reads per sample) (FIG. 10). Additionally, to ensure consistency with samples from other stages, the dataset derived from adult mosquitoes that had been dissected and isolated to obtain independent samples was excluded. As a result, GO enriched analysis on the sex-enriched gene list was conducted for the L1 larvae and pupae stages of mosquitoes. Disclosed results indicate a consistent enrichment of genes involved in cilium and microtubule formation in males throughout the developmental stages of mosquitoes, ranging from L1 larvae to late pupae. Notably, cytoskeleton organization-related GO terms were identified during the early to mid pupae stages. Moreover, GO terms associated with spermatid development and sperm DNA condensation were identified during the late pupae stage of mosquito development (FIG. 3 and FIG. 13). These results suggest that sperm development is a continuous process from the early developmental stage (L1 larvae) to the late developmental stage (late pupae) in mosquitoes. For female-enriched genes, a notable emphasis on GO terms was observed to be associated with DNA replication and DNA repair specifically in female pupae (FIG. 3 and FIG. 13).

Previously, it was challenging to determine the sex of larvae. As a result, previous transcriptome analyses relied on separating sexes after the pupae stage, which caused the loss of sex-specific samples during early developmental stages. Herein, the disclosed sex-specific RNA-seq results were utilized to be compared with a previously collected dataset of variable developmental stage RNA-seq data. To assess the expression of sex-specific genes during early development (prior to the pupae stage), the genes identified through mfuzz clustering analysis were initially analyzed, specifically focusing on those designated as L1 or L2-L4 specific. Cluster 17 consisted predominantly of genes expressed in L1, while cluster 1 encompassed genes expressed in L2-L4 (FIGS. 14A-14B). Using a membership cutoff of 0.75, cluster 17 was found to contain 268 genes and cluster 1 was found to contain 383 genes. Among these, 73 (27%) and 134 (35%) were determined to be sex-specifically expressed. To expand the present investigation beyond the mfuzz clusters, genes that exhibited no expression (TPM values below 1) in carcass, testes, ovary, or pupae, but displayed TPM values above 1 or 10 in first instar larvae were examined. These were considered early-expressed genes that were not detected at later stages. 210 and 93 such genes were identified in the respective datasets. Among these, 76 (36%) and 46 (49%) were identified as sex-specifically expressed.

Drosophila melanogaster

### Molecular Cloning

All genetic constructs were produced utilizing the Gibson enzymatic assembly. The construct 795G was created using a pre-existing plasmid containing piggyBac, attB-docking sites, and an Opie2 promoter regulating dsRed. This plasmid was subsequently linearized with XhoI and NotI enzymes. The Hr5Ie1 promoter, along with eGFP were cloned into the linearized plasmid to make 795G, which serves as the control plasmid. To generate female-specific dsRed (795H-K), the plasmid 795G was linearized with AvrII and BamHI to allow insertion of introns into dsRed. Alternatively, to generate female-specific eGFP (795L-O), 795G was linearized using MluI and BsrGI to insert introns into eGFP. The traF introns from D. melanogaster, D. suzukii, C. capitata, or A. ludens were amplified from their respective genomic DNA using the primers listed in Table 7.

### Reverse Transcription PCR (RT-PCR) of the Female-Specific Splicing Transcripts

To access the splicing transcripts of four traF introns, female- and male-specific dsRed mRNA were screened. Total RNA often virgin females or males from w-, 795G, H, I, J, and K were extracted using the miRNeasy Tissue/Cells Advanced Kits (Qiagen). DNase treatment is done using the TURBO™ DNA-free (Invitrogen), and followed by the cDNA synthesis using the RevertAid First Strand cDNA Synthesis Kit (Thermo Scientific™). The genomic DNA (gDNA) was amplified using primers 795.s2F and 795.s2R (Table 7), and the cDNA was amplified using primers 795.s3F and 795.s1R. The gDNA samples were run on 1% TAE agarose gel, and the cDNA samples were run on a 2% TAE agarose gel.

TABLE 7
Primer sequences

| | | Primer | | |
|---|---|---|---|---|
| | | name | Primer sequence (5′-3′) | SEQ ID NO |
| geno- | DstraF | 1122.S1F | cggctaggttaaacgagtagtgtccacctc | SEQ ID NO: |
| typeing | | | | 14 |
| introns | | 1122.S2R | GTAATTTACTAACGTAGAAATCCTGTGCTGGCAC | SEQ ID NO: |
| | | | | 15 |
| | CctraF | 825A | AAAAATAAGGTCAGCAGCCATGAC | SEQ ID NO: |
| | | | | 16 |
| | | 825B | GTCATGGCTGCTGACCTTATTTTT | SEQ ID NO: |
| | | | | 17 |
| | | 825C | AGACATTCCAAATTCAAGTTAACAAATTAAT | SEQ ID NO: |
| | | | | 18 |
| | | 825D | ATTAATTTGTTAACTTGAATTTGGAATGTCT | SEQ ID NO: |
| | | | | 19 |
| | AltraF | 1168-Tra- | CTTGCTCTGGAGTCCGTTAAA | SEQ ID NO: |
| | | gDNA-R1 | | 20 |
| | | 1168-Tra- | CCTGAGCAGATAACCCTCTTTC | SEQ ID NO: |
| | | gDNA-F2 | | 21 |
| | | 1168-Tra- | GGTCGTTTCGGTAACGTAGAA | SEQ ID NO: |
| | | gDNA-F3 | | 22 |
| | | 1168-Tra- | GTCATCAGTTGTCGGCATTTAC | SEQ ID NO: |
| | | gDNA-R2 | | 23 |

| | | | |
|---|---|---|---|
| Cloning primers | 795G2.c1R | ccgcaacctgtctctggtgGGATCCatggtgcgctcctccaagaacgtcatcaag | SEQ ID NO: |
| | | | 24 |
| | 795K | acgccatccaaccgccgccgcaacctgtctctggtgATGgtaattttaaaagcat | SEQ ID NO: |
| | | atttttttc | 25 |
| | 795I | acgccatccaaccgccgccgcaacctgtctctggtgATGgtaattttaaaagcat | SEQ ID NO: |
| | | atttttttc | 26 |
| | 1122J.c1F | aattcaacgcacacttattacgtgaggtaccgcgcccatactcggtggcctcccc | SEQ ID NO: |
| | | ac | 27 |
| | 795L.c1F | ggccgactgttttcgtatccgctcaccaaacgcgtttttgcattaacattgtatg | SEQ ID NO: |
| | | tcggc | 28 |
| | 795L.c2R | tcaaagaaaaaaatatgcttttaaaattacCATtttgTATTgtcacttggttgtt | SEQ ID NO: |
| | | cacga | 29 |
| | 795L.c3F | tcgtgaacaaccaagtgacAATAcaaaATGgtaattttaaaagcatatttttttt | SEQ ID NO: |
| | | tt | 30 |
| | 795L.c4R | ACCCCGGTGAACAGCTCCTCGCCCTTGCTctatagataccatagatgtat | SEQ ID NO: |
| | | ggattagtat | 31 |
| | 795L.c5F | atactaatccatacatctatggtatctatagAGCAAGGGCGAGGAGCTGTTCA | SEQ ID NO: |
| | | CCGGGGT | 32 |
| | 795M.c2R | ATCAGATCGGTTATACTATATAGTGGGTACCATtttgTATTgtca | SEQ ID NO: |
| | | cttggttgttcacga | 33 |
| | 795M.c3F | tcgtgaacaaccaagtgacAATAcaaaATGGTACCCACTATATAGTATA | SEQ ID NO: |
| | | ACCGATCTGAT | 34 |
| | 795M.c4R | CACCCCGGTGAACAGCTCCTCGCCCTTGCTCTATGTGAAAA | SEQ ID NO: |
| | | GAGTGTGCGGTTAGTCAAT | 35 |
| | 795M.c5F | ATTGACTAACCGCACACTCTTTTCACATAGAGCAAGGGCGA | SEQ ID NO: |
| | | GGAGCTGTTCACCGGGGTG | 36 |
| | 795N.c2R | tctgatccgatcgaatatgtgtatatatacCATtttgTATTgtcacttggttgtt | SEQ ID NO: |
| | | cacga | 37 |
| | 795N.c3F | tcgtgaacaaccaagtgacAATAcaaaATGgtatatatacacatattcgatcgga | SEQ ID NO: |
| | | tcaga | 38 |
| | 795N.c4R | CCCCGGTGAACAGCTCCTCGCCCTTGCTctacgtggaagtggaagaag | SEQ ID NO: |
| | | aggtgttaatcac | 39 |
| | 795N.c5F | gtgattaacacctcttcttccacttccacgtagAGCAAGGGCGAGGAGCTGTTC | SEQ ID NO: |
| | | ACCGGGG | 40 |
| | 7950.c2R | ttcataaaataaaatgtaggttacaattacCATtttgTATTgtcacttggttgtt | SEQ ID NO: |
| | | cacga | 41 |
| | 7950.c3F | tcgtgaacaaccaagtgacAATAcaaaATGgtaattgtaacctacattttatttt | SEQ ID NO: |
| | | atgaa | 42 |
| | 7950.c4R | CACCCCGGTGAACAGCTCCTCGCCCTTGCTctgtgggcacgatgatttt | SEQ ID NO: |
| | | ttatattagta | 43 |
| | 7950.c5F | tactaatataaaaaatcatcgtgcccacagAGCAAGGGCGAGGAGCTGTTCA | SEQ ID NO: |
| | | CCGGGGTG | 44 |
| DsRed splicing | 795.s2F | CAATTGTGGCGTTTACAGCATTTGTTATACACACAGAACTC | SEQ ID NO: |
| PCR | | | 45 |
| | 795.s2R | gaacagcatctgttacagcgacacaacatg | SEQ ID NO: |
| | | | 46 |
| | 795.s3F | CGATTCATCCTAGGctacaggaacaggtg | SEQ ID NO: |
| | | | 47 |
| | 795.s1R | catccaaccgccgccgcaacctgtc | SEQ ID NO: |
| | | | 48 |

### Rearing and Fly Transgenesis

Transgenic flies were maintained under standard conditions at 25° C. with a 12H/12H light/dark cycle and fed on the Old Bloomington Molasses Recipe. Embryonic injections were performed in the lab following the standard injection protocol. Plasmids diluted to 300-350 ng/μL in water were inserted at P{CaryP}attP40 on the 2nd chromosome (Bloomington #25709). Recovered transgenic lines were balanced on the 2nd chromosome using a single chromosome balancer line w1118: CyO/sna[Sco]. Multiple independent lines were obtained for each plasmid and tested for sex-specific fluorescence. Homozygous transgenic lines containing two copies of the transgene were used to assess the sex-sorting efficiency. Sex-sorting lines with CctraF introns 795H and 795L are deposited at the Bloomington Drosophila Stock Center (BDSC #pending).

### Genetics and Sex Selection

To assess the fluorescent sex selection efficiency, ten virgin females were crossed to ten males in a fly vial. The parental flies were flipped into a fresh vial every 12 hrs, and the numbers of the laid embryos were scored. After hatching, the larvae or pupa were scored and transferred to different vials based on their fluorescent markers. The sex and the fluorescent markers of the adult offsprings were recorded after eclosion. Flies were scored using a Leica M165FC fluorescent stereomicroscope. Images were taken using a View4K camera. Each genetic cross was set up five times using different parental flies.

### Fitness Estimation

The fitness of the sex-sorting strains is assessed based on two parameters: the rate of egg-hatching (from embryos to larvae) and the rate of adult survival (from larvae to adult). To evaluate the egg-hatching rate, flies are allowed to lay embryos in fly vials for a duration of 24 hrs, and the number of eggs laid in each vial is recorded. After 24 hrs of egg laying, the number of larvae is recorded. To assess the adult survival rate, the number of both female and male adult flies that successfully eclosed is recorded.

### Statistical Analysis

Statistical analysis was performed in Prism9 by GraphPad Software, LLC. Three to five biological replicates were used to generate statistical means for comparisons.

### Example 5—Development of Female-Specific Expression of the Fluorescent Protein

To engineer female-specific expression of a reporter for positive selection of females, the sex-specific alternative splicing of a conserved sex-determination gene was exploited. In D. melanogaster, the transformer (tra) intron between exons 1 and 2 is spliced out in females, resulting in a functional tra protein. In males, alternative tra splicing results in a premature stop codon that terminates the tra protein. This female-specific alternative splicing mechanism occurs not only in Drosophila but also in Ceratitis and Anastrepha, suggesting that it is highly conserved in Dipterans (FIG. 16A, FIGS. 20A-20B, FIG. 21). Consequently, the traF from D. melanogaster, D. suzukii, C. capitata, or A. ludens were inserted into the fluorescent protein coding sequences to test for female-specific fluorescent protein expression (FIG. 16B).

Two sets of dual fluorescent marker constructs were generated encoding a fluorescent marker for both sexes and a female-specific fluorescent marker (FIG. 16C). The constructs were cloned into a plasmid containing an attP recombination site and a piggyBac (PB) transposable element. Set 1 constructs have an eGFP fluorescence expressed under a ubiquitous promoter Hr5Ie1 (Hr5Ie1-eGFP) as the selectable marker for the transgene. To promote constitutive expression of female-specific fluorescent proteins, another ubiquitous promoter Opie2 was used to express dsRed and traF was inserted immediately downstream of the ATG translational start codon of dsRed (Opie2-ATG-traF-dsRed). Constructs in set 2 have the opposite marker configuration, with traF inserted downstream of the ATG translational start codon of eGFP under promoter Hr5Ie1 (Hr5Ie1-ATG-traF-eGFP) for the female-specific fluorescent expression and Opie2-dsRed as the selectable marker for the transgene. In total, nine constructs were created: a control construct (795G) and eight experimental constructs, with four constructs in each set (FIG. 16D).

### Example 6—DntraF, DstraF, and CctraF Resulted in Female-Specific Fluorescence

The transgene integration site can impact gene expression, so it was opted to integrate all nine constructs into the same site through phiC31 attP integration on the second chromosome (BDSC #25709). Nine homozygous transgenic strains were established. Six constructs that harbor DmtraF, DstraF, and CctraF resulted in female-specific fluorescence (795H, I, J, L, M, and N, FIGS. 17A-17C). These results indicate that inserting the traF into the coding sequence of the fluorescence proteins can result in female-specific fluorescence. However, for constructs harboring AltraF (795K and O), both females and males exhibited the intended female-specific fluorescence (FIGS. 17A-17C). This result suggests that AltraF is spliced out in both females and males rather than in a female-specific manner.

To validate the alternative splicing variants, adult flies were collected for RT-PCR analysis to obtain the fluorescent protein transcripts. Primers were designed to anneal to the 5′ UTR region at the 3′ end of the Opie2 promoter, and the 3′ end of the dsRed sequence (Table 7). Multiple bands were obtained from the RT-PCR samples for CctraF males, and both sexes of DmtraF and DstraF (FIGS. 22A-22B). Sequencing of these bands indicates that CctraF, DmtraF, and DstraF resulted in functional dsRed expression explicitly in females, while AltraF had dsRed expression in both males and females (FIG. 22C). The molecular results obtained from RT-PCR analysis were consistent with the observations in the flies, confirming that CctraF, DmtraF, and DstraF exhibited female-specific splicing in D. melanogaster. This outcome demonstrates the feasibility of this fluorescent sex-sorting approach, as these female-specific splicing events allow for the positive selection of either sex.

### Example 7—Confirmation of Sex-Specific Fluorescence at Multiple Developmental Stages

Next, the intensity and sex specificity of the fluorescence was evaluated over multiple life stages. The six constructs (795H, I, J, L, M, and N) that exhibited female-specific fluorescent expression were evaluated. Female-specific fluorescence was observed as early as in the first instar larvae (L1) life stage in both CctraF transgenic lines: 795H and 795L (FIG. 17A, FIG. 18). Female-specific fluorescence was also observed in the third instar larvae (L3) of DmtraF 7951 and CctraF 795L, and the pupal stage for DmtraF 795M and DstraF 795N. Despite the identical introns in the DmtraF 7951 and DmtraF 795M and the CctraF 795L and DstraF 795N strains, female-specific fluorescence was detected earlier in strains with the female-specific dsRed marker. This result is presumably due to the deeper tissue penetrance and lower auto-fluorescence of red fluorescent protein (RFP). Notably, the intensity of the female-specific fluorescence varies among introns. The CctraF exhibits the highest brightness, followed in order of brightness by DmtraF and DstraF (FIG. 18). This is unexpected as CctraF is an exogenous/non-native intron for D. melanogaster, potentially hindering successful intron recognition and splicing efficiency.

### Example 8—Assessing the Fitness of the Sex-Sorting Strains

Strain fitness is essential for scalability. Fluorescent proteins have documented fitness costs to genetically engineered organisms, but it was expected that including traF in their coding sequences would minimally affect the fitness of the sex-sorting strain. Therefore, the egg hatching and larval to adult survival rate of all eight homozygous sex-sorting strains were compared to a control strain (795G) containing fluorescent reporters lacking traF introns. The finding indicates that there is a significantly higher hatching rate in flies harboring Opie2-ATG-traF-dsRed constructs when compared to 795G control (FIGS. 19A-19B). This effect could possibly be attributed to the fitness cost associated with dsRed functioning as a tetramer. With the inclusion of traF introns, the expression of dsRed occurs at a reduced level. As a consequence, the fitness cost is diminished, which, in turn, leads to a higher hatching rate. Lower larvae to adult survival was observed only in the CctraF-dsRed 795H strain (p<0.05, Student's t-test with equal variance, FIGS. 19A-19B). These results suggest that the traF intron does not impose substantial fitness costs on the strain, making them suitable for potential large-scale insect population control projects.

Ceratitis capitata

### Plasmid Design and Construction

Both 795H1 and 795K1 were constructed via Gibson assembly into an existing piggyBac plasmid containing Opie2 expressing DsRed and Hr5Ie1 expressing eGFP. This plasmid was linearized using AvrII and BamHI restriction endonucleases. The tra intron of either C. capitata or A. ludens was PCR amplified from the corresponding genomic DNA preps and then inserted into the DsRed coding sequence immediately downstream of an ATG start codon (Table 8). Complete sequence maps and plasmids are deposited at Addgene.org (#205482 for 795H1 and #205485 for 795K1).

TABLE 8
Primer Summary

| Purpose | Name | Sequence (5′-3′) | Note/SEQ ID NO |
|---|---|---|---|
| C. capitata | 825A | AAAAATAAGGTCAGCAGCCATGAC | SEQ ID NO: 49 |
| intron | 825B | GTCATGGCTGCTGACCTTATTTTT | SEQ ID NO: 50 |
| | 825C | AGACATTCCAAATTCAAGTTAACAAATTAAT | SEQ ID NO: 51 |
| | 825D | ATTAATTTGTTAACTTGAATTTGGAATGTCT | SEQ ID NO: 52 |
| A. ludens | 1168-Tra-gDNA-R1 | CTTGCTCTGGAGTCCGTTAAA | SEQ ID NO: 53 |
| intron | 1168-Tra-gDNA-F2 | CCTGAGCAGATAACCCTCTTTC | SEQ ID NO: 54 |
| | 1168-Tra-gDNA-F3 | GGTCGTTTCGGTAACGTAGAA | SEQ ID NO: 55 |
| | 1168-Tra-gDNA-R2 | GTCATCAGTTGTCGGCATTTAC | SEQ ID NO: 56 |
| DsRed splicing | Opie2_V2 | CCGCAACCTGTCTCTGGTG | SEQ ID NO: 57 |
| PCR | DsRed_B | CGATGGTGTAGTCCTCGTTGTG | SEQ ID NO: 58 |

C. capitata Rearing

The wild-type Benakeion strain of C. capitata used herein was obtained from the Saccone Lab (University of Naples “Federico II”). Adult food consisted of a mixture of yeast and glucose in equal proportions and larvae were maintained using a carrot-based diet. Flies were consistently kept in 12:12 hour light:dark daily cycle at 26° C. and relative humidity of 65%.

C. capitata Germline Transformation

Plasmid microinjections were carried out into wild-type Benakeion strain embryos. The donor 795H1 and 795K1 piggyBac plasmids (500 ng/ml) were microinjected in combination with a helper plasmid encoding the ihyPBase transposase (300 ng/ml). The hatched G0 larvae were manually transferred onto larval diet and the surviving adults were crossed to virgin wild-type flies in a reciprocal manner. Fluorescence microscopy was used to identify marker-positive G1 progeny at the pupal stage of development, and selected adults were individually crossed with wild-type flies. Once established, homozygous transgenic lines were maintained through sibling crosses of 10 males and 20 females.

### Inverse PCR

Unique piggyBac construct integrations were analyzed through inverse PCR in selected marker positive G1 individuals. In brief, genomic DNA (gDNA) was first extracted via a modified protocol. The inverse PCR protocol was adapted from an established protocol utilizing Sau3AI (New England Biolabs®) and HinP1I (New England Biolabs®) restriction endonucleases for initial gDNA digestion, piggyBac-specific primers were used for sequential PCR amplification. Sanger sequencing was carried out using Genewiz Inc. services and the resulting sequences were analyzed using the latest C. capitata genome reassembly (GenBank GCA_905071925.1).

### DsRed Splicing Confirmation

Adult males and females from transgenic lines were separately collected in TRIzol reagent (Ambion). RNA was extracted and Maxima H Minus First Strand cDNA Synthesis Kit with dsDNase (ThermoFisher) was used for cDNA synthesis. In parallel. gDNA was extracted as described above. PCR was performed using Phusion High-Fidelity PCR Master Mix with HF Buffer (New England Biolabs®) or RedTaq DNA Polymerase 2× Master Mix (VWR Life Science) using primers designed in Geneious Prime 2023.1.2 (Table 8). The amplicons were visualized using a 1% agarose gel.

### Sex Sorting Assay

For all homozygous strains two consecutive generations, G9 and G10, were screened to confirm system efficiency. Parental crosses between 10 male and 20 female homozygous individuals were established and eggs were collected twice 3 days apart for each cross. The offspring were reared until adulthood under regular conditions. At adulthood, all flies were assessed by 3 phenotypic parameters. These included phenotypic sex characteristics (male or female), as well as fluorescence marker phenotypes determined via separate screening with the GFP (GFP+ or GFP−) and RFP (DsRed+ or DsRed−) filters. MVX-ZB10 Macro Zoom Fluorescence Microscope System (Olympus) was used for all imaging and fluorescence screening.

### Fitness Assays

To assess fitness costs of two copies of sex-sorting genetic cassette, the number of eggs laid and rate of egg hatching of homozygous cassette-carrying and wild-type flies were evaluated. Genetic crosses between 15 males and 25 females from each strain (H-001, H-002, K-001, K-002) were set up in biological triplicates. Simultaneously, crosses of 15 male and 25 female wild-type Benakeion strain adults were also established in triplicates. After 5 days, eggs oviposited within a 5-hour window were placed onto black filter paper on top of the larval diet and counted using ImageJ. The hatching rate was established 4 days after initial egg collection similarly through counting the remaining unhatched eggs.

### Example 9—Establishment and Characterization of Fluorescent SEPARATOR Strains

To engineer the SEPARATOR genetic cassettes for Ceratitis capitata, the transformer (tra) intron of C. capitata or A. ludens was utilized to generate 795H1 or 795K1 construct, respectively. Importantly, both constructs included the DsRed coding sequence separated by the tra intron and expressed under the Opie2 promoter, and a dominant Hr5-IE1-eGFP marker (FIG. 23A). Germline transformation was used to induce piggyBac-reliant integrations for 795H1 and 795K1 constructs (Table 9). Two separate strains for each construct were successfully characterized through inverse PCR (H-001 and H-002 for 795H1; K-001 and K-002 for 795K1) (FIG. 25 and Table 10). Homozygosity for all strains was achieved via sibling crosses at G2 thereafter. No DsRed+/GFP+ males or DsRed−/GFP+ females for any of the 4 homozygous strains were noted during routine screenings for 8 consecutive generations (from G3 to G10). This was indicative of sex-specific splicing of DsRed, and thus the success of tra intron selection for both C. capitata and A. ludens. For verification, the strains were expanded and the entire populations characterized at G9 and G10 (FIG. 24A). As expected, no DsRed+/GFP+ males or DsRed−/GFP+ females were detected. Transgenic individuals with only two phenotypes: DsRed+/GFP+ females (48.8%) and DsRed−/GFP+ males (51.2%) were noted across the four strains (Table 11). Chi-squared tests for observed and expected sex ratios showed no statistical significance for any of the four strains. Overall, 100% transgenic flies expressed the desired fluorescence phenotypes across both C. capitata (795H1) and A. ludens (795K1) tra intron-harboring medflies across G9 and G10 with a total of 3,787 flies counted.

TABLE 9
Injection summary for 795H1 and 795K1 constructs

| | | | | G1 | G1 |
|---|---|---|---|---|---|
| Transformation | Eggs | G0 Hatched | G0 | DsRed+/GFP+ | DsRed−/GFP+ |
| Construct | Injected | Larvae | Adults | ♀ | ♂ |
| 795H1 | 250 | 210 | 84 | 31 | 11 |
| 795K1 | 250 | 141 | 52 | 4 | 2 |

TABLE 10
Genomic integrations determined through inverse PCR

| Transformation | | | | |
|---|---|---|---|---|
| Construct | Subline | Scaffold | Annotation | Integration Site |
| 795H1 | H-001 | 2 | 77 806 718 | CACATCTCTATTAA-PB-TTAACAGCTATAAT |
| | H-002 | 1 | 99 273 433 | GTAAAACTTTTTAA-PB-TTAAAACCAATTCA |
| 795K1 | K~001 | 1 | 87 907 601 | CCAGCAGTGATTAA-PB-TTAACGTAGGTACG |
| | K~002 | 2 | 12 057 725 | TTGGCAACTGTTAA-PB-TTAAACAGACATCA |

TABLE 11
All adult flies in the G9 and G10 generations were phenotyped
by sex and screened for GFP and DsRed fluorescence markers

| Transformation | | DsRed+/GFP+ | DsRed−/GFP+ |
|---|---|---|---|
| Construct | Subline | ♀ | ♂ |
| 795H1 | H-001 | 167 | 221 |
| | | 249 | 231 |
| | H-002 | 156 | 219 |
| | | 216 | 229 |
| 795K1 | K-001 | 106 | 105 |
| | | 243 | 227 |
| | K-002 | 330 | 307 |
| | | 380 | 401 |

### Example 10—Fitness of Fluorescent SEPARATOR Strains

To assess whether the SEPARATOR cassettes are associated with notable fitness costs, the rates of egg laying and egg hatching rate were assessed for the four homozygous strains (Table 12). Sibling crosses of homozygous individuals were conducted for each strain alongside wild-type controls. The egg laying rates were variable across wild-type and transgenic strains, although no statistically significant differences between wild-type and transgenic lines were determined through Kruskal-Wallis test and Dunn's multiple comparison test (FIG. 24B). It is of note, however, that there was a notable reduction in H-002 strain egg production. Meanwhile, the egg hatching rate of the 795H1-harboring strains with the endogenous tra intron was similar to wild-type (FIG. 24C). The exogenous intron-containing 795K1 strains had reduced egg hatching rate compared to non-transgenic flies. Specifically, the egg hatching rate of K-001 was significantly reduced (p=0.019), while the egg hatching rate of K-002 was reduced insignificantly (p=0.072).

TABLE 12
Egg laying and egg hatching data

| Construct | Strain | Replicate | Embryos | Hatched larvae |
|---|---|---|---|---|
| N/A | WT | 1 | 171 | 133 |
| | | 2 | 164 | 134 |
| | | 3 | 210 | 173 |
| 795H1 | H-001 | 1 | 249 | 212 |
| | | 2 | 88 | 73 |
| | | 3 | 211 | 161 |
| | H-002 | 1 | 50 | 42 |
| | | 2 | 132 | 98 |
| | | 3 | 110 | 93 |
| 795K1 | K-001 | 1 | 104 | 44 |
| | | 2 | 89 | 52 |
| | | 3 | 225 | 90 |
| | K-002 | 1 | 146 | 81 |
| | | 2 | 253 | 179 |
| | | 3 | 246 | 161 |

### Example 11—Fluorescence Patterns of SEPARATOR Strains

It was observed that the GFP fluorescence signal across the 4 homozygous strains was visibly similar. DsRed signal, however, was visually more intense in the females harboring the endogenous C. capitata tra intron (H-001 and H-002), compared to the females with the A. ludens tra intron (K-001 and K-002), irrespective of GFP fluorescence intensity in homozygous individuals (FIG. 26). The patterns of fluorescence marker expression were further investigated throughout the C. capitata life cycle (FIG. 23B). A clear distinction between males and females was observed immediately upon hatching as larvae and increasingly throughout all later stages of the life cycle. Specifically, the signal was easily recognizable throughout the first, second and third instar larvae; pupae and adult development stages. At the late egg stage, GFP fluorescence is very clear in all individuals, whilst DsRed fluorescence was more difficult to differentiate (FIG. 27). This is because all eggs had a DsRed signal of variable intensity, which was stronger than in their wild-type counterparts.

Additionally, reverse-transcription PCR (RT-PCR) was carried out to amplify Opie2-DsRed for molecular verification of the sex-specific nature of DsRed splicing. cDNA, synthesized from total RNA, and gDNA were used to validate this phenomenon from adult males and females harboring either 795H1 or 795K1 (FIGS. 28A-28D). gDNA-derived fragments of similar sizes were observed in both sexes, and cDNA-derived fragments of different sizes in males and females. The female-specific cDNA-derived fragments for both C. capitata (795H1) and A. ludens (795K1) tra introns expectedly amplified as single shortest fragments. One of the two male-specific transcripts was detected for 795H1 males, while multiple bands were observed for 795K1 males. These were more challenging to distinguish for the 795K1-harboring males as there are 5 alternative male-specific transcripts of tra in Anastrepha due to the presence of 3 male-specific exons. It was deduced that DsRed in both cassettes is spliced in a similar fashion to the tra gene whereby DsRed translation is interrupted with male-specific stop codons. It was also concluded that the A. ludens tra intron, despite incomplete sequence similarity, is functionally suitable to replace its endogenous counterpart in C. capitata.

1.-34. (canceled)

35. A method of sex-sorting a plurality of insects based on sex-specific gene expression, the method comprising:
(a) delivering an exogenous nucleic acid molecule into an insect from a plurality of insects, wherein the exogenous nucleic acid molecule comprises a promoter region, a male-specific splicing module, a fluorescent marker gene, and a transcription terminator;
(b) detecting male-specific gene expression of the fluorescent marker gene in the insect; and
(c) sorting the insect from the plurality of insects based on the detecting of the male-specific gene expression in step (b),
thereby sex-sorting the insect based on the male-specific gene expression.

36. The method of claim 35, wherein the exogenous nucleic acid molecule further comprises a piggyBac inverted terminal repeat located at each end of an effector region.

37. The method of claim 35, wherein the promoter region comprises a constitutive, inducible, or tissue-specific promoter.

38. The method of claim 35, wherein the promoter region comprises a CMV, CBA, CAG, Cbh, EF-1α, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, U6 promoter, a Hr5IE1 promoter, or an OpIE-1 promoter.

39. The method of claim 35, wherein step (a) comprises integrating the exogenous nucleic acid molecule into the genome of the insect.

40. The method of claim 35, wherein step (c) comprises sex-sorting the insect at an early larval stage, wherein the early larval stage is an L1 larvae stage.

41. The method of claim 35, wherein the male-specific splicing module comprises an endogenous or modified sex-specific exonic sequence and a modified male-specific intronic sequence.

42. The method of claim 35, wherein the insect comprises a mosquito or a fruit fly.

43. The method of claim 42, wherein the mosquito comprises a mosquito species from Aedes, Anopheles, or Culex genera.

44. The method of claim 42, wherein the mosquito comprises a Anophneles gambiae, Anopheles stephensi, Aedes aegypti, or Aedes albopictus.

45. The method of claim 43, wherein the male-specific splicing module is derived from a doublesex (Dsx) gene in the mosquito species from Aedes, Anopheles, or Culex genera.

46. The method of claim 35, wherein the fluorescent marker gene comprises a DsRed gene, an eGFP gene, or any combinations thereof, and wherein the transcription terminator comprises a SV40 poly(A) signal.

47. A method of identifying the sex of an insect based on male-specific gene expression, the method comprising:
(a) delivering an exogenous nucleic acid molecule into an insect, wherein the exogenous nucleic acid molecule comprises a promoter region, a male-specific splicing module, a fluorescent marker gene, and a transcription terminator; and
(b) identifying male-specific gene expression of the reporter gene, thereby identifying the sex of the insect based on the male-specific gene expression.

48. The method of claim 47, wherein the exogenous nucleic acid molecule further comprises a piggyBac inverted terminal repeat located at each end of an effector region.

49. The method of claim 47, wherein the promoter region comprises a constitutive, inducible, or tissue-specific promoter.

50. The method of claim 47, wherein the promoter region comprises a CMV, CBA, CAG, Cbh, EF-1α, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, U6 promoter, a Hr5IE1 promoter, or an OpIE-1 promoter.

51. The method of claim 47, wherein step (a) comprises integrating the exogenous nucleic acid molecule into the genome of the insect.

52. The method of claim 47, wherein step (b) comprises sex-sorting the insect at an early larval stage, wherein the early larval stage is an L1 larvae stage.

53. The method of claim 47, wherein the male-specific splicing module comprises an endogenous or modified sex-specific exonic sequence and a modified male-specific intronic sequence.

54. The method of claim 47, wherein the insect comprises a mosquito or a fruit fly.

55. The method of claim 54, wherein the mosquito comprises a mosquito species from Aedes, Anopheles, or Culex genera.

56. The method of claim 55, wherein the male-specific splicing module is derived from a doublesex (Dsx) gene in the mosquito species from Aedes, Anopheles, or Culex genera.

57. The method of claim 47, wherein the fluorescent marker gene comprises a DsRed gene, an eGFP gene, or any combinations thereof, and wherein the transcription terminator comprises a SV40 poly(A) signal.


SEQUENCE LISTING (58 sequences)

SEQ ID NO: 1 - DNA, 11920 bp, synthetic construct
  source 1..11920: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatctgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacgggcccgcgtgcaaaagcgtgtttttttgcagtgcaaaaaagttggtggtggggaggccaccgagtatgccgcggaattgatcggctaaatggtatggcaagaaaaggtatgcaatataataatcttttattgggtatgcaacgaaaatttgtttcgtcaacgtatgcaatattttttattaaaagagggtatgcaatgtattttattaaaaacgggtatgcaatataataatcttttattgggtatgcaacgaaaatttgtttcgtcaaagtatgcaatattttttattaaaagagggtatgcaatgtattttattaaaaacgggtatgcaataaaaaattatttggtttctctaaaaagtatgcagcacttattttttgataaggtatgcaacaaaattttactttgccgaaaatatgcaatgtttttgcgaataaattcaacgcacacttattacgtgaggtaccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgacttaattaacaaaatgaacgacgaacttgtcaaacgatctcaatggctcctggagaagctgcgatacccctgggagatgatgcccctgatgtacgtgatactgaaaggcgccgacggagacgtcaataaagcgcgccaacggattgacgaaggtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattctcatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaattagttaaagtttcgagagcattgtcaatatcaagtgttgttagcaaacaaatgctaacatcaagattactatcgatgtttgattcacatgtattccaatcagctcgtaaaacgtcaacatcccaccgcaaaatcgctagtgtttggaggggattttaacctccaaattgccaaataacctccaaatcatcacctccaagttagttctaatacactccgttatatgaaatatggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcaggtcaagccgtggtcaatgaatactcacgattgcacaatctgaacatgttcgacggtgtagagttgcgcagtacgacgcgccagtccggatgatagactttttacacgatcagcacgacccactgcgctgcggcaaaggtcgaaccgaaacaagaataaaccacgaagatcagatcgattcgacggaagaagcaatcgaatgcaaagaagaatcggaacgaagaaaactctaaagcatcgcatatttacaaagcataacggaaaacccgcaagttcaaactagtgattagtgtaagatgaagcaaagcagaaatgtagtatctagatttttcaacgttagtttacaaagataaaaaatgaggttggacatacaatcgtgggtattcgtctgagttcgtcacaactgcaccggaaactgtgaaacagaatagagccaacctgtgcgcggagaatgttgaggtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacagtgcaaatgctgtttaacgataatagcgacatgcagccattctggggctaccacgtggagctctacttgggagacagcgttcctaaagagtgtgaaagtgcaaacaagggaggaaaccaagagtgcaaagcaagtttagagggaaaatttaaaaaatgcaaaacagcagtagtacttaactttgaagattgtgtttcgaaagccgaagtgtgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatcggagaaacacttatctctctggagcttgcatgccattgcgagcgtattttggtagctggccgttgccaaacggctccgcaggtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcagcgtgcagtgttatgaagatatccacagtataaaatattattttattttaaattctatgctgattatcaatgtgttactagtggcttttcatactcatgttgcgagctcgatttggcgcacggggtcatctacacctgatacctttagggtcgttgggggaccacttagcgtgcacgtacggacattcaaaatgttgttcaaatttttttcttaccaagacgagcactttacaatgacaaacttataatatttcgcatttttgcgataaagtcaatggcattatgaggcagagggaagcatcatcttccttatgctatccaagtaatttgccattgctttcaagaaatcgaggatttcatcatgccaaatcagttatcttgtatcaaggcatgtatgtatgttgtttgaagcaactgtataactgtttgaaactatctaattggtgagctcgtttcatttagtatataataatgataattgctatggagacgttatttactagcaagtgatttgacgacctgaaatcggaacaaatagacaacgtttttataaatacaataaatcagaactttccattattgggtacaaagagttgcgctatttcgatactgtcagatcagattttccagcacaacgataccttgatatgcgataacttagaattagaccttcaaatccatctctccagctatgaacagtcatatagataaagccaatggcgttatgaggtagcggaaagcgtcatctttccaatgctatctaagtacataatttgctatagctttctattaatcgtagtttgagagatgcaaagtcagttatctcgtatcaaggtttgattgttttggaaattagctaaacagttgacattatcacccgtctttaggggataagcgcatacaaatgtgtatttagttgttcattgaagtaacgtaagataggcaagtatggaaacgagctcaccaaacgtcgaaatacgtctaataaatttgtgttcagcaggatggttcaaaatttatttgcatcacctcaaaattacagtacctagtgctgtttgtgacaaacatcaaaaggtaaaatcaaactcgtggcgtcgtgcaatctccatagaatgaacaatttctaaccgtatttgatggaaagacattgagtatactaacctcttaacagcattacacttttctataaacaataaataatttgttctattttacattttctttccccactttcgccccccaataattcaatccctcaaacaggatacgacatttgttgcatctactttccgaagcgttccagcagacacagacactggccggacgaggagaacatctccgtcacccgcactccgtctgcgtcacggtcgccatgtgccgattttcgtacccggtcacagtccagctcgccggatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttgcggccgcaattgatcggctaaatggtatggcaagaaaaggtatgcaatataataatcttttattgggtatgcaacgaaaatttgtttcgtcaacgtatgcaatattttttattaaaagagggtatgcaatgtattttattaaaaacgggtatgcaatataataatcttttattgggtatgcaacgaaaatttgtttcgtcaaagtatgcaatattttttattaaaagagggtatgcaatgtattttattaaaaacgggtatgcaataaaaaattatttggtttctctaaaaagtatgcagcacttattttttgataaggtatgcaacaaaattttactttgccgaaaatatgcaatgtttttgcgaataaattcaacgcacacttattacgtgacctgcaggcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 2 - DNA, 11920 bp, synthetic construct; identical to SEQ ID NO: 1
  source 1..11920: mol_type = other DNA

SEQ ID NO: 3 - DNA, 60 bp, synthetic construct
  source 1..60: mol_type = other DNA
atcgtgaacaaccaagtgacttaattaacaaaatgaacgacgaacttgtcaaacgatctc

SEQ ID NO: 4 - DNA, 50 bp, synthetic construct
  source 1..50: mol_type = other DNA
gcgattttgcggtgggatgttgacgttttacgagctgattggaatacatg

SEQ ID NO: 5 - DNA, 50 bp, synthetic construct
  source 1..50: mol_type = other DNA
catgtattccaatcagctcgtaaaacgtcaacatcccaccgcaaaatcgc

SEQ ID NO: 6 - DNA, 50 bp, synthetic construct
  source 1..50: mol_type = other DNA
ctcataatgccattgactttatcgcaaaaatgcgaaatattataagtttg

SEQ ID NO: 7 - DNA, 50 bp, synthetic construct
  source 1..50: mol_type = other DNA
caaacttataatatttcgcatttttgcgataaagtcaatggcattatgag

SEQ ID NO: 8 - DNA, 55 bp, synthetic construct
  source 1..55: mol_type = other DNA
gttatcctcctcgcccttgctcaccattccggcgagctggactgtgaccgggtac

SEQ ID NO: 9 - DNA, 902 bp, synthetic construct
  source 1..902: mol_type = other DNA
tgcaaatgctgtttaacgataatagcgacatgcagccattctggggctaccacgtggagctctacttgggagacagcgttcctaaagagtgtgaaagtgcaaacaagggaggaaaccaagagtgcaaagcaagtttagagggaaaatttaaaaaatgcaaaacagcagtagtacttaactttgaagattgtgtttcgaaagccgaagtgtgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatcggagaaacacttatctctctggagcttgcatgccattgcgagcgtattttggtagctggccgttgccaaacggctccgcaggtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcag

SEQ ID NO: 10 - DNA, 39 bp, synthetic construct
  source 1..39: mol_type = other DNA
gttgccaaacggctccgcaggtactgctattggaggttg

SEQ ID NO: 11 - DNA, 41 bp, synthetic construct
  source 1..41: mol_type = other DNA
gttcttggaggagcgcaccatccggcgagctggactgtgac

SEQ ID NO: 12 - DNA, 21 bp, synthetic construct
  source 1..21: mol_type = other DNA
atggtgcgctcctccaagaac

SEQ ID NO: 13 - DNA, 56 bp, synthetic construct
  source 1..56: mol_type = other DNA
gaacagctcctcgcccttgctcaccattactacaggaacaggtggtggcggccctc

SEQ ID NO: 14 - DNA, 30 bp, synthetic construct
  source 1..30: mol_type = other DNA
cggctaggttaaacgagtagtgtccacctc

SEQ ID NO: 15 - DNA, 34 bp, synthetic construct
  source 1..34: mol_type = other DNA
gtaatttactaacgtagaaatcctgtgctggcac

SEQ ID NO: 16 - DNA, 24 bp, synthetic construct
  source 1..24: mol_type = other DNA
aaaaataaggtcagcagccatgac

SEQ ID NO: 17 - DNA, 24 bp, synthetic construct
  source 1..24: mol_type = other DNA
gtcatggctgctgaccttattttt

SEQ ID NO: 18 - DNA, 31 bp, synthetic construct
  source 1..31: mol_type = other DNA
agacattccaaattcaagttaacaaattaat

SEQ ID NO: 19 - DNA, 31 bp, synthetic construct
  source 1..31: mol_type = other DNA
attaatttgttaacttgaatttggaatgtct

SEQ ID NO: 20 - DNA, 21 bp, synthetic construct
  source 1..21: mol_type = other DNA
cttgctctggagtccgttaaa

SEQ ID NO: 21 - DNA, 22 bp, synthetic construct
  source 1..22: mol_type = other DNA
cctgagcagataaccctctttc

SEQ ID NO: 22 - DNA, 21 bp, synthetic construct
  source 1..21: mol_type = other DNA
ggtcgtttcggtaacgtagaa

SEQ ID NO: 23 - DNA, 22 bp, synthetic construct
  source 1..22: mol_type = other DNA
gtcatcagttgtcggcatttac

SEQ ID NO: 24 - DNA, 55 bp, synthetic construct
  source 1..55: mol_type = other DNA
ccgcaacctgtctctggtgggatccatggtgcgctcctccaagaacgtcatcaag

SEQ ID NO: 25 - DNA, 64 bp, synthetic construct
  source 1..64: mol_type = other DNA
acgccatccaaccgccgccgcaacctgtctctggtgatggtaattttaaaagcatatttttttc

SEQ ID NO: 26 - DNA, 64 bp, synthetic construct; identical to SEQ ID NO: 25
  source 1..64: mol_type = other DNA

SEQ ID NO: 27 - DNA, 57 bp, synthetic construct
  source 1..57: mol_type = other DNA
aattcaacgcacacttattacgtgaggtaccgcgcccatactcggtggcctccccac

SEQ ID NO: 28 - DNA, 60 bp, synthetic construct
  source 1..60: mol_type = other DNA
ggccgactgttttcgtatccgctcaccaaacgcgtttttgcattaacattgtatgtcggc

SEQ ID NO: 29 - DNA, 60 bp, synthetic construct
  source 1..60: mol_type = other DNA
tcaaagaaaaaaatatgcttttaaaattaccattttgtattgtcacttggttgttcacga

SEQ ID NO: 30 - DNA, 58 bp, synthetic construct
  source 1..58: mol_type = other DNA
tcgtgaacaaccaagtgacaatacaaaatggtaattttaaaagcatatttttttcttt

SEQ ID NO: 31 - DNA, 60 bp, synthetic construct
  source 1..60: mol_type = other DNA
accccggtgaacagctcctcgcccttgctctatagataccatagatgtatggattagtat

SEQ ID NO: 32 - DNA, 60 bp, synthetic construct
  source 1..60: mol_type = other DNA
atactaatccatacatctatggtatctatagagcaagggcgaggagctgttcaccggggt

SEQ ID NO: 33 - DNA, 60 bp, synthetic construct
  source 1..60: mol_type = other DNA
atcagatcggttatactatatagtgggtaccattttgtattgtcacttggttgttcacga

SEQ ID NO: 34 - DNA, 60 bp, synthetic construct
  source 1..60: mol_type = other DNA
tcgtgaacaaccaagtgacaatacaaaatggtacccactatatagtataaccgatctgat

SEQ ID NO: 35 - DNA, 60 bp, synthetic construct
  source 1..60: mol_type = other DNA
caccccggtgaacagctcctcgcccttgctctatgtgaaaagagtgtgcggttagtcaat

SEQ ID NO: 36 - DNA, 60 bp, synthetic construct
  source 1..60: mol_type = other DNA
attgactaaccgcacactcttttcacatagagcaagggcgaggagctgttcaccggggtg

SEQ ID NO: 37 - DNA, 60 bp, synthetic construct
  source 1..60: mol_type = other DNA
tctgatccgatcgaatatgtgtatatataccattttgtattgtcacttggttgttcacga

SEQ ID NO: 38 - DNA, 60 bp, synthetic construct
  source 1..60: mol_type = other DNA
tcgtgaacaaccaagtgacaatacaaaatggtatatatacacatattcgatcggatcaga

SEQ ID NO: 39 - DNA, 61 bp, synthetic construct
  source 1..61: mol_type = other DNA
ccccggtgaacagctcctcgcccttgctctacgtggaagtggaagaagaggtgttaatcac

SEQ ID NO: 40 - DNA, 61 bp, synthetic construct
  source 1..61: mol_type = other DNA
gtgattaacacctcttcttccacttccacgtagagcaagggcgaggagctgttcaccgggg

SEQ ID NO: 41 - DNA, 60 bp, synthetic construct
  source 1..60: mol_type = other DNA
ttcataaaataaaatgtaggttacaattaccattttgtattgtcacttggttgttcacga

SEQ ID NO: 42 - DNA, 60 bp, synthetic construct
  source 1..60: mol_type = other DNA
tcgtgaacaaccaagtgacaatacaaaatggtaattgtaacctacattttattttatgaa

SEQ ID NO: 43 - DNA, 60 bp, synthetic construct
  source 1..60: mol_type = other DNA
caccccggtgaacagctcctcgcccttgctctgtgggcacgatgattttttatattagta

SEQ ID NO: 44 - DNA, 60 bp, synthetic construct
  source 1..60: mol_type = other DNA
tactaatataaaaaatcatcgtgcccacagagcaagggcgaggagctgttcaccggggtg

SEQ ID NO: 45 - DNA, 41 bp, synthetic construct
  source 1..41: mol_type = other DNA
caattgtggcgtttacagcatttgttatacacacagaactc

SEQ ID NO: 46 - DNA, 30 bp, synthetic construct
  source 1..30: mol_type = other DNA
gaacagcatctgttacagcgacacaacatg

SEQ ID NO: 47 - DNA, 29 bp, synthetic construct
  source 1..29: mol_type = other DNA
cgattcatcctaggctacaggaacaggtg

SEQ ID NO: 48 - DNA, 25 bp, synthetic construct
  source 1..25: mol_type = other DNA
catccaaccgccgccgcaacctgtc

SEQ ID NO: 49 - DNA, 24 bp, synthetic construct
  source 1..24: mol_type = other DNA
aaaaataaggtcagcagccatgac

SEQ ID NO: 50 - DNA, 24 bp, synthetic construct
  source 1..24: mol_type = other DNA
gtcatggctgctgaccttattttt

SEQ ID NO: 51 - DNA, 31 bp, synthetic construct; identical to SEQ ID NO: 18
  source 1..31: mol_type = other DNA

SEQ ID NO: 52 - DNA, 31 bp, synthetic construct; identical to SEQ ID NO: 19
  source 1..31: mol_type = other DNA

SEQ ID NO: 53 - DNA, 21 bp, synthetic construct
  source 1..21: mol_type = other DNA
cttgctctggagtccgttaaa

SEQ ID NO: 54 - DNA, 22 bp, synthetic construct
  source 1..22: mol_type = other DNA
cctgagcagataaccctctttc

SEQ ID NO: 55 - DNA, 21 bp, synthetic construct
  source 1..21: mol_type = other DNA
ggtcgtttcggtaacgtagaa

SEQ ID NO: 56 - DNA, 22 bp, synthetic construct
  source 1..22: mol_type = other DNA
gtcatcagttgtcggcatttac

SEQ ID NO: 57 - DNA, 19 bp, synthetic construct
  source 1..19: mol_type = other DNA
ccgcaacctgtctctggtg

SEQ ID NO: 58 - DNA, 22 bp, synthetic construct
  source 1..22: mol_type = other DNA
cgatggtgtagtcctcgttgtg